SEQUENCE LISTING (7 sequences)

SEQ ID NO: 1 - DNA, 41802 bp, Unknown
  uncultured marine microorganism HF10_19P19
ttgattgacctgcgtggtctgctgggtcatttcatttgttactgatttgctgggagacgggctgaaattcagcgaaagaacggttgtgcctttcggttgttcaacgattggtacaactttctgcacaccaaacgcattacgcagagaacctgaaatctgtttaaatttgggaacgttaaattcagcaaaagaaagaatcagcacaaagaaggccatgagcaatgtggccatatcagcgaatgttgccatccatgccggggcgccgacaggagggcatttcggacattcttcctgttcttcttcaatttcctgtgctgcatcttctgccattatatctgcctttatcgtgaaactaatgtttcagcttaagggtgttgctctggccttatgccatttccattttttcctgcatctttggtgcaagattagcaaccatctgatcttggatatttcgtggagattcactgcgggcgatgttacgcaggccggtgacgaccatgtctcgatacacaagttcatattcggtgtatttttccagtttgttcagcaaaggcaaaaacagaacattggcgatgatcgcaccatacattgtggtgagcaaggctacggccatcgcaggaccaatggcttttgggtcagacatattgcctagcatcaacacaagaccaatcagcgtgccgaccatgcccattgcgggggcaatatcaatccaggccttgaccgcattttggttgacttcgtggcggtttttcatcgccttaatctctttatttagctgggtcacaagtttagcctcatctgcgccatcaactagcatttgcagacctttctggaaaaacttgtcaggcacttcctgaccttcaagagccatcataccatctttacgggcaatccctgatagctcaaccatacgctctataagctcatccatctttttaattggtggtaaaaaagcttttgccatagctccaaagtgaccgagaaaaacaggcattggaacagcatagagaaccagaaacgcggttcccccaaaaacgatcaggatagatggcacgtcaacaaacggggcaattccgccacctgatatcatcgcacctaggatcatgccaatcccgccaattagcccaataagagacgctatatccataagcaattcacctgttttgcctgttatgtcatcagtaggcagttgaactgccgacgccctaaaaatgcaagtttgataccaaaaaaggtttaacactgtatttttggcctgttctgcctgcccggcaggatagacctctagaaaacatgtcctcaaacccactgctgtttttcacatgcttattatatataaatatgcttatatttgaaaatatgaagagagactcctatcaattatgtcagatttgcagttttctgcagcgaagagcatggctgatatgctttagtattggtctttttggcaacattggcgcgattttggctgtggctgcgcctgttcatgctcagataccaacaaacggggcttacgtaattaaggatgacaaggttattgatctgcgttctggtgtagaatggctgcgttgttctttaggtcagcaatttgatgagggggattgcacaggagaagtgctccgcctgacacagaatgaggtggctgaagctattctgattgccaaccgcgaactaggtgggatatggcgcttgcctacccgcaaagaacttgaatttctggtctgcaaaagctgtcctgcaccgaaaattgataaattcgtatttccaggaacagtttctgaaccctattggacagggcaacggaactggatttctccaaaaaatctctggtcagtcaattttatgaccggccacagctatggccggtttttcccctatcagcggctagcagtccgtctcgttcgcacacgctagtgctctttagtgtggtgacttgtcagtctttgtctttttttatgtgaccagaattttaatccaaataagaaaacaggcaggccagcaaaggcccagccaaacatctcgggtttcagtccagctagcttatctagtaagtcatttgccagcgcgccaaacacaatcacacctaaaatcatcgccagataaacccttaggatctggcaacgtcggcagggctcacgtttaacctctgtgcgggttgaaaacattttctgactcagctccctggttttaccgcatgcactacctgtaaatcatgcgaaacagcttggttttcttgtctgaattctgacatgaaggaaaatttttttttaatttaaatttttaacctattgaaaaaaataaaaaaattgtgttgaaaatctgacaaatccttctgtcaaacgtttattttgcaaaacttgtgccgtctgagcaccgtgattgtcgaagagtacatgaagggagacatgttatgcaattatcacagtccataaaactgcgccagaagcagtcgctggttatgacaccacagctgcaacaggcgattaagctattacagatgaccaatctggaattgtcccagcatttgcaggaacaaatgtatgacaacccatttctggaagtcaacggggcaactgaaacagagctgagcgaaacagccaaacatactagtggcccggcagactccgaagtactgaataagaacttgacagccaccgaaggtcaaaatgatgtatctgctttgcgcgaaagtgcagcactggctgatgatccaactcaaaaccaagaaattgagaaccgctttgacaccgctcttgttgatatgccgagtaatccaggtgttagaaataaatttgctgatggtgattttgacccgattgacagcctaacttctgaagacgggttttacccgatgctatttaaacagatgaatttgactttttctgacccagctgaccggattattgccacacactttatcaatgcacttgaaccatctggctggattggcaattcggtagaggctatcgctgaagaaagtggctgctcagtagagcaggctgagacagtactagcagcgttacaagggtttgagcctgctggtctgttcgcccgcgatttggctgagtgtttaaaaatccagctaattgagaacaatcagttttcccacttgtttggtcagcttctggataatctagaacaacttggccgcggagaaattaaacaactggctcgcaaatttaagtgcgcgcccgaagacattgttgagatgctctcgattatccgcacgttaaaccctaaaccaggtgaacatctagggattgaattccgagaaatgcccagcccggacgtcattgtgacacgcaagaaacagggctggtcagttgaactaaatcggtcaaccctgccatcggttttggtgaatgaacattatgccgagctgatggctaaacagaaacgtactgaccaggccgtgcaggattattccgcagaaacattaacaaatgcgcgctggctaaaacgcgctgttgagcaacgtaatcagacaacattgaagatcagtgccgagataatccgtcaccagactgactttcttgaaaaaggtctagactatctcaaacccttgtctttgcgggatgtggcgctggctgttggtatgcatgaaagtacagttagccgagtgactactggtttgctaatttccacaccgcgaggcggcatgccacttaagtccttcttcagcgtcaatatcgcttcgcgtgattcagatatgaatgcctctgctgcttccgtgcggaatatggttaagaaaattatctcacaagaagttccagggaaaccgcttagcgatgaagccatttcaaaagtcatttcagaacagggcattgacttggcccgccgtacagttgcgaaataccgcgaaatgctgaacattccatcctcttctcagcggcggatgcgagccagattatctgaattccgctaagaaacggataaaaacttttcagtttctcccaaagcctcagttgaatgcggctttactttccccggcggttcgaaaccgtcggggttttttaactaaaaaagagttgcgggagtagctcagcgtttagcaaccattgcagagagttgtatggtcatttcacgttcttgctcgctggcttcacattcatgcaaatacagtcccagcggtatcagattctcacgaataggttcatcattctgatcaaattcaacgaatttgatggccattttttccgcaggttgtttttttgcacgcatatttctagtccctcttatccgatgcctaacaatgggcaagcagagtaaaagctgcaacctctgtgccataccgggaacaaagttgctcaacaggttaaaaaagcagaaaattggttgcttacatctcgcctgacaaagcaccagtgattgtcaatttatggccataggtgaaaaaatcttcagggtcctttgatttgccatcaatccggatctcataatgaagatgcatgccatctgaatggcctgtattacccattgtgcccaatatatcctgtgtagttacaaagctgcctttgctgaccagcacagatgatagatgcgcataaacagtctcaatgccatgggcgtgctggaccctgaccactttaccaaaaccgccatcccgtccagaaaatgttacaatgccactggctggaggacgcagcttggctttccaggttcctgccaggtcgactccgtgatgaaaccgcgtcactccggtctttttattggtgcgcatgccataagggcttgtcacataataatagcgcaaaggggcctgcaatggcagcatatttaacaattcacgccagctgtctcggtaaagatacagcgcaggcacatgttcttcagcagcggcaagtgcagggttccattgttcaggctcactgtcaggagttattcccagtttgtccagcatgattttaatacgctcaacctctttaagcactttggcaaaacgtctatgcagctttatttccggagtttgcagaggtgggttgtgcagtccgtcaaaactggcaaattcagggtccatgggctcaggcgcagggcgagcaacaggcgtagctaccacattaactttccgttcagcaagttctagattgctgtctttctgaagggccgatagtgaggagactaactcatcgtttggctcttcgatatctgaatattgctcagcctctataatgatctcctgctggagtgccaagaggctcgttttttgctcgttattctcttttaaaggtgatttttgttcggatacgttgtccaggcctggcaaatgggtttggccatcaaatgatggcaaatgtggatacaataatgtcgtggtaactgaatcctccaaaagaggtggccaggctgtcttagcatcttttgggtctgtttgttcaaacgtcatgggaatgcgtgtggccagctgttttatgaggctgtgggaggacaatgatgaaaaagagggtagatttggcaaggcaggggccaataaaataagaacagtaaggccaagccctccagtgactccagagataatatgagtgggccgtaggcagaattctattgggccgttagcgcttaaaaataagaatcgtgtttcgcggaggaacagacgattatgcgttgttgaaaagcgcatgtgttcactgtcggaatcaacagcgggcggcagaactgaagccagtttatgtatcacccagctgaacataaccagtccctatttgagcagtaaaatcgccatcaatatcacatttgagattagctgcagaattatcagcgtgattattaggccacttgactgccgattcttgatattaaggttaaaggaaggcattgacatggctttggtcagcgcgttatgcatggcaatggtagtatccgtcacgccggtccgatttttttccaaagattgttgatcatcaaatgacgcgtcgccttctggatgattaatcgaagcatcctgaattgagaaatttacatcatcgcgcaggttctcatcgggcagattgtagctgtgtttggacacaatcgcctctgacacctcatttctgattttttcaatccgtttgcggatatcaattacgtcaacgaccattaatctttgcctttgccttgctcaaatgcgccaacactctatactgttcaacgtctcaaacaagcaagcatttatagctgatgtagtgtttcgagttattttggtttattctacattagtaaatcccttcttgttgagtcattgtccctgggaacattagagtaaaccgtttaacctattgatacagcagtataaattatgtttggaaataaaccggcccaagggtcttcaacgccgcagacagcaaatataggcgcaagtatacgcattaagggtgagattgattcgcctgagcatgtctttttggcaggtagtcttgatggtgatattacctgtgatcagctaacagttgatggcacaggccggatcacaggcaagatatcagctaagcttgctgtaattgatggccatgttgatggtgatattgtggttgaccggttgcagattaaatcaaacgctgtagtgaagggttcactcacttacaagagcattgaggtggctgaaggcgcgcagattgacggcaatttcgtgcaagtcacgcaaaagccgcaaaaagacgcggtcactctaccaaaaactgaaaaataaacaggtagagtatgtcagaaaccgtgtcccgtaagtcagttattggcgcaggtgcaaccttcactgggaagctggttaaggcgtctgcgctggatattcacggtgaggttaatgccgatgtctcgacagatcgtcttcatcttcatgaaggggccagactagcgggagatcttgacattaagctgggagtgtttgccggcgaatataagggtaggatgagagcttcaagcgtttgggtaatgcgcacagctcgcatttttggcgagattgaatacaacgcgctgcaaatggacagaggtgcagcactgaactgtcacattttgcataattgggtagaacttaaagatgcaggtatagatcctttagaaataacagaagccgggtttttaacggacctacatgatcttggaccgtccgacaagacagactcacaggcctccgatgagtgattcaacttatattggtgttgatttaaagttgacaggtgtcttccatgcccctggacagactgttgtaattgccggtcgctttgaaggcgaaatgacagcaggctcagttgagatcaccaaggaagctgtatttgagggacttgtcagggcaactgaaatggacattgcgggccgcttcaatggggtgcttgaaacagaccatctgactgtttcggaacaggcagtagttgcaggtgaaatggtcgctcatgcgttatcagtggatgctggtgctgatatttctggcactgtcaggcgcaagcctgatcagacaagctcgtcatcgagctgagcaaggatttcatcacaaatttcagctgtttggatacagtcttcgaaaaaggccagtgtctgcgcgcgaccatggttttggaacaattgcattgccgcgtagtggcccgctgccatggccactgcgtcaggcatggcttgttcacgagctttatcctccagcatattgtcaatttccaaggtcagcttttcgaaagcctgcaccctaataaatttgtcagctgttcgtggcctagctttgcgattcacttttcgctctgctgcacgacgttccctgtaaaggtcaagttcaatgatattaaacatccgttcacgtttcctctcaagcaggcttctgagggcatctcagcatccgacctttcatgaaattcagccggctagaatcgtcaacacttacagtttggtcctgatagatatcaagcggaacaataacactggcggctagctgatgggcttgattgcgcataaggatcagggcttcatcatagctgccataatgatgcacctcaacaatgccgagggtccgacatccggccagatacgcatcagacacgtttgaaaaactgatcgtctcagaaccaggcagagcctgtggtttgttggcttcaagcttagcttgacgctgcttttctttgaactgatgcaattcagcccagtccgcagcacttaccactaacaaagattcattacgcccatctacaaaaaactggcaagatgacagccagattaaagctgaaaataaacttgcgagcatgcaaaatctgcgaagcatatcctctctcctgttttgaggtaatttgctggtatctgtgagatcacctatgtctgatcatcacagctttggttataatcttctcataaagtataaacggcgatcagtgatatttatttaatttagattaatgggataggcaaagatcatttttgtggtcacaggccctatacatcatctattttatggcgttacactcggttatgtttgcagcggcctcatcttcaaggtgtggcagtgctgaaaaaagaatgggttcagctgtgtcattgcaagtcaaaacgaatcgaccattttataaagcaattttatcaataggtatggctgttatcgtcgctgctatcctaatgatagggcctgctttcagcgaagggcgtatttatgaggggtctgaggagaaagcacattgctcactgtgggataatctgcgtctgaaatggccacaaacaatcttgggccgcgagaaggcgaaatgccgccgcaaggctaaaccgcccacacaagtcacaaatatatgtcgcctgcgccgccaatatattgatcctgaaacaggctcgcgcatgtgtgtttatatgcgtcagggccgcgaccttgaggacacatctgtcagcatgtcaccctctttgcaatgccagcgcacctatacttgccgaagtgatgactgacctggtgacaagttgccgtcatctggctcagctctacagataatttgccttatttcgcagcactacattgaatgaaattgacagacgcggcacatccactaagtttggatgcacaagatgatgtagaaaagcaggccagaggaagagttcaccagcgtctggtagccgtctgaattcagggtcaaactgaccatcaccgcgtacagctcccatgttggcctgtgggcgagggtcaaagaagctaatcgctcctgggttcaaatcagaccgaaaggtatttgcctcgccttgatcaggcacattcaaataataggttccggacagccaggcatgcgggtgattatgcagattgtgataatcgcctttgaaattcacattaggccaggcctgaagcgtccaatccagcggatagctaatgcctaattcctgagcatagtcatggaccgctttgtcaaaacactgacgcaaccaaagcacagcaggattttcatcctccaacaaattctggtccagataatttgttgtcatctgctcttgttcagcattcttttctagaaccagaccgctcagcatggcattggcctgttcatgcccaggcaaggtgacggccataaacaaagttggccataatcggtggaacgtggggctgcctggtgctgcctgttggggagtgtcagccatggtttcccttctttgaatgctagtaattgatgaaacaacaaattgatctattggtatcattaccagtatataaattgtatggcattatgttctgtcgctgattttcatgaaccggtagcatgatacttgctgacgttgagcatttaaggaggcaaaaatggcacttttcgatcaggtggataagggcggcttgcttgaatattcagttgtattcaatgaccgttcgctcaattccatgtcgaaatcttttcggggtgtaatgcaggatctgtctgtaggtctgaagtcagcttataacgcagaagcgtcgatactagttccaggcggtggtacgtttggtatggaagcgatcgcacgccaatttggcacagatgccaaatgtcttgtcatccgcaatggatggttctcgttccgctggtctgaaatttttgaaaaaggccggatcgcctctgataccaaggttttgtatgccaggcagcagcaggcagaggccggcgaaaatacgcaccagccctttgcgccttctcccttggcagatattcagcagcagattgctgactacgcccctgatgtggtctgtgctccgcacgtggaaacctcagcagggatggtcctgcctgatgactacattgctggcattgccgtggctgctcataaggctggggctatttttgtgctggactgtgtggcttctggcgcgctgtacgtggacatggcaggcctgggcgttgatgtgcttctgacggcgccgcagaaaggctggacagccagcccttgtgctggagtggttatgctgggtgcgggcggtcttgaccgtattgaaaagaccacaagcacttcatatgctgcagatttgaaaaaatggctgtcgatcatgcaggcttatgaaaatggggggcatgcatatcatgctaccatgccaaccgacgggctgcgtctgtttcgtgatgcggttcatgaaacaaaggatatcggctttgccttggtcaaacaggcgcagattgatttgggggtcagaatgcgcgctctgactgaagcttatggctttgtttctgttgcggcagaaggctttaaagctccgtctgtgattgtgaatttcacagatgatccggatatgaaatcaggtaagaagtttattgaagctggcttgcagattgcagcaggtgtgccgctagaaattggtgagtccccaaattacatgtcattccgcattggcttatttggtctggacaagttgatgaatattgaccgaacggttgacatctttgataaagctttaaaacagatcacagcccaatagccatatcaattatcacctcaattagtctgcgcgttcagccttgcttttggtggcgcagagagggcaggtccaagcgcgacgggtgggattatcctcttcacgcatggtggggacagtccagtaatagccacattgtccacagctcatatgccagataatttcttttgatacgctgattacaggtgggttcactggcggtgcatctgtcattggcgtgcgctttcatctatctttgtgggatacgcggtttctgcctcactgatgctgccatctatttcaagccttcgggtaaaacagcttcgcgtgccttcatgacaggcggctccagattggtcaaccagcaccaatatcgaatccgcatcacaatcatagcgcagctcaatcagcttctggatatggccggacgtatcgcccttgcgccacaaagagcggcgcgaacgtgaccagtaacagatccgtccagtggccagtgtctcagcaagactgtttttattcatccaggccatcatcagcacctcgcctgtgtcaaattgttgggcgatagccgggataagtccatctgccgtgaactgcaatgtttcaggtaaggggggaatatccgtcatgtcaaagcctctattgccgtatttgtaataattttcaacgaaattatctggattttccttgtgattcccttccttaataaagcaagattttcacattacacagcttaacatgccccctgaaaggacgctagaccatgtccctgaccttgcgcaaaaaagtctttatcaccgcggccttgacgggttcaggctgcacaactcccttcaaaccaccgctgaaagaaaatattgctgcccttgctgtcgcccatgctcatctgcctctgccagttggggctggccgttctattgggggaaggtagaggggaatttactcatgcctctgtgcagcaaactggtagatgatcgctggcgggcgatcagaatatatattccgtcgctggccaaaaaactgatgtctaatcgtaaatttgtcgggcagggcagaggccagctcaccctcacgcaatagaaaatcaggatttgacggccggccaaagatttcattgccctggccgaaggtctcatagagcagcattccgcctgggcgcaccagcgcgaagatctgtcccagcactgggcggtgtagatagtttgtgactagcacaaggtcaaatagcctattatccaacgcttctgcgagcacgagacccttgcgttccagatcaaggaagaccgccgttatttctttgcgcacttcaaggccagcagtcagcttttccaaaatctggcggtttgcatctacagcggttatcctgaagtttgcttcataccctcttttgcaaacagcggtaatatgcctgcccttcccacaagccagatcaaccagctcaacaggttctttggccacattttctggccgagctgccaaccagctgtcaatttgttctagaataaagggtgagggaggggtgggatcggtcatctcttgctactttgatttgcggggagaaacatttgctggccgtccagcgtaaaggctgctatctgctgctggccttcaattgagcttagccaatctgcaaaccgatttgccccgccctgatttacagacgggcaatgggaagggttgaccgtcagtaccgaatagcgatttaaaaggctgtcgctggtatcttcataaagcagcctgtggcgagatttattgccaaaagccagccaggtgctgatatcactcagcagataggcgtcgagttggacagccagattaatgctcgcccccatgccctgcccggcctccagataccagctaccgggcgcggaggagagattaatacctgcctctgaccataaacgacgttcacgcgtgtgagtgccgctgtcatctcctctggacaagaaacgtgattgtgagttagcaattgctctcaacgctgcagaaaccgttgtggcatctttgatgttcgccgggtcatctgccgggccaagcagcacaaaacggttctgcatcacctccagacgccgcgccccaaagccttttttcataaaggctttttcatcctcaggggaatggactagaaccagatccccatcacaattggctgcattgcgcaaggcttggcctgtaccaacggctaccacccgtacctcaataccggatttttttgtaaatgcaggtagtatcacatcataaagacctgaattcagagttgatgttgttgattgcaataggagatgccgcttatccgcagcagcaggtatcaccgctatcaggccccaaagagacccagccactataacggtttttatcaggtgaaacatagccttgcttagatacagcagtttcatggcactattcagtgtgataaatgttgaaattcaaattgtcctgaccacgcttaactgggcgtggccttatgattgcaatactacaaaacaacagtgtattgtcgcaaggatgattctgcggcgttcaaagagggaaagaacatggcgtcctcgctgtcaaatgctgaaccgaaattgtctgtcccgctgcagtcagaccaaactcataaggcccaacgcggggcaggtgtacgcctgaagctttttgtgggccaggcagcgattggcattggccagattgacttactagactgcatcggccggacaggctctcttgttctggccgccacccagatgaatatggatgaaacacgtgcagaaaatcttttagcgctgacctcggcgggcttctcaaaaccccttgtaatcccagccgggtcggacagcaatttacttgaactcacagaattaggcgctgctcttatccatcgttaccgtaagcaagacagctatatacagaagtcatcagctgatttgaaagactggctgaacaagcaacaggcgtcgaactgacacaactttctcaaagccctatcaagagtaaataaaggagagaacagctatggaattgccccaatggcatcaccgcccacaagtgaaacagaaaggtgtattggatcaagacgcttttctgcgtgtggcagatcagtttatctcgttggcgaatgacagaaacaaaaagattctggctactgaactgcattttgctctcatgtatgcagcggcacgctacacaggccatgtcggtaaaaatgttgtgaatattgaagatcaggataactggattactcatatgacggaacagtttcaggacatgctgcgcgaaaatatggctgatccggccctgtaatataaaaggctctgtgagatgatttattcacgccgcatatctgtctttattaaacacgcgctgtttgtcacaatgtgttttggcatgtgggttttggtcagtctgtttggtgtgatgcaggcccaagctagtgataaagttctgctggaaaagggcgaaaaacttgtgcgccagcattgcacacgctgtcatgtggttggcaatttaaataaatatggtggtatcgggtctaccccatcttttggggcgatgaaatccttgcctgattggcaggaccggtttgccactttctggtcgcttctgccgcatccgtctgttgtgcaggtggaagggctgtcgcctgaacggccaaaaggtttgctggctacaactaaacaaattttcctgaatatggatgacgtggatgccattcaggcgtatgtcgatactgtgccggtgaaagatttgggtggtggcgtcaattatcgctaacaggttgatgaaacttagcaaagttgaggagaaataaatatgactgacaaaaaacctgaaatatcggagatgagcgcaagtgaaatcgcgatgcgtaaatttttaaaacagctgggggtgaccacgcatcagaggttagaaactgctcttgctgggcgcataaaagatggggcggccgctgctggtgaccggcttgccatcactgcgcaaatagcaatcccggagttgggttttacacatgagatacaggctgagttgatggtgccggaggcagatgacagctgatgcaggccgttactgaagagcagattcttgcggctctgtccgccgtccaggatccctcacaaaataaagatattgttgcgttgggtctggtgcaggcaattcagatcaaagacagcaatataagttttatgctggtggttccgccgcatcgcggcccggcaatggaaccaattcggaaacgtgcggaacaggttgcgctttcgatagagggcgtgaccagtgcaactgttcttgtcacagcacatgaaagccgcgcagacggcctctccgctagttcatctagtggtggagcagcggacagacaggtgaaggaaaaaattcttgaaagcaaagttcgccggttcattgcggtggcctctggcaagggcggtgtcgggaaatcaacaacagcggtgaacctagctattgcgcttaagctggaaggcttgcgtgttggcttgcttgatgctgatgtctatggcccgtcacagcccagaatgcttggtgtttccggcaagccgcctgcagttggcggggacatggttgccccacttgagaattatggaataaagctgatgtcaatgggacttctggtgccagatgatacagcgatgatatggcgtgggccgatggtccagtctgctttaacgcagatgctaaattcggttgcttggggagaactagatgtcattgttattgacctgccaccaggcacaggtgatatccagatttccctcgcccagcaggtcaatctggccggggcggtaattgtttccacgccacaggatattgcccttctggatgttgtcaaagctctgactatgtttgaaaaagcaaaagtgccaatcttgggtatgattcagaatatggcagtttggcattgtccggactgtggccgtgttgatcatatttttggagagggcggggcagcggaagaagccagccgccgtggtattgaccttataggagatatccccctatctcttgctgtccgccagggatccgactcaggcctgcccgtgatactgtcagaacctcattcagcacatgctgctgcctataaacagattgcggccagtcttattgaacgggcagacttaagatcggaaaaaacgtcatgacactctattacgaaactgatcctgctaatgaaaacacagtccctcaattgccgcctctgctcagcgccgaagccgtggaaacgggtgatgtccaggccaaggcagtggcgcgtgctgcggcaggtgaggtcggactggtctgctatgcgacagctggcccgttacttgattttgctgtcacgctggcccctgaggtcgatgctgccacatctgcgcagatgcagcatgcgctgattgttgcggtgggcgatgcaatcggtgctttggccccacctgaagttgttgtgacctatcagtatccaggcacaattttctttaatcgcggcattgctggagtgattacaatgacacaaggcccggtctcgacagaaacaggctgcccgtcctggatggtggtgtctgcccgtctgcgtttgtctggcgagatgcaagggatgccgctagagttccgcatggaaaacacttcactcgaagaagaaggggcaggctttatttcgcgcacccgtctgctggaagcttgttgccgccattttctggtctggttacataaatgggaagaagaaggcttccgccctgttcatgacatgtggtcaaaccgtgcagaaaaacatgttgatttgcgggtggcagatggccgcacagcagaatggcttggcttagatgaaggcggtgccggccttttgaagcttgatggtgacgcggttgcggtcacacttgcggattctgcacaattatttgctgttcctgatttacaagacagcccttcatcaggagaggcggaatgaaactggcccgcacaatccgctttgatgcttctgatgaacatgtttttgcgcatgctgcagatgagggtgaatgggccatttctggtagcttctcctttgcccacaccacggatcaacaactgactggcaaaacaaaacaggcctttgcaaatggctttttggctgtgccctcttttggctattcaacacttgtctctgtggcaacagccagtgatgctgatattgcccagttgcgttctgcattggttgaacattttatctctgtttacggcgcaccagatgcgcaggctgcgggttcagccgcagatgaggaaattagctttatggcggagatttgtcatgaacataagacaggtacattacttagcctgcagcgcagcctgacagatgaaggcataaaagaacagtttcgtgccctggccaaggcggaatcatgtgcagagcagaaaatctggcagattgttgaagaggatgaaacagatgaacagcacaacagcggcacaaacgcctgatttttggctgacctctggctggcatttatgtgatcatgggccagagggtgggctgatgccgtcagctgattttatgttggcctattttcataggcctgaactggctttggtcgaagaatcctgcgcagcagaaacagccttgcatgaaaagctgatatctgatccttttgcggtggtaactgaagacgagctggccgtacttgctgatcctgatgtggcacataattaccgggcggttttggcgttccgtcagtttgtggctgagtatgatacgctgcaatctgcctatatggcgattgcgcaaggggcatctgtgtcctttccgcctttgtttgttgatcagatggcacaaatcatcctgcgtgaaattcttgatggcgtagatgaccctctgcaaatccgagctgcagaaattctttttcgcagccagtctgtgacaactgaggatggccgcatcatgatagcggatcagtccacggtccagctgcaggcggattatcagcgcagcctgaaacaacaagagcgcccggaagaagtgcagatagatatcctaacctcagagaccaaacagctctattgggaacgctctgaccgttttgatacctccgttgatatcgcgtttacccagccaggtctggatgcgtttgcgcgagtccttgaaaaatgggtggcacattttcttcatctgcaggtgaccatcacgccgttggtgaaaatagaagacgaccactggctttggcatctggggctggacatgaacgccactgccattctgaatgatctttatgctggcaaggatgtgtctgaagaccgcctgcgcgagattctgtgtttgttcaagctgaccgcagaaaccggcctaaagccggaaatggcgggtcatcctgtctatatggggctagctatgaacgctgcgggtatcgtgtcagccaagcctcaaaacttgttggtaaatctgccgctcagcgacgcgtgaggagcgccgagttgaaaactgattaccctctaaacgagacctgtgatcgcttcagttcgaccctcaattcagataatcatctgtggtgcgcggcttcggccgctcgccgacatcaaccatcttatcgccctggctaaacatcgcttctacccggcaatcttcacacattttaagcatatcagtccggccttctgcctggaacatgctgtggccagacagtttgttaatcacattttcaatcgatttggtggtgccaaaggctttaccgcagctggtacaatggaagggttcatcctcaattaccagctccgtggccatggccttatctgacaaattgaattgagggaccagtgtgattactttttcgggacaggttgccacacaaattccacattgcagacaggcatcttctctgaatagcaattgcggcgcatcagggttatcttgcagcgctccagctggacaagctccgacacaagataggcagatggtgcatttatctgtgtcaatatccacacgcccataaggtgcaccctcaggtaagggaataacatccgtctctgccccgttcgccttgcttaggccccttagaccaaggcgggtcacagcgcgtggactgcccatggcggcaaaaggtgcgggggtatatttgctagtttttgccttttgccaaaagatttgttcaacctcgtctgggtcactttcatctatcagcacaaaccggtttttgctgaccacacctgttcctgtcagcaacgcctctgccagttcgatctgctgatgcagactagcatattctggccctttgcgggggtccatcagcacaaaaatacgctcaaatcccagggttacggccccggccagcaagtcatgtccgaccctgccaacagaatgcatctcatacgcgatcaaggctgctggtaagcccttgccataacgggccagcatttcaatcatgtcacctccccactgtccgtcatgcaacagcagctgcgcagccgtcccgccagcttcgctataatagcgatgaagattagcaaggccggacagtgtggcatcgatgggtggaaatgctgtctgtgctgctccggagggacaaacggccccgcacatgccgcagcccccgcagacagccgggtcaatcgcaacatggtcgccagctactacaatagcccctgcaggacatacatcaaggcaacggctgcagcctgttaggctgttgcgcgaatgggcacataaattttcatcaaaatcaacatatatgggcttttcaaacacgccaatcatgtcagctgcttgtgcttgcacgcgcaacaggccggcccgatcatctgctgcacagcgcagatagccatctcgtttgtcccacccggtaaataagggcgtgccgcccgtcaaatcaatcaggatatcacagctggtttccaccccatctgtcttagctgaaaagacaggttctgaccgtccccagggttctgcttctgcaaagccgtcaatggtcaggctgaaagcggtgaaatggccgtctgcctttgttattttaccttgtgttagctggcctgcaaaagagtgagcgcttagctcttgtgtgactttgtccagcatcacggtcacgcccagtttatcagcaagggccttgcctaggctgagacctgcctcgccacccccataaatcagacagcgcccatgtgaggtgaacgtcatagaccgaacttgtggcactgcatcgcctgcactgcgcaacagggcagctatttttggtgaggccgaatcagcttcttctgaccagcctgccatctcgcgaatattgatagtctgcggcgcaggcttgccaagctcgctagcaatatcctcaaatgttttttcctcttgcgtgcaggcaacaagcaactgttcatccgcgcctatcgcttccatagccgctgttagtctgtcctgctgggtgcggcataaggatgtggctacatctgagccagcatctgcctgaccgcaagcctcgttaaccgttgtgccgtcaagcggcatactcttgccgcagttgcataacatgattttggtctgtggaattgtcatcttgaacctctgggcaaatggctcgtggtttgctggattacggtgcagcatcttgcagcaatgtgcagggatacccaacggcattttccatgatgtgatgtttcatgcccggttgttaaggggcaatctttggggaagaggttaaaaatggatgaccagattacggcgtcaacgcggcatgtactctatgcggtccgagtgattgttgagcgtcagccactggataacccgtggatcagccacaaatgggctgttcatgatttgattccgctcgatattagcgcaggtctaggtgcgctgccatccggtgatatcatactgcaacccctgcatgcggatagcagcgaactggacctttatatggctgatatccggattgatctgcaccatgctgaagctgaggcctatgctgaaaatcttcaatcttcagaccctgcgatttatgtagtgttgcgccgcaccgaagatgtggatactgacgaagaagaggatagtataaatggacaggcttgtgcagatgtccgcctttttgatgttagcctgtctccttataatattcaggattatgaggattgtggagaagatcagattgaaaagcttccgcttcaagggccgattgcccagtttgtcgaatcctttgtcgaacagcattttacgccggaggtttttgtcaaacgcaaacgggataaggcccgtattgatgctgatacccagcgtggtggtgatgcgcgtctagtgcgtcgtcggacccattaaccagccttagcactggataagaacaagatgagcgagacagagcaaaaaccttttctgagtaggtgggcaaagcgcaaagcagccaagcaggcagaagttgatgtgctcgaagaggttgcgcagacagaacaggttgacgcgacaggtgagacggaagaggaagctgcgctgtcggatgaagagctatgtgcccgctatgagctgcctaatcctgaaaactgcacagactcagaacagctggatggattttttgacggtcagattcctgacaggctgcgtcagcttgccatgcggcgcttgtggcggctgaaccccttattcagatttgcagatgaaatggtcgaatacggcgaggattttacagatgctgcgacagtaattccaaatatgcagaccgcttataaagtgggaaaagggtacatggataaattgctcgctgaacaggaacaggctgatgcagaggccgccctttcagttgaacaggtggcagagcaagacgaactcaaaggcacaggcaatacagatacatcagaacctatagaagatggtagtgaggagacaacggcgctggctgaatcaggccaacaggaagatcctgatcaagcagatgatgccgaaaaggatagggataatgaagagcaagtaggggctaaagatagctccgtcttggctgctgaaaagagatctgacgctgagcaacagccatctgaaccggcgattcggcctcggcaaatggttttcacaaacgtcaaaccagcacgatcctcataacgagaatgactctcaaatatatgtgagattttttctcaaaacaggtcttttttatatgttttagctgtatttcgtgagactcattctcaataagaagcgaagtcacaggaatattttcatttagtggaattaatattaacctaacccctggtcagacgatcaattttccgctttactctaaagtaaggggaaatcaattttgaccaataatgccgtaaaatcggaaagacaagtggtagtcagcgaagaggatgaccttcgtgcccagctttatgagtttcttgcaacactgttaagggtggagccaacggacgctgtagtaaagcaggtggtcaatttgtcaggagatgatactccaattggccaagcgtcatccactcttgcccacctggctcaaaaaatggacggaacaagtgtacgaaacgaatacgtggacttgtttattggtgttggccgcggtgagcttttgccctattgctcttattatttaactggttttcttaatgaaaagcctcttgcgaaactgcgccaagatatggcggcaattggcattgctcgcgcagatggggtgaaggaaccggaagatcatattgccagcctgtgtgatatgatggcgggtcttattaggggccaattcagccgctcttttacattggctgaacaggcatcattcttcaaaaaacatttagccccctgggcaagtttattttttaatgatttggaatcagcgaagaacgctgtcttttacgcgcctgtcggctcaatcggcaaggtgttcatggatatcgaaagcaagtctttcgatatggagctgtctgggtagcgaatggccccgggcaaaacagccgcgctggcaagacttgatttggcagcaggcaacattgttggaggaaaaacaatggaaacaaaaaagcaggatgttgggcaaactggtcgccgtgactttctgaaactgtcagctcttggggcggcagcagcaggaactgtcattacggccggaagtgcaacagctgatgaagcaaagactgtatcagggtcaggttaccatgaaacagaacacgtaaagactttttacgaaacagctcggttttgagcaagtttttgaaaaagtttaatcagtcttaaccaagtttaggaggaagacatgctcaggaagaagacgtcaggggttgcgaatggccccctctcatcatctttcattggcaaggcagccgaaaaggttgttgatcgccgtgcgtttttgcgcggttctggtcttgccattggcggaatcgctgctgcatctgcgcttgtgggcgcaaatgtaaagccagcacaagctgtaatagcaggtggtgcagcggaaataaagaagactgtgtgtactcactgctctgtgggttgctcagtgatagctgaggtcaacaatggtgtttggactggccaagagccaggctgggacagcccaatcaatttgggcgcgcattgtgcaaaaggcgcatctgtgcgggaaaccgcttcgggtgaacgccggctgaaatatccaatgaagctagtcggtggaaaatggtctcgcatgagttgggacgaagccatcaatgagattggcgacaagatggaagacattcgtggaaagtctggaccagattcagtatactggctcggttctgcgaaacacagtaatgaacaggcttatttgttccggaagttttacgcttattggggaacaaataatggagaccatcaggctcgtatttgtcattcaaccacagttgcaggtgttgcaaatacctggggctacggcgccatgacaaactcctataatgacatccacaattcacgtgcgatattcgtcattgggggaaatcctgctgaagctcacccagtatctttgcttcacattctaaaagcaaaagagcagaataatgctcctttgattatttgtgatccacgtttcaccagaacagccgctcatgctgatgaatatgtgcgcttccgtcctggaacagacgttggtctgatttggggcatcatgtggcatatcttcgagaatggatgggaagataaggaattcatccgtcagcgtgtatatggcatggatgatgtcatggatgaagtgaaaaaatggactccagaagagactgaacgtgtcacaggtgttccaggttcacagttgaagcgtgttgcacaaaccatggcgaataatcgtcccggaacgtttatttggtgtatgggtggaacacagcacacaaatggtaataacaacactcgtgcctattgtgcgcttcagcttgctcttggaaacatgggaactgccggtggtggtgccaatatcttccgcggtcacgataacgttcagggcgcaacagacttctgtgttctctcgcactcactgcctggctattatggcctatcagcaggagcttggaagcactggtcacgtgtctggggcgaagattatgactggatcaagggacgttttgatacaataaagggttctgatggcaaagagaaaagccttatgaacttgaagggaattcctgtttcacgttggattgacggtgtgcttgaagataaagagaacatcgatcagccagacaacgttcgggcaatggtgttctggggccacgctccaaactctcagaccagacagctagaaatgaagcaggcaatggaaaaattagacctaatggttgtaattgatcctttcccaacagtatctgcagttctttctgaccggactgatggtgtttacttgttgccggcgtcgactcagtatgagacctacggttcggtgacagcatctaatcgttcattgcagtggcgtgaaaaggtaattgatccgtcatttgactctctgcctgaccatgtaatcatgtataagttcgcaaagaaatttggctttgctgatcgcatgttccggaacatcacaattaacggtgaagagccgttggtagaggatgttacccgggagttcaacaaaggaatgtggacgatcggttataccggccaatctcctgagcgattaaagctgcatatggagaaccaacacacctttgaccgaacaactttgcaggctattggtggaccagctgatggcgactactacggcttgccttggccatgctggggtacggctgatatgggtcatcctggcactcctcttttgtatgatacatccaagccagttgcagaaggtggtttgtgcttccgtgcccgttttggtgtggaacatgaaggcaataatctgctggctgaggggtcataccctgttggttcagaaatcaaggatggctatcccgagtttaatatggccatgctcaaaaaactgggctgggacggcgatttgtctgcagatgaaaaagcagcaatcaaaaaagttgccggtgataagacaaattggaagactgacttgtccggtggtattcaaagggttgcaatcaagcatggttgtgcgccgtttggaaacgcaaaggcaagggtcaaggtttggactttcccagatccaattccgttgcatcgtgaacctctttatacaaaccgtcgtgatctggttgccgactatcctacatatagcgatagaaaagcatatcgtttgccgacattgtataagtcaattcaggatgttgaccactcgaagaattatcccttcatcttaacctctggccgattggtcgagtatgtaggtggtggcgataatactcggtcaaatccatggttggctgagcttcagcaagacatgtttgtcgagttgaatccaagggacgcgaattcaaagcgtatcagagatggtgatatggtttgggtaagcacaccagagggtgctcggattaaagtgatggctatggtaactgaacgtgttgcggctggcgttatgttcttgccgttccactatggtggtcatctcgagggcaaagatctaaggtctaaataccctgacggggcagacccttacgttttaggtgaggctgctaacacagcaatgacatatggctatgactcagtgacacagatgcaagagacgaaatgctctctgtgtaacattgagcgggcttaagggaggtttgatatggcacgtatgaaattcctttgtgatgcggaacgttgtatcgaatgtaacgcctgtgtcacagcgtgtaaaaatgaacatgaagttccatggggagttaaccgcagaagagtggttactattcaggacggttcaccaggcgaaagatctatatctgtggcctgcatgcactgctctgatgcgccatgcactgcagtctgtccggtagattgtttttaccaaacagaccaaggtgtggttcttcactcaaaagacctttgtatcggttgtggttactgcttctatgcatgcccgtttggcgcaccacaatatccgcaagcgggtaactacggttctcgtggaaaaatggataaatgtacattctgtgcaggtggaccggaagacgacatgtcggaatcagagttccaaaaatacggacgaaaccgtcttgcagaagggaagcttcctgtttgcgctgagatgtgttcaaccaaagctctgttggcaggtgatggtgaccaagtgtcaaacatcttccgtgagcgtgtggtattgagaggctttggctccggtgcttggggctggggtacggcatatcagcaaaagtctggctaagactttaaaaaaatgtttaggggcgtgctcagtttacaataggtaaaactggcccgcccctaaatttagaatttgaatttacaaccttggagttgatcgcgtcatgttaggaaaactaaagaaaaggtactttttatatgttcctatcagttttttgtttttgcttggattgactgcatgtcaggaatcggagcgagatcgtgtattgcgatatgacaagggcacttatttgggggctaaagatcaaagcttgaatcccgagcaattacaatcgttacagtcgcgggcgaggtatcaattttcgtcgtaactcattgttaaatgttggtctaataggagcattgaaatgttatccagcaatctaattggaaatagggtaataggttttacgttggccatattgttggctttcttaactattaacctcaacgttaacccagcagccgctcaatcaaagggtgaagtccctggtcaagctctgggtctcaattctgacgctgacctttggaggtttatcagaaacggcaatagtggcaatacgcaacttaaggatgaattagccgctgtcatgattcagtcggagggtgataattggagagctgttcgaaatgggcctgtatcggtttatggcgcaattggtgtgattgggataatagttttattggcaggattttacagctggcgtggtcggataactattgatgccggaccatccggcaagaccattgaacgtttcggtacaattgatagatttgcgcattggttaatggctggctcatttgtaattttggcaataacagggctcaatcttctgtatggacgttacacattattgccactcttaggtccggaggcgtacacagctttctccatagcagggaaatatgcccataattatctttcatttgcttttatggcaggattggcattgtcctttttcctctgggttaaacacaatattccgtctaaggtggatatagaatggctgaaagctggtggagggttgtttaaaaaaggtgtccatcctcctgctagaaaattcaacgccggccagaagattattttttgggcagtaatgctgggtggtttttcaatatcgttatccggcattgctcttttgttcccttttcagactgctatgtttgcgaaaacatttggggttttgaacatgattggctttgatctgccaacagctttgacagctcttcaagaacagcaacttaaccaactttggcatggcattgtatcccttgtgctcatgacgattattgtagcacacatatatattggttctgtgggaatggagggtgctcttgatgccatgaattcaggtaaggttgacaggaactgggccaaggaacatcacggcctttgggtaaaggaagttgatgaagcagctaaaacagcgaaagccagtcctgccgagtagaaaattagggcaggtccggaggtctgatgccagacagcagacaccggacctgacgtttgggatgtccactcgtgacgatttttacagtgattgatagaactgcccgcagcaagacagccggacttctggctagcttttgcgggcttttctgtttgctgactggtactgctgtcgcgcttgagcagtacacagctcatggcgggccggtgaaacatctaaccctatcccctgatggcagcctgatggtctcatccagctttgactattcagcagttgtgtggtctgtgccagagctggcagatgaagccacgctaataggacatgatgcagctgtgaatgtggcacaatttacacctgatggggattggctagtaacagggggtgatgacaatcaaattctcctttggtcagtggctgatatcctcagtctgggtgacgaaacaaccccctttgtgctgcgaggtcattatggaaaaattgtagattttgccttttccagtgatggtagatatcttgtatctgcgagttgggacggcattgtaggcctttgggacatggctctagcccgtaatcgcttggagcaaattccaatctcgtttcgtggacatgacggaccagtaaatgatgtccagtttagcgctgatggcaaacacctttttagtgctggcaatgatggccatatacgctattggcgggtagctgatggcgaatatttacgctccattgtccgtaacggctggggtgtgaatgtaatgttggtagatgaggaacgaaatattttggcttatggctcaacagacggggcgatggtcttagccgcgctaaatacaggagctgaatatttgcgaatgggcgaagaccgagtgccagttctatcagttggcctaaactccaccgccaatcagattgcttttggtaatgccaagggtcaacttaagattattgatttgaataccacgacgttgctacgtgatttccgcgcagcaaatggtccaatttggtctgtgatgttcatgccaaataatggtgatatgatgatcgctagtcttgatgattttattacgaaatggcagattcatgattttccgcctgaaattttggatacacccggtccaacgcggaggtttaaagtaacctctgaaatcagtaatggtgaacggcagtttgcaagaaaatgttcagtttgccatacgcttgttaaggatggagcaaaaagagctggtccaacattatataatatttttggtcgtgaagcaggtaccttgcctggctataaatattccaaagcgctcttggactccgatattatctggggtgaagagactattcacaggttgtttacagatggaccggatgtggtaacaccgggcacaaaaatgcccatacaacgaatgaaaaatgaagcggacaggcaagatttaattgcctttttgaagcaggcaacaacatctttaaattaagagtcagggagagagatgatgaaaatgtttttaataagctctactactgcagtagctctagctgtagtggcctattttattcttactagcataggtatggacaccgcatctgtgatgtccggttcatcagttcggctttgacagcaagattgttttcttggttggcgcctgttgttgtttttctgacaacggtgctctcttcccttgcacaagctgaccacggccctcatagcttcaaggccattgaagcatctgttctgcgcgtgctgccaacctggcctggctatgagcggccaggcttcggcgcgccactgggaacagcgccagaggggacaggctttgttattgccgcaccctccagtgcacacatttctggaagctctcaaaaggctagcagcccatatcttctcaccgcggctcatgtggttgatagtgccacccgtattgaaataatgagttcgaatggtgtgcgggcagacgctgttcttctagctgtggacccccaaacagatctggcgctgctgaagatgcctctggccttgccgccagtcaagttgatgctggatacacctgctgtcggcagccatgcttgtgcagtggggaacagttttggtctgggaattagccttagctgcggtgttgtctctgcaacaggccgcaaaaatataggttttaatcctgtcgaagactttatccagacagatgcagctgttaatcctggcgcgtcaggcggtttgctaataaattcagctgggcaaggccttggccttattgatgcgattttcacaaaatcagaagacagtgacgccggtgtgaattttgctgtgtctgcaaggctaatagacagtgttttgttggcatgggcagacaaaagtgatgtttttacccattgaaatagttgtatactggtgacttgcatctgagtgagaaggtcacctagtcttatgtacttgattcgggtttaggaaaaaagggcgcatcttaatggatgattttctgaaagttacttcaacaagagccagggatctaacaccagagccggtcccatcacagctggttgacccgtttggccgagcggttgattatattcgcatttcggtcactgacagatgtgactttagatgtgtgtattgtatggcagaggaaatgacttttctgccgaaaaaagatctgctgacattagaagagctggaacaggtttgccggacatttatgtcgatgggcacccggaggatcaggcttacaggtggtgaaccccttgtccgccgcaatatcattcaactgattcgtaatctgggtgcagaggtgaaatcaggtcaactggatgagctgaccattaccaccaacggcagccagttgacaaaaatggcagatgatttagcggaagctggtgtccgccgcctgaatgtgtcgattgatacgctagatgcagacaaattcaaactaatcacccgctggggcgacctagataaagtgcttgacgggatgaaagctgcctgtgatgccggtctttcgataaagctcaacgctgtggccttaaagggcgttaatgatgacgaactgggtgatatggtcgcttgggcaggcgagcagggctatgatatgaccattattgaagtaatgcctatgggggatatcggcaatgaaaccagggttgaccagtatttgcccgtttctgtggtccgttcacgattagccaagcgctttacactgactgattcagattatgccagcgctggacctgcgcgttatgttaatgtaactgaaacaggccgcaggctgggctttataacaccgctgacccataatttctgtgaaagttgtaatcgtgtccgcctaacctgtacaggcccagctttatatgtgtctgggacaggatgacaatgctaatcttgcccgcgctctgcgtgaggatggtgaggagggcctgcgctcagcaatcatttctgcgattgcgcgaaagccaaagggtcatgattttgtcatcaacagacgtgaggcgcaggccgttggccgacatatgaacgtaactggcgggtaaaaggccgcctagattacagctgagatgacagacacgataacagatgcgataaaaggaacgcccgtcgctttatttggcctggctggctggtcgggctcgggtaaaacaacattggcggaacagctgatcacagaatggacggcgcggggtcttgatgtagcgactattaaacacgcacatcacgaatttgaagccgatacgccgggcaaggatagttggcgacatcgcgaggccggtgcgcaccaggttcttgtttcttcagcgatacggtctgcgcattttgttgaacataaaactacagaacctgaacttggccagctgcttaataggcttttgccttgtgacctggtgttgatagaaggcttcaagcgcgaagctgtgccaaaaatggaagttttccgtgctgaagtgggtaagcctcatctctacacagcagataaccagattatcgctgtcgccagtgacagtgcggtaacagactgtcctttaccagttctggatttaaatgatataccgacgattgccgattttgtgcttcaggtagttggcctcagcagcaggtagggagtatcatgccacatcccttttcaactccgctgaatattgcccgcgaacagctattgtccgcggcgacaggctgggttcctcaacaagaaatctgcggagtatcaaatgcgcttggccgagtgctagcgacagaacaaataagctctgtcgacctgccccagacagataacgtggctgtcgatggctatgctgttgaagcagcttttttggccgcgcatcccgagcatttgttcaaagttgctgggactgcacgggctggtcatcccttttctggcatagtcgagccaggtgatgctgtgcgtgtctttaccggggcagttatgccagaagggccagattgtgtgattatgcatgaggactgccaaatatctgatgttggaattatctgcggtaaacaattgaaatcaggtaccaacatgcgttcggctggcgaaaatatcgctgctggggaaacgcttgccgcaaagggggtctttcttggggcggcaaatattggccagttggccgccgcaggcctcagccagatacccgtattccagcctctgaagattgctttgatgtcaacaggggatgagttagttcaatcaggtgatttgcgaaaagtcggccagatcttcgacagtaatcgaccgatgctggcctcgctatgtcaacaacagggtgcacagctaacagattatggcattattagagatgatcaagtcagattgacttctgcctggcgagaggcgctagaccattgtgatgtgattatcagttcaggaggggcatccgacggggtggaggatcacactcaggctgctttaaaggcatgtgaggcagaatgcctgttctggcggttggctatgaagccgggacggccgatggctgtaggcaaaaaagggcatcagtttattttctgtctgccaggcaatcctgtggctgcctttgtatgttttcggcttttggtgcggcctgttctggacaagctgtcaggcgctcatccacgcctgcccttcagcctaatgctttccagcggctttagtcataaaaaacaggcagggcgggctgaatttctgcgcgcttgcgtaactgaaactgagacaggtgtgcaacaggttgtcttgcatgggcgcaaaggtgcaggtgtcatctcgtcattgacaggggctgacgggcttgttgaaataccccttgccacaacagagattgcctcaggccagcctgttcggttctatccttttcatgaggttgtttgatgaatattctgtattttgcatggatgcgtgaacatactggttgcgcagcagaagaaattgacttgccagatggcgtgtccactgtcgccgatcttgtgccgcatctggctagccggtcagacgggcatgcaacagcgttgcgaaatctgaaaaccgtgcgggttgcagtgaaccggacctatggctcacttgatacgccgatttgccaaggcgatgaggtagccttttttccgcctgtgacaggaggctgaactgatgattgaacctgaaaactgtgctgctgcggatgtgagtattagaaccgcagactttgacgctggcgttgaaattgacaaattgcggcgtgctggtgttggggcgatagccacctttagcggaattgtgcggaatgacgctgaaacccctggactgattgcaatgacactggaacacttcccgggtatgacagaacaagaaattcagaacattattgatcaggcccgtcgccgctggcccttaacagcggtgaaggttattcaccgtgttggccgacttctgccccagcaaaacattgtttttgtaggtactgcttctgcgcatcgccaggcagcctttgacagcgcttcttttataatggactacttaaaaactcgtgcgccgttctggaaaaaagaggaaaccagttcaggcgtacagtgggttgaagcacgcgaaacagatgatattgctttgcaaaaatgggtgaagccacggtaaaggcgcttcactctcaattcataatgcggaaagtgggatggttatctcattgtggatgatgataaagtggaggtctcggaaagtaggaaaaccgagacctccatcggggggaagactttttcaagtcgtactttatacccacgcaaaaccgatttagattggcgtgggtatattttttttgaattatagccacataattctgaataaaaccagcctagattgcagaattgcaagtggaattggtcttttattcccttttgaaggcttttatagatgattttccggctagaaacaggcttaagcgcaattgctgttgcacggtatacagcaagtcgctcaactgttttggatgcagatgaccagcgttatcttctactgtacgcaggatatgacctgccatgccaactaaatctgctcccaaccacaagcatttcgccgcatccaagccatgccggactccaccagaggcaattatacacaggtggttggatacagcccgggcctgcgcaatcgcatcaggcaacatcaggccgcaggataaaaagggcgcataatgcgctcggtcagtttccgggcgtctgttaagctcaatttgggcccagtttgtcccgcctctggccgcgacatccacatgccgtacaccaatggctgccagccgtcgcactacattaccagacaggcctgctcccacctctttaactagcacaggacaattcagcgtaccaacagccgtttcaatcgccgataataccccgcgccagtcatgatcaccttccggttgaatggcttcttgcaaggggtttaaatgaattgctagcgcatcagctctgatatcttcaacagcagcgcgggccagcttcagtccgtcctttccagcgagctgcgcgccgcccaggtttccaatcagaacagcatcaggagccagacggcgcagctcagcctgactttgccctgattcaaggctggcacgttgagatccaagtccaagcgcaacctttttcttctgggcggtatctgctagtaccctgtttatcgccatggcccggtctgtaccgccagtcatgcctgttattattagcggggaatccagctcaattccgagaaattctgtggtaaggctgactctgtttaaatcacattccggtagcgcgcaataaggcaggctcaccgcatccagcgggtggtcggcgaggggctggctggtcttggccagatcaagatgcgcatttttacgatcagttgttagcggcgtgcttgttcgggtcatggctgtgtaatctttttcaatgtggggcggtacattgtatccaccaacaacatatgcggtaccgtcaaagcagctaacaggataaagactgtgcggataaaggcttcatctggtgaaaaactgcctgacattgtctggctaaaatatagccctgcgcccatcgcccaggatgctgcggtcagaattccgccgctgattagaatgaagcggcggctgacaaagagctgcatagctttccagatactagtaaaatgacggcgtgaatgtacaatacagaaataaaccgcaaaacctgctaagggcggcaatagccacactagcacagccagcccagccagttcacctgccgcagcagcatattggcgtttgaaaagggctgcgctagcgtaaatacaggcagctgcaagccagattttcagcgctggtctcagcgcatcaatgatcagaaccgcatttggcccggcaagaacaataaacagttggctcacctctgttgtgtgaaacacaggcaataaaatagtcaccatgccaccatgcaccagaatctgacaggctttcaggctgcgctggacaacgcgcgggcctggttgaaacagatgttctgtatcgccgctaccaaaatgcacaatggttagcaccaaaaaaccagctagggcgaagacaggtgatacaagccagatcaaggcagacaacccggccaataacagatacatgaccagaaaaccaataattttgccaggactgcgcccaaagcctaaacaaaaggcaagggcgccgtcaaatgcaccatggggcaaaccaatcagcatcaccgctgcaagggcaaataggtctgttactgataagatatctatcatatttactctgaatctacgccgtctagaggcgttttgctcgcattattttttatccgcgttatctctgttggtcacatgtatattccacaatgcgtgtaaaaacaggccggctggcattgccgtgatcagttttgcatagattttgaaatctgcaaggccagacataaagcgggcgaattcatcgccgttcagggcaagggcgatctgcgcaaacaactcagctgccttttcagggtgatgccggatgacgtgcagaaagattctgtccagaaacagatcaaaacgactgattggtcgaggcattgttgctggcgttaaggggtctgtaccactgaggaccagtctgtcaataatagcatctgtatgtttttggataaaggcaaacgcatagccagatgacgggcgaacacaaccgccatttgcaccgatgggcagataatcgagattgactggttgtacaaaagccatcggaatacagccctgctcagtatgcttgacttgccagccgccagtcgcccaataagtttccaaataggtttttatggcctctgcataaaagtcatcgttctgccgtttaggggaaagcatggtagattcgactaatgcctccgtagggctgtaaggaagcagatagatgaaatgaatgccgcgggattggtcacatctgaaatccattaaaatagccgtatcaggtgaaaaacaaggttgtgctgctttgatctccacacccttaaaatgctgcagcaaggccccctcaggcacatttggagggcggctgtcaaagacaggttgggcgccgggtgtaggcaggttgtcaggcttttgctgaatattaccgtctataatgttcagacaatgggtcagccaatcacggctatcgagacggcagtagggatgagaatcagctgtttgcgtaaccttccgttctgctgtgataatctgccatttatgccagcgttgtcttgagagctcggtctgctctttaagtccctgtgttgcccagaatccccaactatgcctatttgttgcggcgggagagacaggaccatacagacaagcctttataattgatgtcttttctgtccgcaccagctggcgggccaacgacaggccggcacagcctgctccaataattgccaattctgtctgttctagggaggctgttctgatcatttaagtgcctcatctaataaactggtaagaggcgcatgcaaattggtgttatgagtagcttttggcaggcgatagagtttggatagggcctgcaggctagcagacagtttctcgctcttgctagttaccacgcgcccatcgccccaccgcagcttacgggttcttaattttgtcccaatggcgcgataaacacctgctgcaacagcaatgcaatatcgtgcgcgcacaggcagaaatcctagccccagacgcccgctttcataatactggtcagcaagagtcagcagcctgtctgcggcagcttgaatctgcttatagttctgcgattcaggggcctctgcacaggcaacaatctggtcaggcgagagctggcctgtccagctttcaggcaggtagcgacgtcccatctgggcatcttccagaatatctcgggcaatatttgttagttgcatgccgatgcccatatccacagcaaaacggaaggctgtcctgtcgtcacagcccagaacaggacacatcagaagcccgacagcgcctgctacatgataagcataagccaccaaatctgcctcatccttaagcagcattgcagactgatccagcaacagcccgtccaatagatggataagtggcataaccgggatctggcatcggttcataacctgcatatactgcaaaagtgctgggtctggctgttcaggtattttcccagcatctatttgggtcagttggcggcggatatggcggagtcgatgtgttccttctttcgtatccagcccgttgatatcgccatcggcaatatcatccaaaagccggcacaggctatacagctcagctgcatctattgccatctggcggcccagcaggcgtgatgcccagtaaaagcttttgccatttttagccagtgtctttagggctgtgcctctctcagctggataatgtgcgtcttgctgtttctgttcagcgcgtgggctggacatgtgcggctttccgttcatcttcgctgaccaacgtttcaacgactttggctgaacagagcacgccaggcataccagcaccgggatgtgcacctgctcctgaaaaatacagatttgccagatgcggatcacggttatgataccggaaccaggcagattgggtgaagctgggggatatagaaaagccagcaccatgttcagatcggtaatctgctttgaaatcttcaggtgtcatataaaaatcttcggtaatcacattacgtaggtctggcataatacttttctcaagagcggtgacaatcctgtcttgcaggagtggcccttgttttgcccagtccacatcaccctgcaggttagggaccgggcagaggacataaaaactatcacagccctcaggtgcaaaacgggcgtccgtggcggtcgggcgatgcagatataatgaaaaatcttctgccaatatctttttgctaaagatatcatgcagtaaatccttataccgttttcccatccatatcgtgtgatgggcaatgtctggatagcgctgctgggtcccaaaaaacagcacaaacaggcccatcgaatagcgggttactgcctctggcagcacctttttgcggaggtgggtctggctaggcagcatctgccggtacacagttggtgggtcagcgttacaaatcacattatcagcagaaataaaggccccgtcggccgtgcgcacacctgtaactttcttgtcagaaacacaaatctcaatgatatctgtattcagtttcagatcaacaccctggcggatcataagtgctttcaattcagcaaccagcttgcctgtaccgcccatgcaaaaatacacgccccatttgcgctccagataatgaatcaaagcataaatagatgtcgtatcgaaggggttgccgcctacaagtaacggatggatggaaaaggcctgccgcaataaggggtgtttgatgtggctgtttaccagttgggctactgtcaaatagctgcgtagccgcaataattgcgggattagcttgatcatagaggtaaaacgggtaaacggcttatctgccagttgggtaaaacctacatcaaaaatctgtttggacgtgttcaccaacctgtcatagttttttgtatcttctggcgaaaaccgagcaatctctgcatgtgtgtcagctagtgacgggcggtaatcaaactcagatccatcatggaaatgaaacctgtaccatggatcaagcggcacaaatttcagatgttcagaacgcgtctctccaaacaggtcaaacagctcatcaaacaaaaatggggcggtgattacagttgggcctgcatcatggcgaaagccgccacgctcaaatacacgcgcgcggccaccaacatcttctagccgctctatcagcgttacctgatggccttttgcacgcatacgcaaggctgcagcaattccgccaaaaccactgccgacgaccacgctatgaacgcgctctaccatatctttcttataggagtaggggcgggcatcatccatttgccacatcctcattttctagccggtttcctccagtctagcacaggatatgcctgtctgtcctgcccaaacattttattggatatcacttaggcatttaaactggtaagatcgcggcagacatgccgaagctgtgccaatattggtgtgatgattgcccggcaatctgtgggaaaggcagccgatgatgcttctgcttcttccagcatgctcagcaaggctgatgaagtcatggtcagcgctggtgaccggcgcacatgttcctgccaggtaagcgcaccatgtgtgtcgcctgaggacagccacttgtcaaaggctgtcgctgtatgcttatccaatgttgtctgaaacatcacaatcaccgcgttcggggcgcgacgcagcaggtcggatgccgggctttccgcgccatccttaccaatcacattcaacatatcattggcaatttgataacaaccgcctgcagcactgaaaaaccggttcagcggccgtattgcatcacggcggccggccaggtgagcaatcccttcaaccgatgcaataaataaaggggcagttttgcctgtgctgatgtccatatagccagaccagtctgggtaggcgcgaacctcaaactcgagagcttgtcctgccgtggtggcggccatgtggcgtgacaggatagaaaccagctgtggcgtctgcccgagataggcagcttctgctgccagctcaaaggagagcgcaattagccagtcaccaagcgccagtgccgtatcgcgcccataaaatgcccagacagacggcgcgccacggcgcaccatatctccatcacagatatcgtcgtgcaccagtgaagcattatgaagaacttccacagctgcagcccagggtaggctggccgttcgtggtacgccaaaactgtctccagcggctagagccatacggccacgcaactgtttgccagtacgtttgaaatgatgttgagcagcagtggcgagtgggtgtttgcccgatggcaatctccgcacaataaaggctgacagtacagcagacaaatcaggaagagcatcaagcttttctgcgccctgcatctcattgacttggctggctatgttctctgtcatcttccggcccttcttttgtagatattttaagatgaaaggtgcaaaaaaattgagaaacacataattaagctgaataaagcgcaagcacccatttaaattatggatgcttgcgataagtttattactttgccgcttcagattgtgaagtagcagcagcccaaattgctaaacagaaaccaatcttgttaacaacgtcagcaacgttgtagatcaggttcagcgaatttgcgtcaactcctccggtcatgtagccaaagaaatatccaagtggataaatagcccaaccaatagtcacaatcagacgcattgtgttgaaagctgattgtacagatgggggcgcaccttctgcagcaaccttgccagcttcaccagcaaagatttcatacaatatatatccccaaccggccattccgatcacgaaaccaaccgtagcgttgatgaagcccgcctcacctagatatccgccaattaacattaccattgtgccgatcatgaggcgccagaagacgccgacagcaatggcagtgcaagcagctaaaataaggtagaattcaatcatctgcagaggcactgtgattagccaatctacataacgaaagaccgtgggcgatgcacctgtggcaacccaaacatcacgcatataaaagtagtggaccgcagctactagtgtaacaagtccgcccacaatcattgatgttctccacttgccgtcaactctcaatgattccatcaagaaaaatacggtagcggcgaccattgcgatcgatatcaaccaaaaggtaatgccgacaggatcgttcggctccaagtttccaccagctgctaatgcaattgatggaatgagaatagccagtgatgcaatagcgactgactttaaagacttaattattgacataggacactccccaaacgtcgcgttctagaacgtcct

SEQ ID NO: 2 - DNA, 40220 bp, Unknown
  uncultured marine microorganism HF10_25F10
ccgacatgtatcttaacaaggacgtctacaactcgctgacggaccattagaagaaggcattcgaggttgccgcgaacgcatctctcatcaagggcatgtccgcacgtatctatgacaacggccaggcgctgcacgagctgaccaccaagcatggcgtgatccttcaggacactccagcagactacttcccggcatacatgaatgccgcgaacgcgctgctcgagaagaactccgcagaaaacgagttctttgctgaggtttggcagtcgcagaaggatttcgcacgtatcgcggttccgttctggtcgggtgcacagagctcgaacgctcagcttggcatggcatatgccaacagcctgaagtaaatcattatggcgtgaaccgtggcttgctgcggttcacgtccttcttggtcacttttacaaccatccagaaccggcagcgcggcgggggcattccatggcacaggaaccagagatcgaaaagctcgagattgcagacgagctgatcgccgaacgtcgcagcggcaaggtcggtgacacgcccgtggacatgcatcccctgatgcgtctgatcgtcggcaatatcgacattctgaacaactgggtcggcaagctgacctgcctgatgcttgttccggttatcttcgcgatgatctacgaggtcgtcgcgcggaaggtctttatcgcgccgactgactgggcctacgacacaagccggatgttctccggcgcgatgttcatgctgggcgccggttatgcgctgatgcgcggggtccatatccgcgccgatttcctcttccgcaactggtcgccccggacccaggcgctggttgatggcgcgctctaccttctcttctatttcccggcgatgctgttcttcttctgggtctcgacggaatacaccgtcaaggcatgggtgacgtgggaacgatccatggatacggcgctgatggcgccgctcgcgccggcccgaacggcgatgcctgtcggtgccctgcttctgctgcttcagggcgtggcggaattcctgcgcgcctatcaccagcttggtgccggatccatccagaaatgggtgcttcgcctgcttcccgtctatgtggtcgtgctcgggatgatcttctgcaacagcctgttcccggggtttttcaactttgaaatgatcttcggcgagtcgttcgacggcggcatcaagggcgccggcggcgtgtcgccgccgatgatcggcgtcatcatgatcgcggtgatgctgttctcgattttcattggcttcccgatttccttcacgctgattttcctgtccttcgccttcggggcgtggggcttcggcggcaagatggtgttctacctgcagaccctgctattcaacaatgtgatgctcgagcagacgctggccgcggtgccgctgttcgtattcatggggatcatgatggagcaggccggcctgatggaacgcctgttcacttcggtgcagttgatgctctcgcgcacccgcggcgcgctttacctcgcggtattgttcgtatcgaccatcttcgccgcggccaccggcatcgtcggcgcctcggtgacgatcctcggcatcatggcggcgaaaacgatgacccggtccggctacgacctaaggctggcagccggaacaattaccgccggcggcacgctcggcatccttataccgccctcgatcatgctcgtcgtgatggggccggtgctgcagattccggtgactgacctgttcgctgcggcgattgttcccggcatcatgctcgccggcatgtatgccgcctacgcgctgatccggtgctggctgaacccgtcgctcggcccgaccctgcgcgaggaagaccagcccttgacctcggatcattactggctcgaggccattctggtgatcggttcgatcgtgaccttcttcacattgatcgtgatggcgttctccggatcgcttgccgggtattttccgttctcgtcgctggtcattccgctggcctggatggcggtcatgttcttcggatcgcgctgggttcgtgacaacaagccgtcgggattctttttctccgacctctggtacgaatttttcatggggctggttccgccctctgcgctggtcgccttcgcgctaggctcgatcctgttcggctgggccaccccgaccgagggtgccggttgcggggcgttcggcgcgctgctgctgaccattgcctatcgcaagctgtcggcgaagatgttctatgacgcgctaatcaagacccttgagatcaccgtcctgatcctgttcctcgtcgccgcgtccaacttcttcggcgctgtgttctcgcgccttggaacgccgatgctgctgaccgaattcctgctgtcgtgggatatttcggtcacctgggtcctgctgatcatcatggcgctgatcttcctgctcggctggccgctggagtgggtgccgatcgtgctgatcatcgtgccaatcctgatcccggtcctgatcaagctcgacgtcaaccttgtctggttcggcatccttgtcgccgtcaatctccagacggcatggctcagcccgccggtggcgctatccgcctacttcctgaaaggggtggtgcccgaatgggatctgaaggatatctatcttggcatgatgcagttcatggtcattcaggtaatcggcctggtcctgatcttcgtcttcccgcaaattgccctttggcttccaactttgatttacggagaataaatccgacatggccgtgacctatctgaaacgcgctgacaagacccctcagaccggaaccgacgaggcccgcgagattgtccaggcgatgcttacgaagatcgaggcaggaggcgaggacgccgccatcgcctatggccgcgagctcgatggatatcacggcgatatcgtcgtgtcggccgatgccatcgcggccgccggtgacgagatttcccagcggttgaaggacgatatccgttttgcctatgaccgggtgaccacctttgccgaagcgcagaaaaagtcgatcaccgaattcgagaccgagttgtcgccgggtctctgggccggtcagaagctgattccgatcgaaacggcaggctgctatgttccgggcgggcgctatgcgcatgtggcctcggccatcatgtcgatcgctacggcgcgggtggccggtgtcgagcatatcgtggcctgtaccgcaccgcgcggtgatgccgggccgaacccggccatcctgtatgcgatggaccagtgcggtgccgacaccatccttgcccttggcggggtgcagggcatcgcggcgatgaccttcggcctgttcaccggcaagccggcccgcatcctggtcggtccgggtaaccggttcgtcgccgaggccaagcgcatgctctatggccgcgtcggcatcgacatgttcgccggccccaccgaaatcgccgtgattgctgacgacagcgccgatccgcagattgtggccgaagatctggtcagccaggccgagcacgggcccgactcgcctgcatggctgatcaccacctcgcggcagctggccgatgaggtgatggcgcgcatggacggcctgatcgccgcactgccggaagtggcacgcggcgccgccaccacggcatggcgcgattacggtgaggtgatcttgtgtgacagcgacgaagaggccgtcgccgtttctgatgaatatgccgctgagcatctcgaggtgcataccgcccgtgatgactggtatgccgcgacattgcgaaattacggctcgttgttcatcggcgaggaaaccaccgtcacctacggtgacaaatgttcgggaaccaaccacattttgccgacaaagggcgcggcccactatacgggcgggctttcggtgcataaatatatgaagatcgtgaccacgcagcgcatgacccgcgaggccaaccgcgaggttggccaggcggcggcccgcatttcccgtctcgagggcatggaagcccatgcccgggcaggcgatgcgcggctgcgtaaatattttccgggcgagaacctggcctgaccggccggcagtctgccaattcgaagagagaggatagagaccggatgtcagtgaatgatgtcatcatggacgcgatgatcgagaacaatgtcggtttcgtgaccactgtgccctgcaagcagcttgccggggtgatcgaaaagatcgagcagtccgacaagatgatccatgtcccgtcgaaccgcgaggatgaggggatggggctgtgtgccggcgccttcatgggcggcctacggccggccatcataatgcagaacaccgcgatcggcgtgacaatcaattcgcttgccaccctgatacagtattaccgaattccgttgccgatgctgattagctatcgcggcgagatcggcgagccggtggcctgccaggtcgagatggccgtccataccaaggcgcttctcgatcagctctgcatcccgacctaccatttccacaaggaagaggatgccgacgaattgccggcgattctcaaccacgcctacatggcccgcaagccgatggccgtgctgatggatgccagcttctggaagggatattgagaccatgatccgcagcgacgttctgaaaaccctgatcccgctgatctctgacgagctggtggtaacgaatatcggcctgccaagccaggagatgcatcttctggacgaccagccgacgaacttctacatgcttggcacgatgggcctcgcctcttcgattggcctcggcctcgccatggcgcagccgaaaaaggtgatcgccatcgacggcgacggctcggtgctgatgaatcttggcacattgccgacgatcgccaacaaccaggtcccgaacttcatcctgctgatcatcgacaatggcagctacggctcgaccggcgatcagacgacctatgccggcatgcgcacctcgcttgccgatgtggcacgggcctgcggctgtgaccatgtggtggaatgcgccgccgaggatacggccgccgccgtcgaggccgcgcttgccgccgagcagacaacggtgatcgtctcgaaatgcaaatccggcaatatcaaggtgcctgtgatcgagcttgatcctgtgatgatcaaggaacggttcatggcatcggtcgcggaataaaggcgcgcgaggcatggtggaggtgcagcagaatcttttcagccttgatgggcatgtcgcgcttgtcaccggggcgagctcgggtcttggccgccgcgcagccactgtgctggcggatgcgggtgcatgtgtcatcggggtggcacgccgtgacgacgcgctgaaaagctgggttgccgagggtgccggaaaccgcgccgctcttcccgccgatctctcggacgaggccggagtcgccgccatgtgcgaggcggcatcggggatttttggagcacccgacattctggtccatgcggccggtgtcaatctgcgccagcctgccgaggatatcacgccgggtgactggcagctgacacaatggctcaatctcggcgcgcccttcttcatcagccaggcgttggttccggcaatgaaggaacgcggctggggtcgcatcgtcaatttcgcatctttgcagacgacgagggcctttccatcaggcattgcctacggggcgtcgaagggcggtgtcggccagatgacgcgggccatggccgaggcctggtccggcgacggcatcaacaccaacgccatcggcccaggctttttcccgaccgagctgacggcggcggtttttgccgacgaggcgcgcgctgcccgcaatgcggcgcagacatgtatcggccggaatggcaggctcgaggatatcgacgggccgttgctgtttctctgttccgatgccgcggtctatgtgaccgggcagatcctgatggttgacggggggttcacggcgaaatgaaggcgcttgtctataccggtacgaattcaagtgagctgcgtgatgtcgaggcgcctgtcgccgcgggcgggcagagcattgttgatttgtccttctgcggaatctgcgggtccgacatgcatgcctggcacggccatgacgagcgccggatcccgcctctcgttctggggcatgaggcagtcggcacggcgctgaccggaccgctcgccggacggcgggtggcggtcaatccgctcatgacctgcaacgactgccccgcctgcgacagcggcaaccatcatctttgcggcacgcgcgagctgatcggcatgcgttatcccggggcttttgccgaaaaggtaatggtgcccgacgccaacctgacagtacttgccgaccatatgtccttttccgaggcggcgctggccgaaccggcagcggtggcggtgcgtgctgtcgagattgctgccggtaccggcgcaacagcagattcccggatcgtcatccttggcggcggcgccatcggcgtgctcgtcgcgcaggtgcttgctgcgagaagcttcgcgcggccgcgcatcgccgagaccaacgctctccgccgcggcatgctggaaaatatcggcatcgccggaagctatgatccgcgcgaggagacaccggaagacggctcggtcgacctggtgatcgactgtgtcggcatgggcgccacccgcgccgcagcatcggcgatggcgcgcccgggcggtgtgattgtccatgtcggcctgctggacaacgagccgggcctcgacacccgccgcctgacgcttcaggagatcgggttcttcggagcctattgctaccggcatcaggatttcgcgactgcgctcgaccttttgaccggcggcaaggtgaccggagaaggctggaccgagatccgtccgctggacgatggcgcgcaggccttcatcgatattgaccagggccgcgctccgccaaagattatcctcgccaccggctgacgccgggactggacaatgagccctgccatgcccatctgctgggcatgaacgagacaattccccttccgaccagattcgccgaccgcgcggcgatgctggctcatgtggtggccctgtcgggccgtgacgatgccgcaccggcggttcccgatatcgccgcaggcggcagaaaggctgccgaggccgtgctcgtcagggtcgacccgagggcctatgctgccagccgcaaccatctcgatggcgcggtcacgcggctgtcaccctatatccgccacggcgttctgagcctcgacgaggtgcgtaacgcggcgctggcatccgttgttgatccgaaacaggcggagaaattcattcaggagctggcctggcgcgattactggcagcgtatctacctggagcatccggagcgcatctggaccgatgtcgaatcctacaagaccgggttcgacgccgccgaatatgatgccgagctgccggcggatatcgccgccggcgaaactggcatcgcctgcatcgaccagttcatcgcgacgcttgtcgagacgggctatcttcacaaccacgcccgcatgtatctggcgtcctatacggtgcactggcggcgggtgtcatggcaggccggcgcgcggttcttcctcgagcatctggtcgatgccgatccggccagcaacaacctgtcatggcagtggatcgccagcaccttctcgaacaagccctatatcttcaatctcgagaatgttgcgaaatattgcgggccggacatcaacaccatcccgcggcataacctggttctcgaccagtcctatgaacatctgagcgagctgctgttcccgtatatgggaggtccgcatggctgatcttgtctggcttcacgaaggggcattgcgtctgacgcacccggtgtttgtcgccggtggcccggatgccgccaccatcttcatctgggacagggaccgcctcgcggcgagccatgtcggcatcaagcgccagctcttcatctacgaaacccttgccgagatgtcccaagcccgcagcctcgacatccatgagggccgggcggaggaggtactgccgcgcctcgccgcgcaggcgggtgcttgccgcgtgctggtaccgtcatcgcccgacccggcgctgcgtgccgagatcggtgccatccgccgcgccgcgccggacctcgagattgtcaagatcgaggagacgccctttgtgactcttgcaaaaacgccggacctcgcccgtttcttccgctactggaacaaggccaggaagagcgcgctccgcccgcacggcatctaagccgccaacacgggaaatccattgatccgccgcgccggcatccggtatgttggcggcagttccgaagttttttccgagacgcccagatgaccgatattcttgccgaactcgaggccaaacgggcgcaagcccggcttggcggtggccagcgacggatcgatacccagcatggcaagggcaagctgacagcccgcgaacggatcacccttcttctcgacgatgcctcgttcgaggaatgggacatgttcgtcgagcatcgctgccatgatttcggcatggaagaccagaaagtcccgggcgacggcgtcgtcactggatacggcaccatcgccggacgtcccgtctttgtctattcgcaggatttcaccgttctcggcgggtcattgtcggaaacgcatgcggcaaaaatctgcaagatcatggaccaggcgatcaagactggaattcccctgatcgggttgaatgattccggcggcgcgcgcatccaggaaggcgtcgcctcgcttggcggctatgctgaggtgttccagcgcaacgtgctggcatcgggcgtggtgccgcagatctcgctgatcatgggcccttgcgccggcggcgccgtctattcgcccgccatcactgatttcatcttcatggttgaggggtcgagttacatgttcgtcaccgggccggatgtggtgaagacggtgacccacgaggacctcaccgccgaagagctgggcggggcgaagatgcacagccgcacatcgggtgtcgcccatggcgcgttcgagaatgatgtcgagatgctgatgcgcacccgcgcgctgatgtcctatctgccgctgtcgaaccgcgaaccggcgccggttgttccgtgccacgacccgatcgaccgtccgtcctcggtgctcgacgcgatcattccagataacgctaacatgccctatgacatgcgccgggtggtgctggagattgtcgacaatggcgagttcttcgaaatccacgagaatttcgcctcgaacatcattgtcggcttcggccggatcgacggctgcaccgtgggcattgtcggcaaccagccgatggttctggcaggctgtctcgacatcaacgcctcgcgcaaggcggcgcggttcgtgcgcttttgcgatgccttcaacattccgctggtaaccctcgtcgatgtgccgggcttcatgccggggctggcgcaggaaaccggcggcatcatctcgaatggcgcgaagctactgtttgcctatgccgaggcgacggtgccgaaggtgacgctaatcactcgcaaggcctatggcggtgcctatgacgtgatggcgtcgaagcatctccgtggcgatgtgaattacgcctggccgagcgccgagatcgcagtgatgggaccggaaggcgcggcgcggatcatcttccgcgaggatgccgccgatctggaaaagctggccgccaagaccgacgagtatcgcgagaaattcgccaacccgttcgttgccgccgggcgcggctacctcgacgacatcatcatgccgcgaaattcgcgcaggcggatctcgcgggcgctttcgatgctggccgacaagcagctcgagaacccgccgcgcaagcacgacaatctgccgctttgacccgaatgtgacccagcagcaccgcgatacagggaccgtttcatgtttaagaaaatcctgattgccaaccgtggcgaaatcgcctgccggatcatgcgcacagcgaggcgcatggggatcaccaccgtcgctgtctattccgacgccgacgctggcaccccgcatgtgatgatggccgacgaggccgttcacatcggcggatcggcttcggccgacagctatcttcgcggtgagcgcatcattttggcggcgaaggaaacgggtgccgaggcgatccatcccggctttggtttcctgtccgagaatgcgggctttgtcgccgaagtcgaggcggcgggccttgtcttcattggtccgggcaccaaggccatcgcggtgatgggcgacaagatcgaatccaaggcgctggccaaatccgccggtgtctcgaccgtgcccggaaccgagggtgaggttgatgacgtcgaggtcgggctgaaggccgcggccgagatcggttatccagtcatggtcaaggcgtcggcaggcggcggtggcaagggcatgcgcgtcattgaaagcgccgacgatctttccgacgggatgcgggccgccatgtccgaggcgcagaccgcctttggcgacagccgcgtcttcatcgagaaattcgtggttcagccgcgccatatcgaaatycagcttctggccgaccagcacggcaacatcgtctatgtcggcgagcgckaatgctcgatccagcgccgccaccagaaggtggtcgagnnnnngccgagcccctttatctcgccggagacgcgcgccgccatgggtgagcaggcggtcgagctggcgcgcgccgtcggttaccgctctgccggcacggtcgaattcatcgtcggcgccgaccaggatttctatttcctcgagatgaacacccgccttcaggttgaacatccggtgaccgagatggtctacgggctcgaccttgtcgaaaagatgatccgcatcgcanccggtgaagagctgggtctgacgcaggacgacatcaaggccgacggctgggccgtcgaggcgcgagtctatgccgaggacccggcgcgcggctttcttccctccattggccagctggtgcgctatcncgagcctgcaggcgagggcgtgcgcgtggattccggtgtcgatgagggcggcgagatctcgatgtattacgatccgatgattgccaagctggtcgcccacgcgcccacccgcgacgctgccatcgacaggctgcatctggcgctcgaccattacgagatcgacggcatcgtcagcaaccgccagttcctgtcggcggttctggaaaacgggtccttccgcagcggcgacctgacgaccggcttcatcgatgccgaatttgagggcgactttgtggccgggccaccgtccgatgctgccacggcaaggcttgtcgccctcggcaccgccatcgtcgcgctgcagatggaggaacggcatgctggcgacccggataccggctttgtcgtgatcagccaggacggcacccgccgcatggtcgagcttggccatgtcgccaatcgcagcgccaagctcacagtcgacggcaaggggttcacacttctcggcctgcttcaggcaccggtctatctgttcgacggcacgatcaacgaggaacccgtcgctgtccagatccgccattcgggtcacaaggtggtgctgacacatggcgcagcacggcttgcgctgacggtgcttccggcgcggttcgagccgatgctggatcatatgccggtagccgccgagggcgctggtgccgacgagatcaccgcgccgatgccgggccagatcacccgtatccttgtcgaggtcggggacgaggttgtcgaaggtcaggacgtcgctatcatcgaggcgatgaagatggagaatgtcctgtcggccgaagcgcgcggtacggttcgcgaggtcgccgtcaagattggcgacaatctgaatgtcgatgatttgatcctggcgcttgatctcgccgaggacggctagtccgataccagcatctgttcggcaagcgtggcgatccaggccgcgccgttgccgaggatttcgtcgttgaaatcatagttgctggcgtgaaggggccgtgctgacgcgccttctgtgccgttgccgatcagcatgaagcatgcgggcaccacgcgggccatatgggcgaaatcctcggaaaacagtttcggatcacaggccgggttcacccggtcctcgcctgcgacggcgcgggctgccgccacggccgcgcctgccgcctcgtgatgattggcgagggcgggaaacaccgtatcgtaagcgacctcgatctccacattgtgggttgatgcgatgccggcacagatccggcgcatggcagcctcgatagcggtgttggtatcatcgccgagtgccctggcatcccccttcaggacagcggttccggggaggacattgcggcggccgtcggtgataaattcggtgaccgagacgacaccgttgcgcgccgggtcaagtgtgcgtgagacgataccctgaagtgcggtgacaagctgcgcgccgacatagatggcatccgcgcccatatgcggcatcgcggcatggccgccgcgtgcactgatggtgatctcgaacaggctctcgctggcggtcatggcgccggcccttgtggcgaaacttccggccggaatgccgggaatgttgtgcatcgcgaagacggcatccgcctcgaagcgggtgaacagcccgtcatcaatcatcgccgtggcgccaagcccgtgttcttcattgggctggaaaatgaagattgcgcgtccgttgaaatcaccatgctgtgccaggtgcgccgcggcgccgagcagcatgctgctgtgcccgtcatggccgcaggcatgcatcacaccgtcatgtgcggaccgccattcgggattgcctgtctcggtgatcggcagcgcgtccatatccgcccggaaggcgacgctggcctcgccgttgccacgctgcaagacgccgacgacaccggtgcggccgacacccgtatggacctccagcccgaattcccgaagcagttcggcaacacgcgctgatgtgcggtgctcgttaaagcccaattccggatgccggtggaaatcgcggcgccagccggtcatgtcggcaacaagttccggggcgatgttcgatgtggtcatgcggcgtcctcgcggatcatgtctgacgccttttcggcgatcatgatggtcggggcgttggtgttgccggacacaagggccggcatgatcgaggcatcgacaacgcgtaacctgtcaatgccgtggacccgcagacgcggatcgaccacgtcgccctgctgcgctgacggccccatacggcaggtcgagacagggtgatagagcgtcactccggtggcccttgcatgcgccagcagcgcgtcgtcgtcatcgatatccgggccgggctgcatctcgtgcatcacatgcggtgccattacatcgctctgcatgatatcgcgggcgatcttcatggcggcgacatggacccggcaatcctcgtccgcagtcagatagttcggctgaatcagcggggccttcatcggatcgggactggcgatatggatcgacccgcggctttcgggtcgcaacatgcagggtccgaaggtgatgccggggaaagtgtcgaactggcgcttttccggatttgcgaagctggcattggcgatgtgatactggatgtccggtccggcaaggccctcgcggcttcgcacaaagccgctcagcatgccggccggcatcgacagcacgccgcgccgggtcagaagatagcgcatcacctcgctgaccagaccgaacccgtgggccagcttgttgatcgagatgtcgctcttcagccgccatgacagccgcgagatataatggtcgtggaaattctcgccgacaccggcaagcgcatgccgcacagcgatgcctttcgatgccagcaggtacggatcgccgatccccgatagctcgaggatctgcggtgactggatagcgccggccgacagaatgacctcgcgcccggcgtggattgcctgttcgcttccacgtctgcggaaggtaacaccggtggcgcgctgcgtgccgccggcctcgccctcgagggtgaggccggtgacatgggcttgggtgatgacacgcaggttcggccgcatgcgggcatcttccagataggccttcttggccgagaagcgcatgccgtttttctgcgttacctgataatagccgaagccgtcctggctggcgccgttatagtcgggattgtgggggtagccgcagctctttgccgcctcgatcagcaggtccagcgcctcgtacccgttgcgcaacccgctgacattgagcgggccacccttggcgtggaactcgtcgtcgtcacgggagaattcgcaatgttcggcgcggcggaaatagggcaaaacatcgcggaagctccagcccagattgccacattgtgcccagccgtcataatctgccgcctggccgcgtacatagagcatggcgttgatcgaggatgatccgccgagcaccttgccgcgcggttgtttgatccggcggttgttactggcttcgtgcggttcggtctcgaacatccagttcatcgccgggttgaccattgtcttgatatagccggcgggaacatgaatgaggggattgcggtcctcgccgccggcttcgagcagtatgacggaaaccgcggggtcctcgctgaggcgcgccgccagcacacatccggccgagccaccgccgataatgatgtaatcagctttcataacccgctgactttgacgtgttcgccgacggcgggcaacagcaaaacaggggggttccggcagcctcggtttgggcgtaggctctggccgatattaccatccggacgtcgtgccttttccatggacatgcttcttgtcttcctcctcgccgcgggccttagcggcggtttcgtgaacggcctttccgggttcgggaccgcactgttctcgcttggctggctgctgcaggtcatgcccccgcaacaggcggtggccatcgcccttgtctgcagccttgtcaccggtgtgccgggtgtctggcaggtgcgccacagcattgacaggcgcaggctggctctgttcatcctgccggcatttgccggcattccgatcgggacggtgcttcttggcttggtcgacacgcggctgctgtcgcttctcgtcggcggcatgctgctgctttatggcggctatttcgcctttcgccgcaccctgcccaccatcgaggggaactggaccctcgttgaagcggggatcgggtttgtcggcggcgtgcttggcgctatggcggggttgtcgggcgcgctgccgtcgatgtggctggcgcttcgtccctggccgaaggcggtgcagcgcggcatcatgcagcccttcagcatggtgatccttctggtggccacggtgttgcttgctcttgaaggggtgttcatgccgcaggtgctctacaatcttgcgctggtccttccagcgagcatgttcggcgcggtcatcggcctgatgctgttcaggcggatgccggacgccctttatccacgtgtgctgatcggccttatgctgatctcgggcggcacattgctggcgcgtacattgctggccggcggggcataggaggcgggatggcggacgatctgatctcgccggacgggttgccacgctgttttggcaaccgtcttggccaggatatcctcgcccattaccacgacaccgagtggggcgtgccggttcatgacgacaggcatctgttcgagatgctcacgcttgagggcgcgcaggccgggctcagctgggacgtggtgctcaggaagcgtgccagctacaagatggcttttcatgactttgacctctgccgggtcgccgccatgactgatgcagacctcgaagcgcttcgcgaggatgcgggggtcgttcgcaacaggctgaaaatctacagcacccgcggcaatgcccgcgccattctcgaccttcaggacgagcagggcagtttcgattcctatctctggggatttgtagatggccaaccgcaggtcaaccacttcgccgaccttggtgaaatgcctgcctctaccccactcagcgacagcatctcgaaagacatgaaacgccgggggttccgctttgtcggttccaccatcatctacgccttcatgcaggggatcggcatggtcgacgaccatgtcgcgggatgctggcggcgtagcggggcccatgccatgtgtccaatggagcggtgaacaatgtggtgagtggctagttgaggagtggtctgaacagcttcaaaatgaacatcagcgtgaaatatccaccatttatgccaagcacgaatgacaccatcatggttatgctgaatggcaggtcgtcattgaacatatttcgaaagaaattgcgcccgaggacaatcgaaagttctagaaacatcacagtaatcagcgctgcgttgaacaacataaaagttattgcgtcgcgcgacagaatcgcgacaccgatcaaaactgaggcgctgccatagataccgcccactggaaacagctggtaaaactgcctgcaccatcctttaggcaggaaccaaggggtggaaccatatgagcagtgggcatagcctaccgccgtgattagaaaggcatgtatcacccccacagtcatgagatgtgggctgtatgatgcgagaagggtttccatcgataatgtctagctttccagaaagagataggtttgagtgatttgtaaagtttttccctgttatacctaatcttaaagagggccaaaaatgttcaatatcaaattcaccaatcaatggggtcttgttctcctgatagccgttggggctgcggtcatccatctgcagccagccggtctcgaggccgacgaacctgtccatcgtgagatgatcgctaccacagacaaagccttgtggcatcttaaggatggaaagcttcgctattgtatcatccgatcctcggaaatcttttgtatcgaggagacaaaatgataccgcgctatcgacggagcctttggttttaaacgactccgtaggttctatcacgcagaaactgggtgacagcctttcggttaccaagtgcactcaattcttcggcatggacagggcgccgcatcgtgagcaacaccgtacttcccatcagcttgcagatctcgcggaacatcagcgagtaggcaagcgtcaggctgatcttgcgggcagcctggaacagcagcgagttctgcccgtcgaacaggaacggcactgtgaccgtgtccggctgtatggcgagtttcgccgcgaaggtcttccattccttgtcataggcatcgccgaaaaggttcggcgcgagcgagatagcgcccgccgggaaaatgatcacagcaccgccccgcttcagatattcggcagccttctggcgggtatcgatatttgtggtgagtgccgcctcagtctcgtcgaaatcgatcggcaggatcttgtccatcgtcgcgggtgcctgttttagcacacggtgcgtgatgatcttgtagtcggaccggacttcggccatcagcgcgcagagcacaagaccgtcgatgacaccatagggatggttggcgacgacaagggtggggccggtgcggggaatcattgcgccagcgtcgcgctgcaggttgatcttgatgctcagcttgtccagcgcatccgcccagaaactggcatagggccggtcttcctcgacatagtcgaaatagagcctgcgaatcttcggctggccgctgaaccgctcgatggacctgatcagcgtattggaaatagggcccatttcgccgtcggcgaaggtgaatatcggatgtgcggtttgcgagctctgcattcctgccagcctttgttctgtcccgagccgtccaaactaacgtcgatctaagacagatacattacataaccgcaggaagtaggcgatttcggggcagactgtcaatgatcctcgggcagcccgcccgccgatgccgggagccatccccaccgcacggaacgccgccgccacacgactgcttccagttcaggcatacatgacctcttcaaccattgccctggtgggccggaaaccggggtttgcggatgccgacgcgaggcaaaacacaggttccggccggaatcgcgactttttcatttatattttataatatattgaaaaaaaatgataaaaatcgaattttgcccaaatcggcatctgaatcttgctgaattgataaaactgtttttcacgccataaccaattgttaataagcaataaaaacttggtgttgttcatgcggctgcaaacggggtccttagcaattgcgccgggcctgtttgtctcaatatggttacagcgtgacgggtgggtcattatgtcgctaaaggcatggcggctcgtccgtaccttttcgttaacggaggtaaaacaatgaaactctcaatgggtaaggtgatgaagactgcgatgatcgcaggcggactcgccactctcccaactgtcgcaaacgctgctacggcgatgcttgctgcaactgactatgtaggcatctcgttctggaccatttcgatggccatgatggcagccgcaatcttcttcctgatggaagcaggtcgcctgcctggcaagtggaagaccgcaatgacgattggtgcactcgtgcagatcgttgcagcggtacactatttctacatgcgcgacgtatgggtcagcactggtgaaacgccaaccgtttacaggtatgttgactggttgatcactgttccgctccagatggtagaattctacttcatcctggcagcggtggccgttgttagcgctggtatgttctggcgcttgatgatcggttcgctggtaatgctggttgctggttaccttggtgaagcaggccttgtgaatgcatggctcggttttgtcgtaggtatggccggatgggcatacatcctctacgagatctttgctggtgaagctggcaaggtgtccgcagagtccgcgccggaggctgttcagaagtcgttcagcacgatgcgtatgatcgtgaccgtcggctgggcaatttatccgctcggctacttcctcggttacctgaacggtgctgctgacgctgtcaccttgaacgttatctacaacgtcgctgacgttgtaaacaagatcgcgttcgttggtgtgatctgggcagctgccaacgcagaagccgcgaaagcctaaatgcattgatgaagggcggcacccacacgtcgcccttcatcacatttccttaatgacattttgtgaatcatcttgttgcgcgggaaggtgcggatattatgactgacctgtctacaccctcgaaacatgcatttgtcgagcagggttccgggctttccatttcgaagtccacttccgagattattcacgatttcatccttgaaagaatgcctgcccccgggcagacgctcgccgacgcggctatgcatcatttcgctgccccgggcaagatgctccgcgcaaagatggcgctccgtgctgccgaccttctcaaggtcgaccggccggcggcccttcactgggcggcggcgatcgaggttctccacaacgcgtcacttgtccatgatgacatttgcgacggcgaccagttgcgccgcggacgtccggccgtctggtcgaaattcggacgcgatgtcgcgctcaccctcggcgactggcttattgccctatctttcgagctcgccgcagaggccgcgcaacgcgcgcagacgccgatgctgctgaagattcttgccacccatatgaagacaaccactgtcggcgaggcgcacgaattcgacccggcgccggttaccgactgggatcattacctgcagggtgccggtgacaagaccgcgccgctgctgaccgcgcctctcgaggggatcgccgccatggccctccatggtggcgccgctccgacgatcggtgcctgcttccgctcgcttggcaatgcctatcagatcggcaacgacatcctgaatttctcgggaagcgacggtgccaatacctgcgggtcggacatggctcgccgtgccccgaacggcgttgtcgtccttttccgccagacactggatgcagaccgtcttgccgcattcgacagctggtatgccagcggctcggatgacggattcgatgcgtggctcgcagagctgcggcgctcgcccgccctgttcgaggcggcgcggcggatgaatgcgatgctcgacgacgcgacgggtcttgccgacacgctgccgcccgaaatggcagcggtgatcacgcctatacagcagcttctgcagcaggtatgtctgaagtctgtggcttcgatcaaacaggcctgacgcaagcattatggcaccagccccctcggctattcaggatctggcggccccgcattccaagaccgggccagccgaccagagcgatgttcttgtaatcggtgccggttttggcggaatcgcggctgccctccgtatgcgcgcccggggctattccgtgacgcttgtcgacaggctggatgccattggtggacgggcgcaggtgtttgaacggggcggcttccggcatgatgccggcccgaccgtgatcacagctcccttcctgttcgacgagctgttcgagctgttcagagagcgccgcgagaatcatctcgacttccggccgctggacccctggtaccggttccatttccatgacggcaaccagttcgactaccgcgccacggtcgaggacaccaacgccgagatcgagcgtttctccccgcgtgacgtcgccggatatgcagggctgctggaggcatcccggcagattttcgaggtcgggttcgagaaactcgccgacaggccctttactcgtttcatgacgatgatggcgcaagtgccctcgctgttgcgccttcgcagctatcacaccgtcgccagcctggtgaaccgccatatcaaacatccactgcttcgccaggccttttcgattcatccgctgcttgtcggcggcaacccgttcacgaccacctcgatctacacgctgatccattatctggagcggcggtggggtgtgttcttctgtatgggcggcaccggcaagctcgtcgaggaactgcaccggcttcttgaacgcgccggcgtcaatgtcgagcttggcgtggatattgagcgcatcaccatgaaaggcggccgcgccaccggcgccgaatccacagctggcaggcgctatattgcggatcgggtgatctgcaatggcgatccgccgaccgtctatcagcagatgctgcccaacgagacacgctccaagcgggcaattcccgaagccgcgacaaaatattcgatgggtctttacgtgatgttctttgggacacgcaagcagtatcccgacgttgcgcatcataccatctggatgggaccgcgctatcgtgagctgcttgccgatatcttcgaccgcaagattcttgccgatgatttctcgctctatcttcaccggccgaccgccaccgacagcagcttcgcgccggacggctgcgacagtttctatgtgctgtgcccggtgccgaacctgcttggcgatgtggactgggagtttgagggaccaaggcttcgcgaccgcattgttgcggcgctggaagagacgatcatgccgggcctgggcgcgtccattaccgatgatttctggatgacgccggaagatttcaagcgcgactaccgttcgatgcatggcgccggtttctcgatcgcgccgattttcacgcaatcggcgtggttccgctaccacaaccgcgacccacatatcccgaacctgtatttttcagccgccggcgcgcacccgggagcagggatgccaggcgtcttgtgttctgctaaggttgttgagcgactgattgaggaggaagaccgggcggccgccgccagcggaaggctggcaaagggggcctgacggcgggagccggcactcatgtcgaacgaagagctgacatccgaaaaatcgcttgccgccaacggcaagagtttccactgggcacgccgttttctgggacagcgcatgggaagcgacgcggcctcactctatgcgttctgccggcttctcgacgatatggccgacggcgatatacagaacggtccgaccaggcttgccgccatccgtgccgatctgattgccggccgcgagggggctgatcccgcattccttgcattcctgccgctgatgcgggaaaaacgctttccaacggacgtgcttgtggcgctgatcgacgggttgctcgaggatcaggccgaagaggtggcgctcgccgacgaggccgcgttgctgcgctacgcctaccgggtggcgggaaccgtcggcctgctgatgtgccatgttctcgactgccaagcgccggcagcacgggcgcatgccatcgatctcggcatcgccatgcagctgacaaacatcgcccgcgacattctcgaggatgccggcatgaaccggcgctatgttccaggtgactgggtcggccatgcaagcccgcacgaaatccgtgccgcggcaaggtcgccggaaagcatgctggcgcgcgatgtgaccgctgcggcaggccggcttctggatcttgccgagaccttttatgccagcggggcccgggggtatcgctatctgccatggcgggcgcatctcgccatcggggtggcggcacgggtctatcggcagatcggcatccagcttcgcggtgccaaccttgcctggcatgccggaaggcaggtgaccggcaaggcggccaagctgcgatgctcgctgctggcgctgggaagttttgggcgtcggatcggcatcgggacatcgccacatgacgaccgccttcacgcagcactgcgggggctgccctatgtcgcctgaccacgccatgcggcaggttcgtgtccttggtgacggctgcgccgcgctgtcgctggcggcgcgcgccaatgaactggcgggccataggctgacacttgtcaggccgcgaggcgccccgcaggagacggaccatatctggggtttctgggacggaccgaaccttgccgccgccagccgccttgcccgcaagcgctggcacagctggtcgatcgtgacccgccggggaaaggctgtattgcgatcacatgaacggccctacacggcgcttcaccggctggactggacggcgcattgccgtgaagcggccagacgtgccggcgttgcaatcctgaccggcggggaagctgcgacagcggcagaccaggtcttcgacagccgcccgccgccagtgccggacgacataatgctgcagcattttcttggcctcgaagtgcgaagcaagcgtgcgattttcgatccggataccgccattttgatggacttccgtgtggaccagtcgcgcggcatgcatttcatctatcttcttcccttttccgcgaccgaggcgctggttgaatccaccctgttcacgccatctgtctgtccgaagagctattatcgcagggaaatcgtcgcctatctcgaggcccatttcggactcgatgaattcgagacgcttcgcaccgaacgcggggtaatcccgcttggcatcttgccgccgcgcgatccgtccattccgggtgtaggcggcaatggtggcgcgatccgcccgtcgagcggctatgcctttccctttatccagaaacagatcgaccgtgccatcgccgcggcatgtgaggggcgcgagctcggctttgctatgccgcaccgccgtgttgatttgtggatggacgcggtgctgctgaccgttctgcgccactggccggaacaggctcccgaactgttcctgcggatggggcgtgcgctcgacggtgacgagttcgcacggttcctcagcggcgaggcggacttgccgctccggctgaaagtgatcatggcgatgccgaaggccccgttcctgcgcgggcttgtccgccttctggccggcagccggccacaacgcacggcagaggcgggatgatggaaatgatgacgtccatagcggcgatggcgccgctcgaccttgcggcgctggccgccatagtgctgatcggcctgccgcatggcgcgctcgacggggcgatcgccatccatctcggcttcacccggaaaattctgcatttcatttggtttctggcgctttatatcgcgatggccggggttgtcgtcgcggcatggctgctggcgccgaccgcctgcctgctgggcttcctgattatcagcatgctgcattttggtgctggtgacgcgcgccacggaaccggctggctccgcagtgccgagattctggcccatggcggccttgtcatcgtcggcatcagccagatgcatcatgtagaggtagatgtcatctttggttatctgaccgggcgcgatacggcgctcctctggcaaggcatcgatgttctgacgttgattgtcgggatggctgttgtcgtctgccttgcccaggcgctctggcatcggcgctggcgcgggacggctttcgagctcggcgcgcttgcggtactgtttgcgttgacaccgccattggtcggttttgccgtctatttctgctgtgtgcactcggcaaggcatgtctacggcatcgttaacagcttgcggcgcgaaatctcgcggttcagcctgctcaaccaggccgctgccttttccgtggcaagttgggttgccggcggtgccgccatcttgtggtttgccgatatgtccaatcctgagcctttcgtgctacgggtggtgtttatcggccttgcggcgctgacggtgccgcatatgattcttgtcgatggtttcttcaggcggtccggcaggaccctgaagcgccagtttatcagccggtaggtgcctatggatgatatgcatgaaaacgctggaacaccgtccgcaggcgatcgcaaggatgcgcatcttgctctggccgccgacgcattgtcccgctccgggattagtgccggcttcgagcgtgtgcggctcgagcattgcgcccttcccgagtgcagccttgccgatatcgatatcaccacttcctgccttggcaggcccgtcgaggcaccgctgttcatcgggtcgatgacgggcggcacggcgcatgccgatgcgatcaatgcggtgcttgccgacacagccgaggccaccgggatcgcgctggctgtgggctcgcagcgcgccagcattgaatcaggccgttcccaggcggtactgcggcagcgcgcgccatcggtgccgctgatcggcaatctgggcggtgtgcagctcgccgcgccgggtggtattgatctggcatgccgcgccgtggtggatatcggcgccgatgccatcttcatccatctgaacccgcttcaggaggcggtccagcccgagggcgagacggactggcgtggtgttctggacgcaatcgagacccttgtcggtgtgctggaggtgccggtgatggtcaaggaggtcggcgccgggatcgggcccgatgttgcgcagcgcctgtttgatgccggggtccatgctgttgatatcgccgggctgggcggcacgaactggacacgtatcgaggcggcgcgccgcgaggatgccgccctgttcgagccgttcctcgactgggggttgccgacggtagatgcgctccgcgctgtcaggagcgcctgcccgaacgccaggctgatcgcatccggtggtgtggaaaacggtctcgacgccgccaaggcattgtggcttggcgcggcgctggtctcgatggccggaccagtgctgcgcgtgctgactggcgacgggcgtggtgcacctgacggggccgcggcggtgcatgtcatcgaacgctggaaatcgcagcttcgcctggcgctgttcctgaccggtgcgcccgacctgccgtcatttgctggcaagcccggtcatgtcggtgggccatatcagagggccattggtgcgcagaaccggtgaccctgtctagaaattgtccttgcggcgcctgacctcggcgaatacctcggcatcgctcgccccttccatgccaagatgcgcacgcaaacccgcatcgctggcgcgaaggaatgggttggtggccttctcgagcgcgatcatggtcggcacggctggctggccgatggaccgaagctcgtccaccgcggcggcgcggcgccgcagcgcggtgtttgccgggtcgacgctcagcgcgaacctggcgttggcggcggtatattcatggccgcaatagataaccgtgtcgtcggcaagcgccatcagcttgcacaggctgtcccacatcatcggcgcgtcgccctcgaacagccgtccgcatcccatggcaaatagcgtatcgccggtgaacaccagtttttcggccggaaggtagtagttcagcatggtcagcgtatggcccggggtcgagatcacctcaacctcgcggccggcaaaggcaaagctgttcccgccatcaagcaccgtgtcgatcccgccgatctccgccgggccaaagacctcgacaccgtccccggcgagcgcgccaaggcctgccgtatggtcgccatgatgatgcgtgatccaaatctgggtcagcggccatccggcctcgtccagcgcggcttccgcggcttcggcgtcaccggcatcgaccagcgcggtctcgccgcgttccgcgtcgtgaagaagcacgccgtaattgtcgtcattataggggaactgatggatggccagccgcatcgctctaccagaccccggtgttttccatcgaggcccagggctcggccggggcctgcgcctcgccctcctgcagaagctcgatcgagatgttgtcgggcgaacggacaaaggccatgcgcccgtcgcgcggcgggcggttgatggtgatccccgcatccatcagcctctggcaggcggtgtagatatcgtcaacgtaataggcgagatggccgaaattccgaccgccgtcatattcttccggatcccagttgtaggtcagttccagctcaggagaacgggttgccttggcgctatcgagattgccgggcgccgccagaaagacgagcgtgaaacggcccgcctcattctccttgcggttggtttctacgagcccgagcttgttgcagtagaaatcgatcgacgcgtcgaggtcagtgaccctgaccatggtgtgaagatagcgcatggagcccccggtaaatggcgcaaaaaaggagcgcgtagcataggatattgcagggcggcacgtccagttgtgccaacaggcatgcggctggatatccgttcagatatcagggcatcagccgcgggaatagaggcccagagtttccgcctgcaatttcagcagcgcggtgacatgcgccagtgccggcgtctcgatgcccagtctttcggcaagttctagaacggcggtgaccagcgggtcgatctcgagcggtcgtccggcctcgatatcctgacgggtcgagggcttgtgcccgacaatcgcggcagcgccgtcgatgcgcttttcgatgggaacgctgaaccgcacgccgatggcctctccgacactcttcacctcggtcatgatggcatggacgagggcgcggctttccggatcattgccgatggcatccagtgtcgcgccggtcagcgccgagaccgggttgaatgagcagttgccccacagcttgacccagatttcgtcacggattcgcggcttctgcggcgccttcaggccgccggctatcatcagcgatgacagcgtcttgacgcgctcactctgctccatcgacggctcgccgagtgtaaagcggtcaccgtcgagatgttttacaaggcccggcgcggctatctcgcaggccggatagacaacacatccgatggcgcgctgcggtccgatcttgttccagacgcgcccgcccggatccaccgcttgaagcggcttctcgtcgagtgctgttccagtctgcgccttgtggaaataccaccagggcaggccgttgacggcaggcacgacagttgtgtcgccgcccagcagcggcatcaggtcgtcgagtatcatcgggatggtatgcgccttgatggcgaggatcacataatcctgcgggccgagatcggccggcctatcgcttgccggaaggctgtagcgagttaccttgccgtccttctcgagtcccagtccgtccgccctgatggcttccagatgtggtccgcgggcgacaaggcttaccttggcgcctgcctcgtgcagggctgcgccgacatagccgccgatggcgccggcgccgaatatacagatatgtgtcatcgctttgctcccttgggcctttggctgaggcctttgactagtcttcgaggcgcagcttctgggcaaggccgatccgctgcagcttgccggtggcgcctttcgggatttcgtcgaggaacacgatgtgcttcggaatcttgaatttcgccagatgcgtttcggcataggcccgaagcccggcggcgtccagttcgccgccatcggcaagcaccacggcagcgccgatctcctcgccaagcatacggtcggcgacggcaaaggtgaccacctgctggacggcaggatgttccatcagcacgttatcgacctcgagaggcgagaccttctcgccgccgcggttgatgatttccttgaggcgtccggtgattttcagatagtcgtcaccatcgaagaatccctggtcgccggtacggaaccagccactggtgaaacttgattcattggctgccggattgttctcgtatccgggcgtgacattgtccccgcggatacagacttcgccttcggcgtcaccggtcagccggttgccttcctggtccatgatgcagacttcgggcgaggtggcgatgccgacaaagccggccttctgcttgccgttgcccagcgggttggatgtcatctggtgggccgcctcggtcataccgtaggcttcgatcaccggacattcgaacacgtcgttcagctcttcgaatacggccggcggaagcgacgccgacgaggagcggatcaggcgcagacccagacccttggcagcatccgcctggcgcttggcgcggagcaggatggcctggtgcatggttggcacgccggaataccagctgatcttttcgtcgcgcgcctggtcgaggaatttcagggcgttgaatccgccggtgcagcagaccgaagcgcccttcgacatgctggtggcgagaaccgcgatcagcccgtggatatggaacagcggcatgatgttcaggcagtggtcgctgtctgtcagctcgaggctggcggtgatattgcgtgccgatgccatgatattgcgctgcatcagcggcacaaccttgggccgcgatgtggtgccggatgtgtgaaggacgagcgcctcgttgtcggcgccggccggagtcgcattggcctctgcctcgaacagcctgaaagcgccggccgggtcgttgtcgccgaccactgcctcgaccaccgggatcgacagcgcgctggccgcggcacgcacggggttctcgctctccgcctcgacgatgaccagcttcggcgcaaggtcctcaaggtagaaggcgtattcactttccttataggcggggttgagcggggcagccgacatatatgccgcgactgccataaaggctgaagccatttcaggaccgttcggcagcacgatggcaacacggtccgacggtccgagcccgttcccggcaagctggcgcccgaccgaatcgacgtgtcggcggagggccgcataggtcatggcagggcgatcgggtgcggtgagagcgacggcattgtcggcgccggaatcaagcaaagtgcggatggtcatgttcggactcgtctttcctgaggggtatgggatcggacgtagatattcggacctgctttcgcaaggcctcgcaacgaatccggaatagctagcacaaagccgacagcatttgtaattcaattcgaggtaatttaggtaaaaagtaaaagtaaaaattttacagttttctatttttctcatttttccttgttctgagcgcagatttcgtgaaacctccttccaacgcgcagccggcatcgagcaacgggcggctgctacccatcctggctagttcgatccatgtcggttctgacgccgggacccccgagtgccaaacacagacgcggccgagggtcgcaaaggcaacgatggcggatcatcttgatatccgccatcccaaagtgcagaggagagagacatgaaagcacttcgtaaactcatgacatcagccttggttcttgggtgcgcaaccgtggtggtggcgagcccggcgatcgcatggcaacccaacaagccgatcgatttcatcatcatggctggcaagggcggcggcgccgacaagatggcgcgcctgatgcagggcatcgtggaaaaggaatcgcttgccgagcgcccgctggttccggtgaacaagtccggcggctccggcgcagaggccctgatggccgcgaaaagcgccagcgatcccgaccacaccatcatggtgacgctgaattcgttctacacaacgccgcttcgccagcctggacttgaggtcgacgttctcacattcgcgcctgttgcccgcatggccgaagacactttccttctctgggtccatgccgataccggcatcaccacattcgagcagtggcttgacgtggcgcgcgagcagggcagcaagtgggtcatgggcggcaccggctccaactctgaagacaacattatcaccgacttcctgaacaccaattatggcctgtcgatgaaatacattccctacaagggcggcggggccgtggccaaggacgttgccggcaagcaaatcaactcgtctgtgaacaacccgtccgaagccatcggcttctggcagtccggcgacatggttccgctggtcgccttcaccaacgagcgcttgccgatgtttccggaagttccgacgctccgcgagaagggcggtgagttcagctacttcatgcagcgctccgttgtcggtgcacccggcatgtccgaagaagctcgcgcctactacaccgagctgttcacaaaggtctacaactccgaagactggcaggcttacaagtcgaagaagtcgctaatgggcgacctcatgagcggcgaccagttgtctgactactggcgtgaccagcgtgacaatcacgaacagatcctccgctcgtcaggtgccatccggtaacccccaacgagaccaactggccgggctccgctttgtcggagcccggtatatttttcagcatttgcctttttattcgaagaccccgttgagtcgggactgggaccaggagcatgcgccgcgcggaaatcatcacagctttcattctcgggctgttttccgtctacctgatgtggaagagcggagagccgcctgcctggaacccggacgccgcgcgtttcgacaatatcggttatgacgacaccggcgcgccgggaagcggcttctggccgttctggcttggtgtcatcatgctggcaagctgtgtttggacaggcatcaactgggcccttcgccgtacgccggcctcggtctccaccgcgccgttcctcgactcatatggctggaaaatgctgctgactgtcggcggcgggctgatcgggttcctgctggtgatcgagttccttggcttctacgccgggatgctggccttccttgtctattacatccggttcatcgggcggcattgctgggcggtgacgctgtccatcgccaccgccgtgccagtggtctgcttcttcttcttcgacatcgccatgcggatcgttttgccgaaaggctatagcgagccgctgttcatcccgctctacgacatcttcctctagatatccagactttcccccgagacctgtatcatggacattctattcgctcttttcgacggtatcctgatcgccctgcagccgatgaatctcgggctggccatccttggtgtcgtgatcggcctgtttgtcggggcgatgccgggacttggttcggtgaacggcgtggccatcctgctgccgtttaccttcgtgatccaggggatcagcggtgacgcgacctcgccgatgatcttccttgccgcggtctattacggcgccatgtatggcggggcgatctcgtcggtggcgcttggcattcccggtgcatcgacggcggtggcgacaacttttgacggcaggccgctggcactgcagggcaaggcgcatatcgcgcttatggcggcggcgctggcctcatttgccggggcgactgtgtccaacatgatgttcaccggtttcgcgccgatgctggcctcggtggcgctcgatttcggcaatccggaaatattcgccctgatgctgctggccttcgcgacctttgccgggcttggcggggatgatttcccgaagacaatcttttccatctgtttcggcctggcgctgtccgctgtcggcttcgacatcatctccggcgagccgcggctgatcttcttcgatattcaggggttctcgcacggcatccgttttcttgttctggctatcggtatttacggcatttcggaaatgctgtggacaatgaacgcctcgcgcggcaagtcgtcgatgtccaaggtcgaggtcagtttcgccagcatgacaaacgcgctgatccggttcggtgaggcatggcgcggcacgctcatcggctcagttatcggtttctttgtgggcatcctaccggcggcaggcgcgacccccgcctcgctgatggcctatggcgtcaccaagatgacgtcgcgcgacggcgatgcctatggcaagggctgcatcgacggcgtcgcggcgccggaggccgccaacaactccgcctcgactggcgcaatgctgccgatgatgacgctcggcatcccgggctcgccgaccacggcggtgctgcttgctggcatggtcatctgggggctcgagcccggaccgctcctgttcacagcgcagcctgatttcgtctggccgctgattggctccttctatatctcgaacttcatcgctgttctagtgaatatcgccttcattccgctgttcctatggatgctgcgcatgccgttcagcatccttgtcccgctggttttcgtgctctcgcttgtcggaacatacgccgcctatatgaacatgttcgatgtctggctgatggtgctggtcggccttggcggcttcttcatgcggatgctcgactatccggtggcgcctgcggtccttgccatcgttctcggcccgatcgccgaaccgacacttcggcagacgctgctacaatatgaaaccttcaccatcttcttcacccgcccaatcgcgggtccgattacggtgaccgcgatcgtcctgatcctgctgccgggcttgcgcctgttgtggcggcgctggcgtgccgcgcgtgttgggacagccggcaactgagccgatccagtctgggtttcacggccttattattttcgcggatgatttaatcccgccatacctacgccatgcccttttaacaacctggcccagtgaacaaccggaggatggcatgctccatttttggttcgcctatcatagcgccgagcaacccgatacacccgaggaaaccgaggtcgagatccagcgctggcgaagctggttcgactggattagtccggcgattgtcgacccggccatcgagctggcaaaggcctgcctgatcatcggcgacggcagcatcgaggttgccaagattcacgaggtatccatgtaggtgacgtcgaaagaagccggcttggccggctttcatcttgccgataacaacagcatgcgaaacaataacgcgtgaaggaggaccatcatgcggatcatctttgtcggtgttggggcgcttggctcatattttggtggcgcgcttgccgaggccggacatgatgtcacgctgctgatccgcaacaaggcgcaccgcgacgccattcgcgcagatggtctgatcctgcatcgtgatggggtggaggcgcgcatcgatcctgtggtggttgatactgaaacagcgggtgatgccggcattgccgacatcgtcattgtctttactaagaccggggccaccaaggccgcgttgcaggcagcaacacctgtcatcgggtcggatacccggctggtcaccgtgcagaacgggcttggcaatgccgaggaccttgccgcattcgtgccgatggatcaggtgatctatggcaccaccatggcccccggcgacatggtcgccccgggcgtggtcagcacgcatggctcccacgtcacgcagttccgtgccgctggcgaaaaccctgttactgagagggtggcaaatgacctcgcagccatgctgagcgctgccggcatcgatacccgcgtcaattcggatgtcgatcgagtgatctgggccaaggtggctttcaattgcgcgatgaacagcctgtgcgcgctgctggatacctctcccgggcagcttcttgattctagtgagacgaaagcgcttgtgaaggcgaccattatggaatgctgtaacgtggcggccgccgtcggggttgaggtggaccgagacggcgtgcatcggacactggacatggtgcatcgtgaacatcgagagcatgtgccctcgatgctggtggatttccgcaaccgccagccgaccgaaatcgcctcgctgaacggcgcggtcgtcgcgcttggggcacgccacggggtgccgacaccgcgaaacgagacgctacttgcgctggtgcgtgcccgcgaggcacagtacctgaaggcataatccaaaatttgaggagagcattatgacgcgcgccgagacacccattccgttggaaatcagctatcccgacacgccaaatgggatgagtgatgttgaatggcatgcccgcgtcgatctcgccgctgcctaccggcttactcaccattatggctggaccagtgttgtctataaccacataactatgcgggtgcctgggaccgatacatttctgattaatccgttcggcctgacctatgacgagatcaccgcctccaacctgacctgtatcgatctggacggcaacaaggtgtccgacgattcctggccggtgaaccgtgccggctatgtcatccatgcagccattcacaaggcgcggcctgatgacctgcattgcgtgatgcacacacatgagcccttcagccagtcgattgctgcactaaatgtcgaggtggtgccgatgatgcaggagggctgtcagctgttcgaacgcatcggctatcatgatttcaacggcattgtccttgacgagaccgagcaggaggcgctgacgaccgccatgggcgctgacaagcacacgctggtgctgaagaaccatgggttgctcaccgccgggccgaatgtggcctgggcgttcgtgcgtcaccagctgttcatccgcaatgccgagatcatgcttcgcaccatgtcgtcaggtgcgcagatcagctatatctctgacgagatcatgcgacatacgcgggagcagttcgagggcggcgcggcgcagtctggggccgctgtgcgccatcctgaatggcccgccttctggcgtatgctggacaggattgatccgtcctggaaacattgatcagctgtcgagcaccgttagcctttcgggaaggccggcgatgacgccacggccggcattggcaaaggcaaaccgcaggaagtcttcctgaccgtcgccaaaaaatgttcccgggatggtcagcacaccggcctcacgcgccatgcgctttgcaacctcgattgatgcggtgccgctaaacgggtgccggacatagccgaaataggcaccggtgcttagaagctcccacccgtcgagctctgccatcacctcactaaacagtgtcgatcgcgcggcgatgcgctggcggttttcctcgcgccagccggcaaggcttgccagcatcggcgtaacggcaatctgaggggtgcgcggcgcgcagatctggatgttgtcgatgatcttggccagctcgtcgacaaaggcggaacccgccgtcaccgcgcctagccggtgaccaggcatgcagtaggattttgagaagctgtagagctggacaagcgtgtcctgccagccgtcccgtgccagcagcccatgcgggcgggcatccgcttctggcaggaagtcacgataggtttcgtcgaggatcagccagatgccgcgtgccttgcaaagcgagaaaatctcgtcgataagctccggcggatagatagagccgcagggattattcggtgagacgatggcgaccgaccttgtgtttgcgccaatggctgcctcgatatcggcaatttcgggcagcatgccgttcgcggcatggcagtcgacatagccgatgccgacgccaagcatgccaagcgcagactcatggttgaaatagcagggacgcgtcatcaggatttcatcgccatgcccggccaccgcgagaacggctgcgacgaacgcctgattgcatcccgaagtgatgagaatctcttccgccacagggctggcgttgtagaaggcgccgagatgggccgcataggcatcgcgaagctcgatgtcaccctcgacccgaccgtagcgggcgctttctgggtctgctgccgcacactccagcgccgctgtcatctctggatgcgccgcataaccagggacggcctggctcatatcaatcagctcgccgagcctgccgtgatacgaggcgcgccagtcctggacgacggataccggcggtgctgtgagctttgatattgctgggttgaacatcttcttcctcgaaataaagctgttactccgagatttaacatctgcctcggccgagacaaagcaaaagcaggttgcctatagccagtcttggcgttaggcatattcagttgaattatctcacgccagcacagatgtctcttaggaaggacatgaacacctgcgttttctttggcagcagtctgcggtctggaaagagaagtgaaaggctggactcctgacgaagctgaagatgcgggaatagttttaccagagagccgtctcggatagcggcgcggcaatagagatcgggcgcaaatgatagcccgccactggtgcaaacgaattcacggagcagaatggggtcattcacctgcatgacctgctcgccttgcagcttgatgtcccttatggcgccgttcttgtccactaccgtcacaggtgtctcaccatatcgtgtctccactgcactgatcaacgatgccagttcatccagccctgggctttctggcagggtagctgcaacctctggtgaggcgatccagatcaacggtgtgttgatcagccgttgctggattaggtctgaatctgtcaccgagccaacaatgacggccatgtcgatggcgtctcggatcagatctggctggccgctgccaatccgaagatcaagccggatatcaggatattccgcctgaaacctggcaaggtgcgggcccacgatttcacgtgcgaacgccatgggaagagcgacgcttagggtgccgcgcggtgtttcgctgagaccggcaagttcggcgctggcagcctcgacttgttccagaatcctgacagcatgctggtagaactggcttccgtcatgcgtcagccgcagggaccggggcccgcgcacaaacaattgcagtcctatatcttcttccagccgggcgattgctctgctgattgttgattttggcttgccaagccttcgagctgctacagagatgccgccaatctcggccacggcaacgaatgcaggtagatgatcaacgttccatttcatgaacgtattgtgcataaaattcacatttgttccacaattggtatcagctatggtgatcgggacgcttcgatttaatccgaagcagggaccggacgacgggcgcctgcaatgacgggggacagcgatgacaagccgagactatgatgatgtaccgggcacctatgttttggatagccgcagctatcgcaagggctatcatctcaacatgttctgcatgtcgctgaacaaagcggaatgccgtgaggaattcgcccgtgacgaaagagcctatctcgaaaaattcccgatgaccgaggaacagcggcaggcggttctggaccgcgactatatcgggctgctgcaacttggtgccaatgtttattacacattcaagatcgtcagccacgacaggcagccgccacaggtgatgtacggcaagatgtcgatgccgccgatgagcttcgaagaatttcaggaaatgatgatcaatggcgggcgtccgatcgaaggcaaccgctcgaaaggggagagctgacatggccagactgatcgccggaatgggaacatcgcatgtgcccggcgtcggcgctgcgatggataacggcaagacgcacgaggattactgggtcgagctgttcaaggggtttgagccgatccgcgcctggcacgccaagaatgtgccggatgtgaatatcatcgtgttcaatgatcacgcgacatcgatgtctctgaaccattactccaccttcatgatgggtgtggccgaacagttccagcccgctgacgaaggctggggcccgcgcaaggtgccgatggtcgagggccatcccgagcttgcctggcatctggtcgagaacctaatactcgacgagttcgacatggcggtcaccgccgatttcgacgtcgaccatggcctgacggtgccgctgtcaatcgcctatgatcagccggacgcctggccggccaaggtgatcccgctctgtgtcaatgtgatccagtatccacagccgacggcgcttcgctgtttcaagctcggccaggccatacggcgtgcggtggatagcttccccgaggacattactgtcgggatctggggaaccggcggcctgtcgcaccaactcggcggcgagcgggcgggtgttgtgaaccccgattttgacaagcggttcctcgacaatctggtcagcgaccctattgcgaacgccagcctaacccataccgatttcatccgtgaagctgggtctgagggcatcgagatgataatgtggaacgtgatgcgcggcgcacttgatgaggatgttcgcgaggtctaccggctatatcacatcccggtatcggcgacagcctatggggcgatcatactcgaaaatagctgatctggttaaacctgtcccgtgaaaggggttcgtgatgcaaacagtggcagtcaggaatttcaggcgcaccgacttaggtcttgctgcaaggctgggagacgcaggagcctcaaccgtccacgaggcgcagggacgaacgggcttgatggacagggcgctccgcccaatctacccaggggcccggatcgggggcacggcggtaaccatccttgccgcgccgggagacaactggatggtgcatgtcgccatcgagctctgccagccgggcgatattcttgttcttgccacgacgtcgccgaacactgacggctatttcggtgacctcctagcaacctcggcacaggcgcgcggggtgaaagggctgatcagcgatgcaggtgtgcgcgatgtcgccgacctgcagcaaatgaattttcccgtctggtcgcgggccgtgtcctcgatgggaacggtgaagaacacccttggcgcagtcaacgtacccgttgtgtgcgctggccagctggtgtttccgggagacgtaatggtggctgatgacgacggggtgacagtcgttgcgcgtgccgttgctgacgaggtcatggaaaaggcccagactagaatcgatgccgaggaagccaaacggcagcgtcttgctgctggcgagcttgggctcgatatttacgatatgcgtggtcggttggccgatgccggcctcacctatgtcgagacactcgacgaactgggtgatttcaacgggattgactgccgcggccgatagcctgaatttcacattgattcacttgatccgcttgcacatatattctgattggcagcgaactttttggtcgtcgactttgattaacgcatcttttccgccgctttgccgcttatagaagcagttagtaccaccatcaacatattcgcgacggcgcaaacggcatagttcaaatttgggcttatcttcttcattctgcgacttgtatgtccggccggcggcgccggcacggccggcaaaagccgtggtcagcagagcgatgccgctggaaaggaaaagccggcgatcagtcttcctgggtttcgatgtcatcttctgatcccggtagataggctttgccctgaatcgcgcgtgcaccaagcgtttcaaaaagttccttattgttggcgatgagagcgcgaagcgtctcatcaactttttgctcggcctcttctggcgttgcgacatccatttcggcaacgccgtcaataatccaatgaactttaactttcatttttaatctccctgccgtcagtcaaagctgatgttaaggctttcataaatcgtgcgagcctggctttgcagatcgacgagcttttcatcatctaaactgtcccg

SEQ ID NO: 3 - DNA, 7629 bp, Unknown
  uncultured marine microorganism HF10_19P19
ggacgttctagaacgcgacgtttggggagtgtcctatgtcaataattaagtctttaaagtcagtcgctattgcatcactggctattctcattccatcaattgcattagcagctggtggaaacttggagccgaacgatcctgtcggcattaccttttggttgatatcgatcgcaatggtcgccgctaccgtatttttcttgatggaatcattgagagttgacggcaagtggagaacatcaatgattgtgggcggacttgttacactagtagctgcggtccactacttttatatgcgtgatgtttgggttgccacaggtgcatcgcccacggtctttcgttatgtagattggctaatcacagtgcctctgcagatgattgaattctaccttattttagctgcttgcactgccattgctgtcggcgtcttctggcgcctcatgatcggcacaatggtaatgttaattggcggatatctaggtgaggcgggcttcatcaacgctacggttggtttcgtgatcggaatggccggttggggatatatattgtatgaaatctttgctggtgaagctggcaaggttgctgcagaaggtgcgcccccatctgtacaatcagctttcaacacaatgcgtctgattgtgactattggttgggctatttatccacttggatatttctttggctacatgaccggaggagttgacgcaaattcgctgaacctgatctacaacgttgctgacgttgttaacaagattggtttctgtttagcaatttgggctgctgctacttcacaatctgaagcggcaaagtaataaacttatcgcaagcatccataatttaaatgggtgcttgcgctttattcagcttaattatgtgtttctcaatttttttgcacctttcatcttaaaatatctacaaaagaagggccggaagatgacagagaacatagccagccaagtcaatgagatgcagggcgcagaaaagcttgatgctcttcctgatttgtctgctgtactgtcagcctttattgtgcggagattgccatcgggcaaacacccactcgccactgctgctcaacatcatttcaaacgtactggcaaacagttgcgtggccgtatggctctagccgctggagacagttttggcgtaccacgaacggccagcctaccctgggctgcagctgtggaagttcttcataatgcttcactggtgcacgacgatatctgtgatggagatatggtgcgccgtggcgcgccgtctgtctgggcattttatgggcgcgatacggcactggcgcttggtgactggctaattgcgctctcctttgagctggcagcagaagctgcctatctcgggcagacgccacagctggtttctatcctgtcacgccacatggccgccaccacggcaggacaagctctcgagtttgaggttcgcgcctacccagactggtctggctatatggacatcagcacaggcaaaactgcccctttatttattgcatcggttgaagggattgctcacctggccggccgccgtgatgcaatacggccgctgaaccggtttttcagtgctgcaggcggttgttatcaaattgccaatgatatgttgaatgtgattggtaaggatggcgcggaaagcccggcatccgacctgctgcgtcgcgccccgaacgcggtgattgtgatgtttcagacaacattggataagcatacagcgacagcctttgacaagtggctgtcctcaggcgacacacatggtgcgcttacctggcaggaacatgtgcgccggtcaccagcgctgaccatgacttcatcagccttgctgagcatgctggaagaagcagaagcatcatcggctgcctttcccacagattgccgggcaatcatcacaccaatattggcacagcttcggcatgtctgccgcgatcttaccagtttaaatgcctaagtgatatccaataaaatgtttgggcaggacagacaggcatatcctgtgctagactggaggaaaccggctagaaaatgaggatgtggcaaatggatgatgcccgcccctactcctataagaaagatatggtagagcgcgttcatagcgtggtcgtcggcagtggttttggcggaattgctgcagccttgcgtatgcgtgcaaaaggccatcaggtaacgctgatagagcggctagaagatgttggtggccgcgcgcgtgtatttgagcgtggcggctttcgccatgatgcaggcccaactgtaatcaccgccccatttttgtttgatgagctgtttgacctgtttggagagacgcgttctgaacatctgaaatttgtgccgcttgatccatggtacaggtttcatttccatgatggatctgagtttgattaccgcccgtcactagctgacacacatgcagagattgctcggttttcgccagaagatacaaaaaactatgacaggttggtgaacacgtccaaacagatttttgatgtaggttttacccaactggcagataagccgtttacccgttttacctctatgatcaagctaatcccgcaattattgcggctacgcagctatttgacagtagcccaactggtaaacagccacatcaaacaccccttattgcggcaggccttttccatccatccgttacttgtaggcggcaaccccttcgatacgacatctatttatgctttgattcattatctggagcgcaaatggggcgtgtatttttgcatgggcggtacaggcaagctggttgctgaattgaaagcacttatgatccgccagggtgttgatctgaaactgaatacagatatcattgagatttgtgtttctgacaagaaagttacaggtgtgcgcacggccgacggggcctttatttctgctgataatgtgatttgtaacgctgacccaccaactgtgtaccggcagatgctgcctagccagacccacctccgcaaaaaggtgctgccagaggcagtaacccgctattcgatgggcctgtttgtgctgttttttgggacccagcagcgctatccagacattgcccatcacacgatatggatgggaaaacggtataaggatttactgcatgatatctttagcaaaaagatattggcagaagatttttcattatatctgcatcgcccgaccgccacggacgcccgttttgcacctgagggctgtgatagtttttatgtcctctgcccggtccctaacctgcagggtgatgtggactgggcaaaacaagggccactcctgcaagacaggattgtcaccgctcttgagaaaagtattatgccagacctacgtaatgtgattaccgaagatttttatatgacacctgaagatttcaaagcagattaccgatctgaacatggtgctggcttttctatatcccccagcttcacccaatctgcctggttccggtatcataaccgtgatccgcatctggcaaatctgtatttttcaggagcaggtgcacatcccggtgctggtatgcctggcgtgctctgttcagccaaagtcgttgaaacgttggtcagcgaagatgaacggaaagccgcacatgtccagcccacgcgctgaacagaaacagcaagacgcacattatccagctgagagaggcacagccctaaagacactggctaaaaatggcaaaagcttttactgggcatcacgcctgctgggccgccagatggcaatagatgcagctgagctgtatagcctgtgccggcttttggatgatattgccgatggcgatatcaacgggctggatacgaaagaaggaacacatcgactccgccatatccgccgccaactgacccaaatagatgctgggaaaatacctgaacagccagacccagcacttttgcagtatatgcaggttatgaaccgatgccagatcccggttatgccacttatccatctattggacgggctgttgctggatcagtctgcaatgctgcttaaggatgaggcagatttggtggcttatgcttatcatgtagcaggcgctgtcgggcttctgatgtgtcctgttctgggctgtgacgacaggacagccttccgttttgctgtggatatgggcatcggcatgcaactaacaaatattgcccgagatattctggaagatgcccagatgggacgtcgctacctgcctgaaagctggacaggccagctctcgcctgaccagattgttgcctgtgcagaggcccctgaatcgcagaactataagcagattcaagctgccgcagacaggctgctgactcttgctgaccagtattatgaaagcgggcgtctggggctaggatttctgcctgtgcgcgcacgatattgcattgctgttgcagcaggtgtttatcgcgccattgggacaaaattaagaacccgtaagctgcggtggggcgatgggcgcgtggtaactagcaagagcgagaaactgtctgctagcctgcaggccctatccaaactctatcgcctgccaaaagctactcataacaccaatttgcatgcgcctcttaccagtttattagatgaggcacttaaatgatcagaacagcctccctagaacagacagaattggcaattattggagcaggctgtgccggcctgtcgttggcccgccagctggtgcggacagaaaagacatcaattataaaggcttgtctgtatggtcctgtctctcccgccgcaacaaataggcatagttggggattctgggcaacacagggacttaaagagcagaccgagctctcaagacaacgctggcataaatggcagattatcacagcagaacggaaggttacgcaaacagctgattctcatccctactgccgtctcgatagccgtgattggctgacccattgtctgaacattatagacggtaatattcagcaaaagcctgacaacctgcctacacccggcgcccaacctgtctttgacagccgccctccaaatgtgcctgagggggccttgctgcagcattttaagggtgtggagatcaaagcagcacaaccttgtttttcacctgatacggctattttaatggatttcagatgtgaccaatcccgcggcattcatttcatctatctgcttccttacagccctacggaggcattagtcgaatctaccatgctttcccctaaacggcagaacgatgacttttatgcagaggccataaaaacctatttggaaacttattgggcgactggcggctggcaagtcaagcatactgagcagggctgtattccgatggcttttgtacaaccagtcaatctcgattatctgcccatcggtgcaaatggcggttgtgttcgcccgtcatctggctatgcgtttgcctttatccaaaaacatacagatgctattattgacagactggtcctcagtggtacagaccccttaacgccagcaacaatgcctcgaccaatcagtcgttttgatctgtttctggacagaatctttctgcacgtcatccggcatcaccctgaaaaggcagctgagttgtttgcgcagatcgcccttgccctgaacggcgatgaattcgcccgctttatgtctggccttgcagatttcaaaatctatgcaaaactgatcacggcaatgccagccggcctgtttttacacgcattgtggaatatacatgtgaccaacagagataacgcggataaaaaataatgcgagcaaaacgcctctagacggcgtagattcagagtaaatatgatagatatcttatcagtaacagacctatttgcccttgcagcggtgatgctgattggtttgccccatggtgcatttgacggcgcccttgccttttgtttaggctttgggcgcagtcctggcaaaattattggttttctggtcatgtatctgttattggccgggttgtctgccttgatctggcttgtatcacctgtcttcgccctagctggttttttggtgctaaccattgtgcattttggtagcggcgatacagaacatctgtttcaaccaggcccgcgcgttgtccagcgcagcctgaaagcctgtcagattctggtgcatggtggcatggtgactattttattgcctgtgtttcacacaacagaggtgagccaactgtttattgttcttgccgggccaaatgcggttctgatcattgatgcgctgagaccagcgctgaaaatctggcttgcagctgcctgtatttacgctagcgcagcccttttcaaacgccaatatgctgctgcggcaggtgaactggctgggctggctgtgctagtgtggctattgccgcccttagcaggttttgcggtttatttctgtattgtacattcacgccgtcattttactagtatctggaaagctatgcagctctttgtcagccgccgcttcattctaatcagcggcggaattctgaccgcagcatcctgggcgatgggcgcagggctatattttagccagacaatgtcaggcagtttttcaccagatgaagcctttatccgcacagtctttatcctgttagctgctttgacggtaccgcatatgttgttggtggatacaatgtaccgccccacattgaaaaagattacacagccatgacccgaacaagcacgccgctaacaactgatcgtaaaaatgcgcatcttgatctggccaagaccagccagcccctcgccgaccacccgctggatgcggtgagcctgccttattgcgcgctaccggaatgtgatttaaacagagtcagccttaccacagaatttctcggaattgagctggattccccgctaataataacaggcatgactggcggtacagaccgggccatggcgataaacagggtactagcagataccgcccagaagaaaaaggttgcgcttggacttggatctcaacgtgccagccttgaatcagggcaaagtcaggctgagctgcgccgtctggctcctgatgctgttctgattggaaacctgggcggcgcgcagctcgctggaaaggacggactgaagctggcccgcgctgctgttgaagatatcagagctgatgcgctagcaattcatttaaaccccttgcaagaagccattcaaccggaaggtgatcatgactggcgcggggtattatcggcgattgaaacggctgttggtacgctgaattgtcctgtgctagttaaagaggtgggagcaggcctgtctggtaatgtagtgcgacggctggcagccattggtgtacggcatgtggatgtcgcggccagaggcgggacaaactgggcccaaattgagcttaacagacgcccggaaactgaccgagcgcattatgcgccctttttatcctgcggcctgatgttgcctgatgcgattgcgcaggcccgggctgtatccaaccacctgtgtataattgcctctggtggagtccggcatggcttggatgcggcgaaatgcttgtggttgggagcagatttagttggcatggcaggtcatatcctgcgtacagtagaagataacgctggtcatctgcatccaaaacagttgagcgacttgctgtataccgtgcaacagcaattgcgcttaagcctgtttctagccggaaaatcatctataaaagccttcaaaagggaataaaagaccaattccacttgcaattctgcaatctaggc

SEQ ID NO: 4 - DNA, 7470 bp, Unknown
  uncultured marine microorganism HF10_25F10
atgaaactctcaatgggtaaggtgatgaagactgcgatgatcgcaggcggactcgccactctcccaactgtcgcaaacgctgctacggcgatgcttgctgcaactgactatgtaggcatctcgttctggaccatttcgatggccatgatggcagccgcaatcttcttcctgatggaagcaggtcgcctgcctggcaagtggaagaccgcaatgacgattggtgcactcgtgcagatcgttgcagcggtacactatttctacatgcgcgacgtatgggtcagcactggtgaaacgccaaccgtttacaggtatgttgactggttgatcactgttccgctccagatggtagaattctacttcatcctggcagcggtggccgttgttagcgctggtatgttctggcgcttgatgatcggttcgctggtaatgctggttgctggttaccttggtgaagcaggccttgtgaatgcatggctcggttttgtcgtaggtatggccggatgggcatacatcctctacgagatctttgctggtgaagctggcaaggtgtccgcagagtccgcgccggaggctgttcagaagtcgttcagcacgatgcgtatgatcgtgaccgtcggctgggcaatttatccgctcggctacttcctcggttacctgaacggtgctgctgacgctgtcaccttgaacgttatctacaacgtcgctgacgttgtaaacaagatcgcgttcgttggtgtgatctgggcagctgccaacgcagaagccgcgaaagcctaaatgcattgatgaagggcggcacccacacgtcgcccttcatcacatttccttaatgacattttgtgaatcatcttgttgcgcgggaaggtgcggatattatgactgacctgtctacaccctcgaaacatgcatttgtcgagcagggttccgggctttccatttcgaagtccacttccgagattattcacgatttcatccttgaaagaatgcctgcccccgggcagacgctcgccgacgcggctatgcatcatttcgctgccccgggcaagatgctccgcgcaaagatggcgctccgtgctgccgaccttctcaaggtcgaccggccggcggcccttcactgggcggcggcgatcgaggttctccacaacgcgtcacttgtccatgatgacatttgcgacggcgaccagttgcgccgcggacgtccggccgtctggtcgaaattcggacgcgatgtcgcgctcaccctcggcgactggcttattgccctatctttcgagctcgccgcagaggccgcgcaacgcgcgcagacgccgatgctgctgaagattcttgccacccatatgaagacaaccactgtcggcgaggcgcacgaattcgacccggcgccggttaccgactgggatcattacctgcagggtgccggtgacaagaccgcgccgctgctgaccgcgcctctcgaggggatcgccgccatggccctccatggtggcgccgctccgacgatcggtgcctgcttccgctcgcttggcaatgcctatcagatcggcaacgacatcctgaatttctcgggaagcgacggtgccaatacctgcgggtcggacatggctcgccgtgccccgaacggcgttgtcgtccttttccgccagacactggatgcagaccgtcttgccgcattcgacagctggtatgccagcggctcggatgacggattcgatgcgtggctcgcagagctgcggcgctcgcccgccctgttcgaggcggcgcggcggatgaatgcgatgctcgacgacgcgacgggtcttgccgacacgctgccgcccgaaatggcagcggtgatcacgcctatacagcagcttctgcagcaggtatgtctgaagtctgtggcttcgatcaaacaggcctgacgcaagcattatggcaccagccccctcggctattcaggatctggcggccccgcattccaagaccgggccagccgaccagagcgatgttcttgtaatcggtgccggttttggcggaatcgcggctgccctccgtatgcgcgcccggggctattccgtgacgcttgtcgacaggctggatgccattggtggacgggcgcaggtgtttgaacggggcggcttccggcatgatgccggcccgaccgtgatcacagctcccttcctgttcgacgagctgttcgagctgttcagagagcgccgcgagaatcatctcgacttccggccgctggacccctggtaccggttccatttccatgacggcaaccagttcgactaccgcgccacggtcgaggacaccaacgccgagatcgagcgtttctccccgcgtgacgtcgccggatatgcagggctgctggaggcatcccggcagattttcgaggtcgggttcgagaaactcgccgacaggccctttactcgtttcatgacgatgatggcgcaagtgccctcgctgttgcgccttcgcagctatcacaccgtcgccagcctggtgaaccgccatatcaaacatccactgcttcgccaggccttttcgattcatccgctgcttgtcggcggcaacccgttcacgaccacctcgatctacacgctgatccattatctggagcggcggtggggtgtgttcttctgtatgggcggcaccggcaagctcgtcgaggaactgcaccggcttcttgaacgcgccggcgtcaatgtcgagcttggcgtggatattgagcgcatcaccatgaaaggcggccgcgccaccggcgccgaatccacagctggcaggcgctatattgcggatcgggtgatctgcaatggcgatccgccgaccgtctatcagcagatgctgcccaacgagacacgctccaagcgggcaattcccgaagccgcgacaaaatattcgatgggtctttacgtgatgttctttgggacacgcaagcagtatcccgacgttgcgcatcataccatctggatgggaccgcgctatcgtgagctgcttgccgatatcttcgaccgcaagattcttgccgatgatttctcgctctatcttcaccggccgaccgccaccgacagcagcttcgcgccggacggctgcgacagtttctatgtgctgtgcccggtgccgaacctgcttggcgatgtggactgggagtttgagggaccaaggcttcgcgaccgcattgttgcggcgctggaagagacgatcatgccgggcctgggcgcgtccattaccgatgatttctggatgacgccggaagatttcaagcgcgactaccgttcgatgcatggcgccggtttctcgatcgcgccgattttcacgcaatcggcgtggttccgctaccacaaccgcgacccacatatcccgaacctgtatttttcagccgccggcgcgcacccgggagcagggatgccaggcgtcttgtgttctgctaaggttgttgagcgactgattgaggaggaagaccgggcggccgccgccagcggaaggctggcaaagggggcctgacggcgggagccggcactcatgtcgaacgaagagctgacatccgaaaaatcgcttgccgccaacggcaagagtttccactgggcacgccgttttctgggacagcgcatgggaagcgacgcggcctcactctatgcgttctgccggcttctcgacgatatggccgacggcgatatacagaacggtccgaccaggcttgccgccatccgtgccgatctgattgccggccgcgagggggctgatcccgcattccttgcattcctgccgctgatgcgggaaaaacgctttccaacggacgtgcttgtggcgctgatcgacgggttgctcgaggatcaggccgaagaggtggcgctcgccgacgaggccgcgttgctgcgctacgcctaccgggtggcgggaaccgtcggcctgctgatgtgccatgttctcgactgccaagcgccggcagcacgggcgcatgccatcgatctcggcatcgccatgcagctgacaaacatcgcccgcgacattctcgaggatgccggcatgaaccggcgctatgttccaggtgactgggtcggccatgcaagcccgcacgaaatccgtgccgcggcaaggtcgccggaaagcatgctggcgcgcgatgtgaccgctgcggcaggccggcttctggatcttgccgagaccttttatgccagcggggcccgggggtatcgctatctgccatggcgggcgcatctcgccatcggggtggcggcacgggtctatcggcagatcggcatccagcttcgcggtgccaaccttgcctggcatgccggaaggcaggtgaccggcaaggcggccaagctgcgatgctcgctgctggcgctgggaagttttgggcgtcggatcggcatcgggacatcgccacatgacgaccgccttcacgcagcactgcgggggctgccctatgtcgcctgaccacgccatgcggcaggttcgtgtccttggtgacggctgcgccgcgctgtcgctggcggcgcgcgccaatgaactggcgggccataggctgacacttgtcaggccgcgaggcgccccgcaggagacggaccatatctggggtttctgggacggaccgaaccttgccgccgccagccgccttgcccgcaagcgctggcacagctggtcgatcgtgacccgccggggaaaggctgtattgcgatcacatgaacggccctacacggcgcttcaccggctggactggacggcgcattgccgtgaagcggccagacgtgccggcgttgcaatcctgaccggcggggaagctgcgacagcggcagaccaggtcttcgacagccgcccgccgccagtgccggacgacataatgctgcagcattttcttggcctcgaagtgcgaagcaagcgtgcgattttcgatccggataccgccattttgatggacttccgtgtggaccagtcgcgcggcatgcatttcatctatcttcttcccttttccgcgaccgaggcgctggttgaatccaccctgttcacgccatctgtctgtccgaagagctattatcgcagggaaatcgtcgcctatctcgaggcccatttcggactcgatgaattcgagacgcttcgcaccgaacgcggggtaatcccgcttggcatcttgccgccgcgcgatccgtccattccgggtgtaggcggcaatggtggcgcgatccgcccgtcgagcggctatgcctttccctttatccagaaacagatcgaccgtgccatcgccgcggcatgtgaggggcgcgagctcggctttgctatgccgcaccgccgtgttgatttgtggatggacgcggtgctgctgaccgttctgcgccactggccggaacaggctcccgaactgttcctgcggatggggcgtgcgctcgacggtgacgagttcgcacggttcctcagcggcgaggcggacttgccgctccggctgaaagtgatcatggcgatgccgaaggccccgttcctgcgcgggcttgtccgccttctggccggcagccggccacaacgcacggcagaggcgggatgatggaaatgatgacgtccatagcggcgatggcgccgctcgaccttgcggcgctggccgccatagtgctgatcggcctgccgcatggcgcgctcgacggggcgatcgccatccatctcggcttcacccggaaaattctgcatttcatttggtttctggcgctttatatcgcgatggccggggttgtcgtcgcggcatggctgctggcgccgaccgcctgcctgctgggcttcctgattatcagcatgctgcattttggtgctggtgacgcgcgccacggaaccggctggctccgcagtgccgagattctggcccatggcggccttgtcatcgtcggcatcagccagatgcatcatgtagaggtagatgtcatctttggttatctgaccgggcgcgatacggcgctcctctggcaaggcatcgatgttctgacgttgattgtcgggatggctgttgtcgtctgccttgcccaggcgctctggcatcggcgctggcgcgggacggctttcgagctcggcgcgcttgcggtactgtttgcgttgacaccgccattggtcggttttgccgtctatttctgctgtgtgcactcggcaaggcatgtctacggcatcgttaacagcttgcggcgcgaaatctcgcggttcagcctgctcaaccaggccgctgccttttccgtggcaagttgggttgccggcggtgccgccatcttgtggtttgccgatatgtccaatcctgagcctttcgtgctacgggtggtgtttatcggccttgcggcgctgacggtgccgcatatgattcttgtcgatggtttcttcaggcggtccggcaggaccctgaagcgccagtttatcagccggtaggtgcctatggatgatatgcatgaaaacgctggaacaccgtccgcaggcgatcgcaaggatgcgcatcttgctctggccgccgacgcattgtcccgctccgggattagtgccggcttcgagcgtgtgcggctcgagcattgcgcccttcccgagtgcagccttgccgatatcgatatcaccacttcctgccttggcaggcccgtcgaggcaccgctgttcatcgggtcgatgacgggcggcacggcgcatgccgatgcgatcaatgcggtgcttgccgacacagccgaggccaccgggatcgcgctggctgtgggctcgcagcgcgccagcattgaatcaggccgttcccaggcggtactgcggcagcgcgcgccatcggtgccgctgatcggcaatctgggcggtgtgcagctcgccgcgccgggtggtattgatctggcatgccgcgccgtggtggatatcggcgccgatgccatcttcatccatctgaacccgcttcaggaggcggtccagcccgagggcgagacggactggcgtggtgttctggacgcaatcgagacccttgtcggtgtgctggaggtgccggtgatggtcaaggaggtcggcgccgggatcgggcccgatgttgcgcagcgcctgtttgatgccggggtccatgctgttgatatcgccgggctgggcggcacgaactggacacgtatcgaggcggcgcgccgcgaggatgccgccctgttcgagccgttcctcgactgggggttgccgacggtagatgcgctccgcgctgtcaggagcgcctgcccgaacgccaggctgatcgcatccggtggtgtggaaaacggtctcgacgccgccaaggcattgtggcttggcgcggcgctggtctcgatggccggaccagtgctgcgcgtgctgactggcgacgggcgtggtgcacctgacggggccgcggcggtgcatgtcatcgaacgctggaaatcgcagcttcgcctggcgctgttcctgaccggtgcgcccgacctgccgtcatttgctggcaagcccggtcatgtcggtgggccatatcagagggccattggtgcgcagaaccggtga

SEQ ID NO: 5 - DNA, 29 bp, Artificial Sequence
  Synthetic Oligonucleotide
ggacgttctagaacgcgacgtttggggag

SEQ ID NO: 6 - DNA, 33 bp, Artificial Sequence
  Synthetic Oligonucleotide
gcctagattgcagaattgcaagtggaattggtc

SEQ ID NO: 7 - DNA, 7646 bp, Artificial Sequence
  uncultured marine microorganism HF10_19P19 with flanking vector sequence
ggatcccacggacgttctagaacgcgacgtttggggagtgtcctatgtcaataattaagtctttaaagtcagtcgctattgcatcactggctattctcattccatcaattgcattagcagctggtggaaacttggagccgaacgatcctgtcggcattaccttttggttgatatcgatcgcaatggtcgccgctaccgtatttttcttgatggaatcattgagagttgacggcaagtggagaacatcaatgattgtgggcggacttgttacactagtagctgcggtccactacttttatatgcgtgatgtttgggttgccacaggtgcatcgcccacggtctttcgttatgtagattggctaatcacagtgcctctgcagatgattgaattctaccttattttagctgcttgcactgccattgctgtcggcgtcttctggcgcctcatgatcggcacaatggtaatgttaattggcggatatctaggtgaggcgggcttcatcaacgctacggttggtttcgtgatcggaatggccggttggggatatatattgtatgaaatctttgctggtgaagctggcaaggttgctgcagaaggtgcgcccccatctgtacaatcagctttcaacacaatgcgtctgattgtgactattggttgggctatttatccacttggatatttctttggctacatgaccggaggagttgacgcaaattcgctgaacctgatctacaacgttgctgacgttgttaacaagattggtttctgtttagcaatttgggctgctgctacttcacaatctgaagcggcaaagtaataaacttatcgcaagcatccataatttaaatgggtgcttgcgctttattcagcttaattatgtgtttctcaatttttttgcacctttcatcttaaaatatctacaaaagaagggccggaagatgacagagaacatagccagccaagtcaatgagatgcagggcgcagaaaagcttgatgctcttcctgatttgtctgctgtactgtcagcctttattgtgcggagattgccatcgggcaaacacccactcgccactgctgctcaacatcatttcaaacgtactggcaaacagttgcgtggccgtatggctctagccgctggagacagttttggcgtaccacgaacggccagcctaccctgggctgcagctgtggaagttcttcataatgcttcactggtgcacgacgatatctgtgatggagatatggtgcgccgtggcgcgccgtctgtctgggcattttatgggcgcgatacggcactggcgcttggtgactggctaattgcgctctcctttgagctggcagcagaagctgcctatctcgggcagacgccacagctggtttctatcctgtcacgccacatggccgccaccacggcaggacaagctctcgagtttgaggttcgcgcctacccagactggtctggctatatggacatcagcacaggcaaaactgcccctttatttattgcatcggttgaagggattgctcacctggccggccgccgtgatgcaatacggccgctgaaccggtttttcagtgctgcaggcggttgttatcaaattgccaatgatatgttgaatgtgattggtaaggatggcgcggaaagcccggcatccgacctgctgcgtcgcgccccgaacgcggtgattgtgatgtttcagacaacattggataagcatacagcgacagcctttgacaagtggctgtcctcaggcgacacacatggtgcgcttacctggcaggaacatgtgcgccggtcaccagcgctgaccatgacttcatcagccttgctgagcatgctggaagaagcagaagcatcatcggctgcctttcccacagattgccgggcaatcatcacaccaatattggcacagcttcggcatgtctgccgcgatcttaccagtttaaatgcctaagtgatatccaataaaatgtttgggcaggacagacaggcatatcctgtgctagactggaggaaaccggctagaaaatgaggatgtggcaaatggatgatgcccgcccctactcctataagaaagatatggtagagcgcgttcatagcgtggtcgtcggcagtggttttggcggaattgctgcagccttgcgtatgcgtgcaaaaggccatcaggtaacgctgatagagcggctagaagatgttggtggccgcgcgcgtgtatttgagcgtggcggctttcgccatgatgcaggcccaactgtaatcaccgccccatttttgtttgatgagctgtttgacctgtttggagagacgcgttctgaacatctgaaatttgtgccgcttgatccatggtacaggtttcatttccatgatggatctgagtttgattaccgcccgtcactagctgacacacatgcagagattgctcggttttcgccagaagatacaaaaaactatgacaggttggtgaacacgtccaaacagatttttgatgtaggttttacccaactggcagataagccgtttacccgttttacctctatgatcaagctaatcccgcaattattgcggctacgcagctatttgacagtagcccaactggtaaacagccacatcaaacaccccttattgcggcaggccttttccatccatccgttacttgtaggcggcaaccccttcgatacgacatctatttatgctttgattcattatctggagcgcaaatggggcgtgtatttttgcatgggcggtacaggcaagctggttgctgaattgaaagcacttatgatccgccagggtgttgatctgaaactgaatacagatatcattgagatttgtgtttctgacaagaaagttacaggtgtgcgcacggccgacggggcctttatttctgctgataatgtgatttgtaacgctgacccaccaactgtgtaccggcagatgctgcctagccagacccacctccgcaaaaaggtgctgccagaggcagtaacccgctattcgatgggcctgtttgtgctgttttttgggacccagcagcgctatccagacattgcccatcacacgatatggatgggaaaacggtataaggatttactgcatgatatctttagcaaaaagatattggcagaagatttttcattatatctgcatcgcccgaccgccacggacgcccgttttgcacctgagggctgtgatagtttttatgtcctctgcccggtccctaacctgcagggtgatgtggactgggcaaaacaagggccactcctgcaagacaggattgtcaccgctcttgagaaaagtattatgccagacctacgtaatgtgattaccgaagatttttatatgacacctgaagatttcaaagcagattaccgatctgaacatggtgctggcttttctatatcccccagcttcacccaatctgcctggttccggtatcataaccgtgatccgcatctggcaaatctgtatttttcaggagcaggtgcacatcccggtgctggtatgcctggcgtgctctgttcagccaaagtcgttgaaacgttggtcagcgaagatgaacggaaagccgcacatgtccagcccacgcgctgaacagaaacagcaagacgcacattatccagctgagagaggcacagccctaaagacactggctaaaaatggcaaaagcttttactgggcatcacgcctgctgggccgccagatggcaatagatgcagctgagctgtatagcctgtgccggcttttggatgatattgccgatggcgatatcaacgggctggatacgaaagaaggaacacatcgactccgccatatccgccgccaactgacccaaatagatgctgggaaaatacctgaacagccagacccagcacttttgcagtatatgcaggttatgaaccgatgccagatcccggttatgccacttatccatctattggacgggctgttgctggatcagtctgcaatgctgcttaaggatgaggcagatttggtggcttatgcttatcatgtagcaggcgctgtcgggcttctgatgtgtcctgttctgggctgtgacgacaggacagccttccgttttgctgtggatatgggcatcggcatgcaactaacaaatattgcccgagatattctggaagatgcccagatgggacgtcgctacctgcctgaaagctggacaggccagctctcgcctgaccagattgttgcctgtgcagaggcccctgaatcgcagaactataagcagattcaagctgccgcagacaggctgctgactcttgctgaccagtattatgaaagcgggcgtctggggctaggatttctgcctgtgcgcgcacgatattgcattgctgttgcagcaggtgtttatcgcgccattgggacaaaattaagaacccgtaagctgcggtggggcgatgggcgcgtggtaactagcaagagcgagaaactgtctgctagcctgcaggccctatccaaactctatcgcctgccaaaagctactcataacaccaatttgcatgcgcctcttaccagtttattagatgaggcacttaaatgatcagaacagcctccctagaacagacagaattggcaattattggagcaggctgtgccggcctgtcgttggcccgccagctggtgcggacagaaaagacatcaattataaaggcttgtctgtatggtcctgtctctcccgccgcaacaaataggcatagttggggattctgggcaacacagggacttaaagagcagaccgagctctcaagacaacgctggcataaatggcagattatcacagcagaacggaaggttacgcaaacagctgattctcatccctactgccgtctcgatagccgtgattggctgacccattgtctgaacattatagacggtaatattcagcaaaagcctgacaacctgcctacacccggcgcccaacctgtctttgacagccgccctccaaatgtgcctgagggggccttgctgcagcattttaagggtgtggagatcaaagcagcacaaccttgtttttcacctgatacggctattttaatggatttcagatgtgaccaatcccgcggcattcatttcatctatctgcttccttacagccctacggaggcattagtcgaatctaccatgctttcccctaaacggcagaacgatgacttttatgcagaggccataaaaacctatttggaaacttattgggcgactggcggctggcaagtcaagcatactgagcagggctgtattccgatggcttttgtacaaccagtcaatctcgattatctgcccatcggtgcaaatggcggttgtgttcgcccgtcatctggctatgcgtttgcctttatccaaaaacatacagatgctattattgacagactggtcctcagtggtacagaccccttaacgccagcaacaatgcctcgaccaatcagtcgttttgatctgtttctggacagaatctttctgcacgtcatccggcatcaccctgaaaaggcagctgagttgtttgcgcagatcgcccttgccctgaacggcgatgaattcgcccgctttatgtctggccttgcagatttcaaaatctatgcaaaactgatcacggcaatgccagccggcctgtttttacacgcattgtggaatatacatgtgaccaacagagataacgcggataaaaaataatgcgagcaaaacgcctctagacggcgtagattcagagtaaatatgatagatatcttatcagtaacagacctatttgcccttgcagcggtgatgctgattggtttgccccatggtgcatttgacggcgcccttgccttttgtttaggctttgggcgcagtcctggcaaaattattggttttctggtcatgtatctgttattggccgggttgtctgccttgatctggcttgtatcacctgtcttcgccctagctggttttttggtgctaaccattgtgcattttggtagcggcgatacagaacatctgtttcaaccaggcccgcgcgttgtccagcgcagcctgaaagcctgtcagattctggtgcatggtggcatggtgactattttattgcctgtgtttcacacaacagaggtgagccaactgtttattgttcttgccgggccaaatgcggttctgatcattgatgcgctgagaccagcgctgaaaatctggcttgcagctgcctgtatttacgctagcgcagcccttttcaaacgccaatatgctgctgcggcaggtgaactggctgggctggctgtgctagtgtggctattgccgcccttagcaggttttgcggtttatttctgtattgtacattcacgccgtcattttactagtatctggaaagctatgcagctctttgtcagccgccgcttcattctaatcagcggcggaattctgaccgcagcatcctgggcgatgggcgcagggctatattttagccagacaatgtcaggcagtttttcaccagatgaagcctttatccgcacagtctttatcctgttagctgctttgacggtaccgcatatgttgttggtggatacaatgtaccgccccacattgaaaaagattacacagccatgacccgaacaagcacgccgctaacaactgatcgtaaaaatgcgcatcttgatctggccaagaccagccagcccctcgccgaccacccgctggatgcggtgagcctgccttattgcgcgctaccggaatgtgatttaaacagagtcagccttaccacagaatttctcggaattgagctggattccccgctaataataacaggcatgactggcggtacagaccgggccatggcgataaacagggtactagcagataccgcccagaagaaaaaggttgcgcttggacttggatctcaacgtgccagccttgaatcagggcaaagtcaggctgagctgcgccgtctggctcctgatgctgttctgattggaaacctgggcggcgcgcagctcgctggaaaggacggactgaagctggcccgcgctgctgttgaagatatcagagctgatgcgctagcaattcatttaaaccccttgcaagaagccattcaaccggaaggtgatcatgactggcgcggggtattatcggcgattgaaacggctgttggtacgctgaattgtcctgtgctagttaaagaggtgggagcaggcctgtctggtaatgtagtgcgacggctggcagccattggtgtacggcatgtggatgtcgcggccagaggcgggacaaactgggcccaaattgagcttaacagacgcccggaaactgaccgagcgcattatgcgccctttttatcctgcggcctgatgttgcctgatgcgattgcgcaggcccgggctgtatccaaccacctgtgtataattgcctctggtggagtccggcatggcttggatgcggcgaaatgcttgtggttgggagcagatttagttggcatggcaggtcatatcctgcgtacagtagaagataacgctggtcatctgcatccaaaacagttgagcgacttgctgtataccgtgcaacagcaattgcgcttaagcctgtttctagccggaaaatcatctataaaagccttcaaaagggaataaaagaccaattccacttgcaattctgcaatctaggcgtgggatc

(19) United States
(12) Patent Application Publication
(10) Pub. No.: US 2009/0233342 A1
(43) Pub. Date: Sep. 17, 2009

(54) HETEROLOGOUS EXPRESSION OF PROTEORHODOPSIN PHOTOSYSTEM

(76) Inventors: Asuncion Martinez, Arlington, MA (US); Edward F. Delong, Cambridge, MA (US)

Correspondence Address: WOLF GREENFIELD & SACKS, P.C., 600 ATLANTIC AVENUE, BOSTON, MA, 02210-2206, US

(21) Appl. No.: 12/196,587

(22) Filed: Aug. 22, 2008

Related U.S. Application Data
(60) Provisional application No. 60/965,915, filed on Aug. 23, 2007.

(51) Int. Cl. C12N 13/00; C12N 1/21; C12N 1/19; C12N 1/15; C12N 5/10

(52) U.S. Cl. 435/173.8; 435/252.3; 435/254.2; 435/254.11; 435/325; 435/173.1

(57) ABSTRACT

The invention relates to functional heterologous expression of proteorhodopsin photosystems in cells and the use of such proteorhodopsin photosystems in methods for increasing energy content of a cell, redirecting carbon flow in a cell, increasing a production of a carbon-based compound in a cell and increasing production of a metabolite in a cell.

### RELATED APPLICATIONS

This application claims the benefit under 35 U.S.C. § 119(e) of U.S. provisional application Ser. No. 60/965,915, filed Aug. 23, 2007, the disclosure of which is incorporated by reference herein in its entirety.

### GOVERNMENT INTEREST

This work was funded in part by the National Science Foundation under grant numbers MCB-0348001 and EF0424599. The government has certain rights in this invention.

### FIELD OF THE INVENTION

The invention relates to functional heterologous expression of proteorhodopsin photosystems in cells and the use of such proteorhodopsin photosystems in methods for increasing energy content of a cell, redirecting carbon flow in a cell, increasing a production of a carbon-based compound in a cell and increasing production of a metabolite in a cell.

### BACKGROUND OF THE INVENTION

Proteorhodopsins (PRs) are retinal-binding membrane proteins belonging to the rhodopsin family. Prokaryotic members of this family include photosensors (sensory rhodopsins), transmembrane proton pumps (bacteriorhodopsins, xanthorohodopsin, and PRs), and transmembrane chloride pumps (halorhodopsins). Originally discovered in Archaea, rhodopsins were later identified in Gammaproteobacteria of the SAR86 group during a cultivation-independent genomic survey. Dubbed proteorhodopsin, this photoprotein functions as a light-activated proton pump when expressed in Escherichia coli in the presence of exogenously added retinal (1). Since then, numerous molecular surveys have demonstrated that PR genes are ubiquitous in bacteria inhabiting the ocean's photic zone (2-9). An estimated 13% of bacteria in marine picoplankton populations, as well as a significant fraction of planktonic Euryarchaeota, contain a PR gene (4, 8). In a number of marine bacteria, retinal biosynthetic genes and PR are genetically linked, and their lateral transfer and retention appear to be relatively common events, indicating that the photosystem confers a significant fitness advantage (3, 4, 7, 10, 11). A recent report of light-stimulated growth in a PR-containing marine flavobacterium supports this hypothesis (11). Despite all of these observations however, the various specific functions and physiological roles of diverse marine microbial PRs remain to be fully described.

### SUMMARY OF THE INVENTION

To further characterize PR photosystem structure and function, we directly screened large-insert DNA libraries derived from marine picoplankton for visibly detectable PR-expressing phenotypes. Here we describe completely intact PR-based photosystems that can be functionally expressed in E. coli, without addition of exogenous photopigment (e.g., retinal or its precursors). Analyses of insertional mutants verified the functional annotation of each gene product in the photosystem biosynthetic pathway. We also show that light-activated, PR-catalyzed proton translocation, by the chemiosmotic potential it generates, activates photophosphorylation in E. coli.

According to one aspect of the invention, cell(s) including a functional heterologous proteorhodopsin photosystem are provided. Preferably the cell(s) are bacterial cell(s), archael cell(s), yeast cell(s), fungal cell(s) or mammalian cell(s). In some embodiments, the heterologous proteorhodopsin photosystem is introduced into the cell(s) as a set of linked genes on a fosmid or cosmid. In other embodiments, the heterologous proteorhodopsin photosystem comprises genes encoding a retinal biosynthesis pathway and proteorhodopsin (PR). Preferably the genes encoding the retinal biosynthesis pathway comprise crtE, crtB, crtI, crtY and blh.

According to a second aspect of the invention, methods for increasing energy content of a cell are provided. The methods include expressing in the cell a functional heterologous proteorhodopsin photosystem, and exposing the cell to light, whereby the functional heterologous proteorhodopsin photosystem synthesizes adenosine triphosphate (ATP) in the cell, thereby increasing the energy content of the cell. In some embodiments, the heterologous proteorhodopsin photosystem comprises genes encoding a retinal biosynthesis pathway and proteorhodopsin (PR). Preferably the genes encoding the retinal biosynthesis pathway comprise crtE, crtB, crtI, crtY and blh. In other embodiments, the cell is a bacterial cell, an archael cell, a yeast cell, a fungal cell or a mammalian cell. Preferably the cell is a bacterial cell; more preferably the bacterial cell is in a stationary phase.

According to a third aspect of the invention, methods for increasing production of a metabolite in a cell are provided. The methods include expressing in the cell a functional heterologous proteorhodopsin photosystem, culturing the cell to produce the metabolite, and exposing the cell to light, whereby the functional heterologous proteorhodopsin photosystem synthesizes adenosine triphosphate (ATP) in the cell, thereby increasing the energy of the cell available for production of the metabolite, wherein the production of the metabolite is increased. In some embodiments, the heterologous proteorhodopsin photosystem comprises genes encoding a retinal biosynthesis pathway and proteorhodopsin (PR). Preferably the genes encoding the retinal biosynthesis pathway comprise crtE, crtB, crtI, crtY and blh. In other embodiments, the cell is a bacterial cell, an archael cell, a yeast cell, a fungal cell or a mammalian cell. Preferably the cell is a bacterial cell; more preferably the bacterial cell is in a stationary phase.

According to a fourth aspect of the invention, methods to redirect carbon flow in a cell are provided. The methods include expressing in the cell a functional heterologous proteorhodopsin photosystem, and exposing the cell to light, whereby the functional heterologous proteorhodopsin photosystem synthesizes adenosine triphosphate (ATP) in the cell, thereby redirecting carbon compounds from production of ATP by the cell to production of other compounds by the cell. Preferably the other compounds are metabolites. In some embodiments, the heterologous proteorhodopsin photosystem comprises genes encoding a retinal biosynthesis pathway and proteorhodopsin (PR). Preferably the genes encoding the retinal biosynthesis pathway comprise crtE, crtB, crtI, crtY and blh. In other embodiments, the cell is a bacterial cell, an archael cell, a yeast cell, a fungal cell or a mammalian cell. Preferably the cell is a bacterial cell; more preferably the bacterial cell is in a stationary phase.

According to a fifth aspect of the invention, methods to increase a production of a carbon-based compound in a cell are provided. The methods include expressing in the cell a functional heterologous proteorhodopsin photosystem, and exposing the cell to light, whereby the functional heterologous proteorhodopsin photosystem synthesizes adenosine triphosphate (ATP) in the cell, thereby reducing the amount of carbon compounds needed for production of ATP by the cell, thereby making the carbon compounds available for increased production of the carbon-based compound by the cell. In some embodiments, the heterologous proteorhodopsin photosystem comprises genes encoding a retinal biosynthesis pathway and proteorhodopsin (PR). Preferably the genes encoding the retinal biosynthesis pathway comprise crtE, crtB, crtI, crtY and blh. In other embodiments, the cell is a bacterial cell, an archael cell, a yeast cell, a fungal cell or a mammalian cell. Preferably the cell is a bacterial cell; more preferably the bacterial cell is in a stationary phase.

These and other aspects of the invention are described further below.

### BRIEF DESCRIPTION OF THE DRAWINGS

FIG. 1. Genetic and phenotypic analysis of PR photosystem transposon mutants. (A) Schematic representation of the PR gene clusters identified in this work. Predicted transcription terminators in the clusters are indicated. (B) Color phenotype of intact cells of transposon-insertion mutants grown in liquid cultures with arabinose. (C) Retinal biosynthesis pathway. Names of genes encoding pathway enzymes are indicated. The genes that are present in E. coli are in parentheses. (D) HPLC profiles of wild-type and transposon mutant extracts. Detection wavelengths are indicated. Absorption spectrum of relevant peaks, including standards used for identification, are shown on the top for each panel.

FIG. 2. Proton-pumping assays. pH measurements are expressed as pH change with respect to the pH at time 0 for each sample. Gray boxes indicate dark periods.

FIG. 3. Light-activated, PR-enabled photophosphorylation in E. coli. (A) Diagram of the proposed mechanism of PR-dependent ATP synthesis. The effects of the inhibitors used are indicated. (B) Proton-pumping assays with HF10_{—}19P19 cells. pH measurements are expressed as the pH change respect to the pH at time 0 for each sample. CCCP, 25 μM; DCCD, 1 mM. Gray boxes indicate dark periods. (C) ATP assays with HF10_{—}19P19 cells. Results are expressed as the difference between the ATP level in the light and the ATP level in the dark, ΔATP, for each treatment.

FIG. 4. Light-driven differences in ATP and photophosphorylation with proteorhodopsin photosystems in different vectors in E. coli. (A) Graphical demonstration of ATP levels in proteorhodopsin-containing cells maintained in dark or light conditions for the indicated amount of time (pPRPS-MCL: proteorhodopsin photosystem genes expressed in plasmid pMCL200; pPRPS-FOS: proteorhodopsin photosystem genes expressed in plasmid pCC1FOS). (B) Graphical demonstration of increase in ATP levels in cultures maintained in the light, which is attributed to photophosphorylation from proteorhodopsin activity (pPRPS-MCL: proteorhodopsin photosystem genes expressed in plasmid pMCL200; pPRPS-FOS: proteorhodopsin photosystem genes expressed in plasmid pCC1FOS).

### DETAILED DESCRIPTION OF THE INVENTION

Proteorhodopsins (PRs) are retinal-containing proteins that catalyze light-activated proton efflux across the cell membrane. These photoproteins are known to be globally distributed in the ocean's photic zone, and they are found in a diverse array of Bacteria and Archaea. Recently, light-enhanced growth rates and yields have been reported in at least one PR-containing marine bacterium, but the physiological basis of light-activated growth stimulation has not yet been determined. To describe more fully PR photosystem genetics and biochemistry, we functionally surveyed a marine picoplankton large-insert genomic library for recombinant clones expressing PR photosystems in vivo. Our screening approach exploited transient increases in vector copy number that significantly enhanced the sensitivity of phenotypic detection. Two genetically distinct recombinants, initially identified by their orange pigmentation, expressed a small cluster of genes encoding a complete PR-based photosystem. Genetic and biochemical analyses of transposon mutants verified the function of gene products in the photopigment and opsin biosynthetic pathways. Heterologous expression of six genes, five encoding photopigment biosynthetic proteins and one encoding a PR, generated a fully functional PR photosystem that enabled photophosphorylation in recombinant Escherichia coli cells exposed to light. Our results demonstrate that a single genetic event can result in the acquisition of phototrophic capabilities in an otherwise chemoorganotrophic microorganism, and they explain in part the ubiquity of PR photosystems among diverse microbial taxa.

Methods for retrieval and amplification of proteorhodopsin genes from DNA samples of naturally occurring marine bacteria are described in US published patent application 2003/0104375. Certain proteorhodopsin proteins, variants and mutants, and their properties, as well as methods of making and using these are provided in US published patent application 2005/0095605 and references cited therein.

Proteorhodopsin genes and retinal biosynthesis pathway genes that constitute a functional proteorhodopsin photosystem can be cloned and introduced into cells using methods and vectors well known to the skilled person. The Examples of this application describe the use of a fosmid vector, but many other kinds of expression vectors are known in the art and could also be used.

As shown in the Examples, two approximately 40 kb pieces of genomic bacterial DNA in fosmid vectors each are sufficient to provide heterologous expression of a complete proteorhodopsin photosystem. In addition, as is shown in the Examples, only 6 genes are necessary for heterologous expression in E. coli. Therefore the invention described herein can be carried out using a natively arranged set of genes or can be carried out by expression of a smaller set of genes that provides heterologous expression of a complete proteorhodopsin photosystem. In the latter case, the genes may be, but need not be, cloned into and/or expressed from a single vector.

As used herein, a “vector” may be any of a number of nucleic acids into which a desired sequence or sequences may be inserted by restriction and ligation for transport between different genetic environments or for expression in a host cell. Vectors are typically composed of DNA although RNA vectors are also available. Vectors include, but are not limited to: plasmids, fosmids, phagemids, virus genomes and artificial chromosomes.

A cloning vector is one which is able to replicate autonomously or integrated in the genome in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase.

An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA). That heterologous DNA (RNA) is placed under operable control of transcriptional elements to permit the expression of the heterologous DNA in the host cell.

Aspects of the invention include strategies to optimize expression of a proteorhodopsin photosystem in a cell. In some embodiments optimizing expression of a proteorhodopsin photosystem means increasing the energy content of a cell, increasing production of a metabolite in a cell, increasing redirection of carbon flow in a cell, and/or increasing production of a carbon-based compound in a cell, relative to that achieved in the absence of an optimization strategy. One strategy to optimize expression levels of the proteorhodopsin photosystem is through selection of appropriate promoters and ribosome binding sites. In some embodiments this may include the selection and use of high-copy number plasmids, or low or medium-copy number plasmids, to produce an optimal level of expression of the proteorhodopsin photosystem in a given cell. The step of transcription termination can also be targeted for regulation of gene expression, for example through the introduction or elimination of structures such as stem-loops.

In some embodiments it may be advantageous to use a cell that is optimized for expression of the proteorhodopsin photosystem. For example it may be optimal to mutate one or more genes in a cell, prior to expression of the proteorhodopsin photosystem in the cell. In some embodiments, screening for mutations that lead to optimized expression of the proteorhodopsin photosystem may be conducted through a random mutagenesis screen, or through screening of known mutations.

Optimization of expression of the proteorhodopsin photosystem may involve in some embodiments modifying the genes within the proteorhodopsin photosystem prior to introducing the photosystem into a cell, such as through codon optimization. Codon usages for a variety of organisms can be accessed in the Codon Usage Database internet site.

Aspects of the invention relate to a heterologous proteorhodopsin photosystem comprising genes encoding a retinal biosynthesis pathway, and proteorhodopsin (PR). In some embodiments the genes encoding a retinal biosynthetic pathway include crtE, crtB, crtI, crtY and blh. While the sequences for two such photosystems are provided in SEQ ID NOs: 3 and 4, it should be appreciated that homologous photosystems from other organisms expressing crtE, crtB, crtI, crtY, blh and proteorhodopsin are also encompassed by the invention. In some embodiments, homologous photosystems will express variant or homologous forms of crtE, crtB, crtI, crtY, blh and/or proteorhodopsin. In general, homologs typically will share at least 75% nucleotide identity and/or at least 85% amino acid identity to the sequences of crtE, crtB, crtI, crtY, blh and proteorhodopsin nucleic acids and polypeptides, respectively. In some instances homologs will share at least 80%, 82%, 85%, 88%, 90%, 91%, 92%, 93%, 94%, 95%, 96%, 97%, 98%, 99%, or greater than 99% nucleotide identity and/or at least 80%, 82%, 85%, 88%, 90%, 91%, 92%, 93%, 94%, 95%, 96%, 97%, 98%, 99%, or greater than 99% amino acid identity. In some embodiments, the sequences of one or more of the genes comprising the photosystem may be generated synthetically. It should also be appreciated that the order of the genes within the photosystem (i.e. crtE, crtB, crtI, crtY, blh and proteorhodopsin) can be manipulated to optimize expression, as is well known in the art.

Heterologous expression of a complete proteorhodopsin photosystem was demonstrated in the Examples using E. coli. The complete proteorhodopsin photosystem also can be expressed in other bacterial cells, archael cells, yeast, fungi, mammalian cells, etc.

Expression of a heterologous proteorhodopsin photosystem is useful for a process to enhance yield or increase the potential of recombinant protein production or converting the light induced membrane potential into cellular signals, including modulation of gene expression. The biochemical energy derived from functional proteorhodopsin photosystem exposed to light could be harnessed to support a variety of cellular processes. For instance, the energy derived from proteorhodopsin photosystem exposed to light (e.g., increased adenosine 5′-triphosphate (ATP) production) could be used to enhance the production of secondary metabolites, or recombinant proteins in host cells, such as E. coli. Often, production of specific compounds in the biotechnology industry is limited, since their optimal expression or production occurs in the late stationary phase of growth, when energy reserves of the host cells are low. A proteorhodopsin photosystem expressed in such cells would provide an ample source of biochemical energy, by simple illumination. Proteorhodopsin photosystem-mediated light driven ATP production could enhance any variety of biosynthetic or physiological processes which require energy.

In addition, proteorhodopsin photosystem-mediated light driven ATP production would reduce the amount of carbon compounds used by cells to produce energy, since a light source would be sufficient to increase the cell's energy via the proteorhodopsin photosystem. Thus would allow carbon compounds that otherwise would be used for energy production to be used for other purposes by the cell, such as production of secondary metabolites, etc. This redirection of carbon flow from energy production to other uses permits increases in production of a compounds by a cell, particularly carbon-based compounds.

### EXAMPLES

### Materials and Methods

Fosmid Library. The HOT_{—}10m fosmid library screened in this work has been described previously (14). It contains DNA from a planktonic sample collected 10 m below the surface at the ALOHA station (22°45′ N, 158°W) of the Hawaii Ocean Time series (HOT) cloned into the copy-controlled pCC1FOS fosmid vector (Epicentre Biotechnologies, Madison, Wis.). The library host, E. coli EPI300 (Epicentre Biotechnologies), supports the copy-control option of pCC1FOS.
Screening for PR Expression. High-density colony macroarrays [12,280 clones of the HOT_{—}10m library (ref. 14)] were prepared on a Performa II filter (Genetix Ltd., Boston, Mass.) by using a Q-PixII robot (Genetix Ltd.). The filter was carefully laid over a 22-cm plate containing 250 ml of LB agar supplemented with L-arabinose (0.02%), the copy-up inducer, and all-trans-retinal (20 μM), and the plate was incubated at 37° C. for 24 h. The filters were used to facilitate the visual detection of color against the white background. Colonies were inspected visually for the appearance of orange or red color. Fosmid DNA from positive clones was retransformed into fresh E. coli EPI300 and rescreened as above to verify that the color was conferred by the fosmid. The end DNA sequence of the positive clones was obtained by using primers T7 and EpiFos5R as described previously (14).
In Vitro Transposition and Full Fosmid Sequencing. Fosmid clones to be characterized were submitted to random in vitro transposition by using the EZ-Tn5<kan-2> insertion kit (Epicentre Biotechnologies) according to the manufacturer's instructions. The transposition reaction was transformed by electroporation into EPI300 cells, and clones containing fosmids with Tn5 insertions were selected in LB chloramphenicol, kanamycin (12 μg/ml and 25 μg/ml, respectively). The color phenotype of individual Tn5-insertion clones was analyzed on LB plates containing chloramphenicol, kanamycin, and 0.02% L-arabinose as above. DNA sequencing off the Tn5 ends was performed by using KAN-2 FP-1 and KAN-2 RP-1 primers, a BigDye version 3.1 cycle sequencing kit, and ABI Prism 3700 DNA analyzer (Applied Biosystems, Forest City, Calif.). The complete DNA sequence was assembled by using Sequencher version 4.5 (Gene Codes Corporation, Ann Arbor, Mich.) and annotated with FGENESB (Softberry, Mount Kisco, N.Y.) and Artemis version 6 (The Wellcome Trust Sanger Institute, Cambridge, U.K.).
Carotenoid Extraction. Overnight cultures of the appropriate clones were diluted 1:100 into 50 ml of LB chloramphenicol (12 μg/ml) and incubated for 3 h at 37° C. with shaking (200 rpm). At that point, L-arabinose was added to a 0.02% final concentration, and cultures were incubated for 16 h. Cells were harvested by centrifugation and rinsed twice in salt solution. Cell pellets were kept frozen (−20° C.) in the dark. Frozen cells were extracted by sonication (5 min) in a cold 4:1 (vol/vol) mixture of acetone/methanol (OmniSolv; EMD Chemicals, Gibbstown, N.J.) with 0.1 mM butylated hydroxytoluene added as an antioxidant. Cells were pelleted by centrifugation, and the supernatant was removed and filtered through ashed silica gel (230-400 mesh; EMD Chemicals). Extracts were then concentrated by evaporation under dry N₂. All extraction steps were performed in darkness or low light to minimize carotenoid photooxidation.
HPLC Analysis. Chromatographic separation and analysis of carotenoids by high-performance liquid chromatography (HPLC) adapted a reverse-phase method from Barua and Olson (36). A 5-μm Zorbax-ODS C18 column (150×4.6 mm) (Agilent Technologies, Palo Alto, Calif.) was used at 30° C. in a column oven with a Waters (Milford, Mass.) 2795 separations module operated with MassLynx 4.0 software. Separation was achieved with a linear gradient at a flow rate of 0.8 ml/min: 100% solvent A to 100% B over 20 min followed by isocratic elution with B for an additional 20 min, where A=methanol/water (3:1 vol/vol) and B=methanol/dichloromethane (4:1 vol/vol). The column was equilibrated after each run with solvent A for 10 min. The detector was a Waters 996 photodiode array detector scanning wavelengths from 190 to 800 nm with a resolution of 1.2 nm and sampling rate of one spectrum per s. Carotenoids were identified by comparing absorbance spectra and retention times with authentic standards.
Proton-Pumping Experiments. Clones to be analyzed for proton-pumping activity were streaked on 15-cm LB agar plates containing 12 μg/ml chloramphenicol and 0.001% L-arabinose and incubated at 37° C. for 48 h. Cells were resuspended in 20 ml of salt solution (10 mM NaCl/10 mM MgCl₂/100 μM CaCl₂, pH7.0), rinsed twice, and adjusted to an A₆₀₀ of 0.5-0.7. Two milliliters of cell suspension was placed in an RPC-100 photosynthetic chamber (i-Works, Dover, N.H.) connected to a 22° C. circulating water bath. pH was measured by using a Beckman (Fullerton, Calif.) Φ360 pH meter equipped with a Futura microelectrode. Light was provided by a 160-watt halogen lamp placed 4 cm from the chamber. Irradiance within the chamber was 500-650 μmol Q m⁻² s⁻¹.
ATP Measurements. Cell suspensions were prepared as above. Three milliliters of cell suspension was placed in 5-ml screw-cap glass vials. The vials for the dark samples were wrapped in foil. Ten centimeters of water was used to minimize heat transfer to the samples. Irradiance under these conditions was 650 μmol Q m⁻² s⁻¹. ATP was measured by using a luciferase-based assay (BactTiter Glo, Promega, Madison, Wis.) as follows. At each time point, 5 aliquots (20 μl each) of every sample were dispensed into white 96-well assay plates [CoStar (Bethesda, Md.) 3917]. One hundred microliters of BactTiterGlo reagent was added per well, and luminescence was measured after 10 min using a Victor3 plate reader (PerkinElmer, Waltham, Mass.) with a 1-s integration time. An ATP standard curve was used to calculate the concentration of ATP in the samples. For inhibitor experiments, cell suspensions were incubated in the dark for 20 h in the presence of 1 mM DCCD or for 2 h in the presence of 25 μM CCCP or the ethanol vehicle. Succinate was added to a 0.2% final concentration to measure ATP synthesis from respiration in the dark.

### Results

### Screening a Fosmid Library for in Vivo PR Photosystem Expression.

When E. coli expresses a PR apoprotein from an inducible promoter on a high-copy number plasmid, the cells acquire a red or orange pigmentation in the presence of exogenous all-trans retinal (1, 12). Retinal addition is required because E. coli lacks the ability to biosynthesize retinal or its precursor, β-carotene. Based on these observations, we screened for PR-containing clones on retinal-containing LB agar plating medium, which we expected would display an orange to red phenotype under these conditions. To enhance assay sensitivity, we used the copy-control system present in our fosmid vector that allowed a controlled transition from one copy per cell to multiple (up to 100) vector copies upon addition of the inducer L-arabinose (13).

A fosmid library prepared from ocean surface water picoplankton containing 12,280 clones (≈440 Mb of cloned DNA) (14) was screened by using the above approach. Three orange colonies were identified as potential PR-expressing clones on the LB-retinal-L-arabinose agar plates. All three showed no pigmentation in the absence of the high-copy number inducer. Unexpectedly, these clones also displayed an orange phenotype in the absence of L-retinal when induced to high copy number. The sequence of one clone, HF10_{—}19P19, revealed the presence of a PR gene near the fosmid vector junction (see below). Because the clones exhibited orange pigmentation in the absence of exogenous retinal, we expected that they must also be expressing retinal biosynthetic genes. Two clones, HF10_{—}25F10 and HF10_{—}19P19, were analyzed further for PR photosystem gene expression and function.

### Genomic Analyses of Candidate PR-Photosystem Expressing Clones.

The full DNA sequence of the two putative PR photosystem-containing fosmids was obtained by sequencing a collection of transposon-insertion clones. The approach facilitated rapid DNA sequencing while simultaneously providing a set of precisely located insertion mutants for phenotypic analysis of specific gene functions (15).

These sequences are deposited in GenBank under accession numbers EF100190 and EF100191 and are provided herewith as SEQ ID NOs: 1 and 2, respectively. The GenBank entries are annotated with individual gene sequences and polypeptide sequences encoded thereby. The HF10_{—}19P19 photosystem (coding strand) is provided as SEQ ID NO:3. The HF10_{—}25F10 photosystem (coding strand) is provided as SEQ ID NO:4. Primer sequences used to amplify the HF10_{—}19P19 photosystem are provided in SEQ ID NOs:5 and 6.

Both PR photosystem-containing clones were derived from Alphaproteobacteria based on ORF content similarity to homologues in the National Center for Biotechnology Information nonredundant protein database [Tables 1 and 2]. The clones exhibited highest identity to other PR-containing BAC clones from Alphaproteobacteria from the Mediterranean and Red Seas (8). This similarity was evident across the entire cloned insert, although some large-scale rearrangements were apparent. The HF10_{—}19P19 PR-inferred protein sequence was most similar to a homologue from another environmental BAC, MedeBAC66A03 (67% identity, 83% similarity). The MedeBAC66A03 PR was previously reported to exhibit fast photocycle kinetics and light-activated proton translocation when expressed in E. coli in the presence of exogenous retinal (8). Clone HF10_{—}25F10 PR was most similar in inferred protein sequence to another BAC clone, RED17H08 PR (93% identity, 97% similarity) and was very similar to MedeBAC66A03 as well (62% identity, 78% similarity). Both of the PR genes analyzed here encoded proteins with a glutamine residue at position 105, a characteristic of blue light-absorbing PRs (5) and consistent with the orange pigmentation observed in clones expressing them.

TABLE 1
List of predicted genes on fosmid HF10_19P19 (41802 bp).

| | | | | | Best BLASTP bit | | | |
|---|---|---|---|---|---|---|---|---|
| N | Start | End | Strand | Description | GenBank gi | −log(e) | % identity | Texonomic afflication |
| 1 | 3 | 330 | − | Flagellar protein MotB | 91218602 | 55 | 100 | Psychroflexus torquis (Bacteriodetas/ |
| | | | | | | | | Chlorobi) |
| 2 | 386 | 1147 | − | Flagellar protein MotA | 68164501 | >100 | 78 | uncultured marine bacterium BAC17H8 |
| 3 | 1342 | 1914 | + | Hypothetical protein | 68164500 | 38 | 51 | uncultured marine bacterium BAC17H8 |
| 4 | 1911 | 2093 | − | Hypothetical protein | — | | | |
| 5 | 2411 | 3925 | + | DNA-directed RNA polymerase | 68164498 | >100 | 49 | uncultured marine bacterium BAC17H8 |
| | | | | specalized sigma subunit, sigma54 | | | | |
| 6 | 4042 | 4221 | − | Hypothetical protein | — | | | |
| 7 | 4340 | 5686 | − | Peptidase | 68164492 | 54 | 34 | uncultured marine bacterium BAC17H8 |
| 8 | 5696 | 6073 | − | Hypothetical protein | 68164491 | 10 | 32 | uncultured marine bacterium BAC17H8 |
| 9 | 6272 | 6681 | + | Membrane protein | 87198088 | 12 | 33 | Novosphingobium aromaticivorans |
| | | | | | | | | (Alpha Proteobacteria) |
| 10 | 6673 | 7110 | + | Membrane protein | 68164489 | 21 | 42 | uncultured marine bacterium BAC17H8 |
| 11 | 7103 | 7441 | + | Hypothetical protein | 68164488 | 14 | 39 | uncultured marine bacterium BAC17H8 |
| 12 | 7417 | 7746 | − | Hypothetical protein | — | | | |
| 13 | 7763 | 8212 | − | Hypothetical protein | 68164493 | 5 | 28 | uncultured marine bacterium BAC17H8 |
| 14 | 8533 | 8877 | + | Hypothetical protein | — | | | |
| 15 | 8911 | 9558 | − | Hypothetical protein | 67906762 | 82 | 64 | uncultured marine bacterium |
| | | | | | | | | MedeBAC46A06 |
| 16 | 9716 | 10858 | + | Aminotransferase | 85704324 | >100 | 60 | Roseovarius sp (Alpha Proteobacteria) |
| 17 | 11065 | 11463 | − | Phosphoribosyl-AMP cyclohydrase | 78700320 | 39 | 63 | Alkalimnicola ehrichel (Gamma |
| | | | | | | | | Proteobacteria) |
| 18 | 11598 | 11765 | + | Hypothetical protein | — | | | |
| 19 | 11774 | 12367 | − | Methyltransferase type 12 | 94310012 | 21 | 42 | Loktanetla vestfoldensis (Alpha |
| | | | | | | | | Proteobacteria) |
| 20 | 12364 | 13230 | − | Sulfate/tungstate uptake family ABC | 56678404 | 70 | 52 | Ralstonia metallidurans (Beta |
| | | | | | | | | Proteobacteria) |
| | | | | transporter, substrate-binding protein | | | | |
| 21 | 13372 | 13782 | + | Transcription regulator, ModE family | 87200527 | 6 | 36 | Silicbacter pomeroyi (Alpha |
| | | | | | | | | Proteobacteria) |
| 22 | 13832 | 14119 | + | Hypothetical protein | 67906748 | 24 | 56 | Novosphingobium aromaticivorans (Alpha |
| | | | | | | | | Proteobacteria) |
| 23 | 14138 | 14590 | + | Hypothetical protein | 67906749 | 22 | 45 | uncultured marine bacterium |
| | | | | | | | | MedeBAC46A08 |
| 24 | 14630 | 14875 | + | Hypothetical protein | 83950410 | 5 | 48 | Roseovarius nubinhibens (Alpha |
| | | | | | | | | Proteobacteria) |
| 25 | 14875 | 15975 | + | ATPase | 67906750 | >100 | 71 | uncultured marine bacterium |
| | | | | | | | | MedeBAC46A06 |
| 26 | 15972 | 16760 | + | Hypothetical protein | 67906751 | 61 | 49 | uncultured marine bacterium |
| | | | | | | | | MedeBAC46A06 |
| 27 | 16757 | 17272 | + | Ribosomal protein L7/L12 | 67906752 | 47 | 57 | uncultured marine bacterium |
| | | | | | | | | MedeBAC46A06 |
| 28 | 17241 | 18248 | + | Hypothetical protein | 67906753 | >100 | 57 | uncultured marine bacterium |
| | | | | | | | | MedeBAC46A06 |
| 29 | 18312 | 20321 | − | Iron-sulfer cluster-binding protein | 67906754 | >100 | 59 | uncultured marine bacterium |
| | | | | | | | | MedeBAC46A06 |
| 30 | 20469 | 21077 | + | Hypothetical protein | 67906755 | 52 | 55 | uncultured marine bacterium |
| | | | | | | | | MedeBAC46A06 |
| 31 | 21105 | 21848 | + | Hypothetical protein | 67906756 | 36 | 40 | uncultured marine bacterium |
| | | | | | | | | MedeBAC46A06 |
| 32 | 22048 | 22671 | + | Cytoplasmic chaperone TorD | 67906757 | 64 | 61 | uncultured marine bacterium |
| | | | | | | | | MedeBAC46A06 |
| 33 | 22747 | 22941 | + | Hypothetical protein | 89092830 | 7 | 49 | Oceanospiritum sp. (Gamma |
| | | | | | | | | Proteobacteria) |
| 34 | 22993 | 25884 | + | Anaerobic dehydrogenase | 67906758 | >100 | 89 | uncultured marine bacterium |
| | | | | alpha subunit | | | | MedeBAC46A08 |
| 35 | 25897 | 26490 | + | Anaerobic dehydrogenase, | 83941749 | >100 | 89 | Subtobacter sp. (Alpha Proteobacteria) |
| | | | | iron-sulfur subunit | | | | |
| 36 | 26937 | 27919 | + | Anaerobic dehydrogenase | 67906759 | >100 | 72 | uncultured marine bacterium |
| | | | | gamma subunit | | | | MedeBAC46A06 |
| 37 | 28072 | 29379 | + | WD domain/cytochrome C family | 67906760 | >100 | 58 | uncultured marine bacterium |
| | | | | protine | | | | MedeBAC46A08 |
| 38 | 29398 | 29520 | + | Hypothetical protein | — | | | |
| 39 | 29571 | 30272 | + | Trypsin-like serne protease | 67906761 | 58 | 54 | uncultured marine bacterium |
| | | | | | | | | MedeBAC46A98 |
| 40 | 30364 | 31260 | + | Molybdenum cofactor biosynthesis | 67527057 | >100 | 70 | uncultured marine bacterium 68A93 |
| | | | | protein | | | | |
| 41 | 31445 | 31984 | + | Molybdotenin guanine dinucleotide | 71847033 | 43 | 56 | Dechlioromonas aromatica (Beta |
| | | | | biosynthesis protein | | | | Proteobacteria) |
| 42 | 31993 | 38225 | + | Molybdopterin biosynthesis protein | 67527055 | 90 | 44 | uncultured marine bacterium 68A93 |
| 43 | 33225 | 33476 | + | Molybdopterin-converting factor, | 23016726 | 22 | 60 | Magnetospirillum magnetotacitcum MS-1 |
| | | | | small subunit | | | | |
| 44 | 33481 | 33960 | + | Molybdopterin-converting factor | 27382584 | 42 | 52 | Bradyrhizobium japonicum (Alpha |
| | | | | large subunit | | | | Proteobacteria) |
| 45 | 34208 | 35227 | − | isopertend diphosphate delta- | 68164580 | 86 | 54 | uncultured marine bacterium 66A93 |
| | | | | isomerase Idi | | | | |
| 46 | 35224 | 36084 | − | 15,15′-beta-carotene dioxygenase Bth | 67527050 | 50 | 40 | uncultured marine bacterium BAC17H8 |
| 47 | 36127 | 37257 | − | Lycopene cycase CrtY | 68164582 | 73 | 41 | uncultured marine bacterium 66A02 |
| 48 | 37254 | 38237 | − | Phytoene synthase CrtB | 67527048 | 68 | 47 | uncultured marine bacterium BAC17H8 |
| 49 | 38218 | 39765 | − | Phytoene dehydrogenase CrtI | 67906784 | >100 | 71 | uncultured marine bacterium |
| | | | | | | | | MedeBAC66A02 |
| 50 | 39855 | 40871 | − | Geranylgeranyl pyrophosphate | 67527046 | 86 | 51 | uncultured marine bacterium 66A02 |
| | | | | synthetase CrtE | | | | |
| 51 | 40993 | 41766 | − | Proteorhodopsin | 67527045 | 97 | 67 | uncultured marine bacterium 66A03 |

TABLE 2
List of predicted genes on fosmid HF10_25F10 (40220 bp).

| | | | | | Best BLASTP bit | | | |
|---|---|---|---|---|---|---|---|---|
| N | Start | End | Strand | Description | GenBank gi | −log(e) | % Identity | Taxonomic affiliation |
| 1 | 96 | 365 | + | Hypothetical protein | 67906766 | 44 | 98 | uncultured bacterium MedeBAC46A06 |
| 2 | 460 | 2724 | + | Hypothetical protein | 67906767 | >100 | 94 | uncultured bacterium MedeBAC46A06 |
| 3 | 2732 | 4024 | + | Histidinol dehydrogenase | 67906768 | >100 | 89 | uncultured bacterium MedeBAC46A06 |
| 4 | 4069 | 4566 | + | Sulfopyruvate decarboxylase | 67906769 | >100 | 55 | uncultured bacterium MedeBAC46A06 |
| | | | | subunit alpha | | | | |
| 5 | 4571 | 5125 | + | Sulfopyruvate decarboxylase | 67906770 | >100 | 83 | uncultured bacterium MedeBAC46A06 |
| | | | | subunit ComD | | | | |
| 6 | 5133 | 5303 | + | Gluconate 5-dehydrogenase | 67906771 | 93 | 69 | uncultured bacterium MedeBAC46A06 |
| 7 | 5800 | 5863 | + | Sorbitol dehydrogenase | 67906772 | 82 | 74 | uncultured bacterium MedeBAC46A06 |
| 8 | 6876 | 7740 | + | Deoxyribodipyrmidine photolyase | 67906774 | 48 | 69 | uncultured bacterium MedeBAC46A06 |
| 9 | 7733 | 8182 | + | Unknown | 68164595 | 0 | 28 | uncultured bacterium BAC17H8 |
| 10 | 8269 | 9801 | + | Propionyl-CoA carboxylase beta | 68164594 | >100 | 87 | uncultured bacterium BAC17H8 |
| | | | | subunit | | | | |
| 11 | 9844 | 11820 | + | Propionyl-CoA carboxylase alpha | 67906776 | >100 | 93 | uncultured bacterium MedeBAC46A06 |
| | | | | subunit | | | | |
| 12 | 11817 | 12989 | − | Amidohydrolase family protein | 86138204 | >100 | 95 | Roseobacter sp. (Alpha Protecbacteria) |
| 13 | 12968 | 14605 | − | Oxidoreductase | 67906777 | >100 | 80 | uncultured bacterium MedeBAC46A06 |
| 14 | 14726 | 15472 | + | Membrane protein | 68164591 | >100 | 84 | uncultured bacterium BAC17H8 |
| 15 | 15481 | 16095 | + | 3-Methyladenine DNA glycosylase | 67906779 | >100 | 83 | uncultured bacterium MedeBAC46A06 |
| 16 | 16112 | 16504 | − | Unknown | 68164589 | >100 | 66 | uncultured bacterium BAC17H8 |
| 17 | 16416 | 16778 | − | Alpha-amylase | 41411249 | >100 | 71 | Butylvibrio fibrosolvens (Clostridia) |
| 18 | 16879 | 17697 | − | Hypothetical protein | 67906781 | >100 | 79 | uncultured bacterium MedeBAC46A06 |
| 19 | 18271 | 19047 | + | Proteorhodopsin | 68164586 | 98 | 70 | uncultured bacterium BAC17H8 |
| 20 | 19146 | 20171 | + | Geranylgeranyl pyrophosphate | 67906783 | 69 | 87 | uncultured bacterium MedeBAC46A06 |
| | | | | synthase CrtE | | | | |
| 21 | 20182 | 21756 | + | Phytoene dehydrogenase CrtI | 67906784 | >100 | 87 | uncultured bacterium MedeBAC46A06 |
| 22 | 21775 | 22692 | + | Phytoene synthase CrtB | 67906785 | >100 | 85 | uncultured bacterium MedeBAC46A06 |
| 23 | 22643 | 23791 | + | Lycopene cyclase CrtY | 67906786 | >100 | 93 | uncultured bacterium MedeBAC46A06 |
| 24 | 23818 | 24648 | + | 15,15′-beta-carotene dioxygenase Bth | 67906787 | >100 | 89 | uncultured bacterium MedeBAC46A06 |
| 25 | 24665 | 25740 | + | Isopentenyl-diphosphate delta | 67906788 | 90 | 88 | uncultured bacterium MedeBAC46A06 |
| | | | | isomerase isomerase Idi | | | | |
| 26 | 25747 | 26499 | − | Hydroxyacylglutathione hydrolase | 68164579 | >100 | 91 | uncultured bacterium BAC17H8 |
| 27 | 26504 | 26926 | − | Lactoylglutathione lyase | 68164578 | 16 | 45 | uncultured bacterium BAC17H8 |
| 28 | 27059 | 28051 | − | 2-Dehydropantoate 2-reductase | 67906791 | >100 | 74 | uncultured bacterium MedeBAC46A06 |
| 29 | 28089 | 29588 | − | Acyl-CoA synthetase | 67906792 | >100 | 93 | uncultured bacterium MedeBAC46A06 |
| 30 | 29986 | 30960 | + | Conserved hypothetical protein | 67906793 | >100 | 77 | uncultured bacterium MedeBAC46A06 |
| 31 | 31071 | 31601 | + | Hypothetical protein | 67906794 | >100 | 68 | uncultured bacterium MedeBAC46A06 |
| 32 | 31632 | 33167 | + | Conserved hypothetical protein | 67906795 | 65 | 92 | uncultured bacterium MedeBAC46A06 |
| 33 | 33280 | 33483 | + | Conserved hypothetical protein | 67906797 | >100 | 90 | uncultured bacterium MedeBAC46A06 |
| 34 | 33571 | 34506 | + | Ketopantoate reductase | 67906798 | 82 | 90 | uncultured bacterium MedeBAC46A06 |
| 35 | 34529 | 35332 | + | Aldolase class II | 68164572 | >100 | 83 | uncultured bacterium BAC17H8 |
| 36 | 35333 | 36496 | − | Aminotransferase | 67906801 | 0 | 33 | uncultured bacterium MedeBAC46A06 |
| 37 | 36609 | 37511 | − | Transcriptional regulator | 67906803 | 91 | 93 | uncultured bacterium MedeBAC46A06 |
| 38 | 37636 | 38025 | + | Alpha subunit of protocatechuate | 68164568 | >100 | 82 | uncultured bacterium BAC17H8 |
| | | | | 4,5-dioxygenase | | | | |
| 39 | 38027 | 38866 | + | Beta subunit of protocatechuate | 68164567 | >100 | 74 | uncultured bacterium BAC17H8 |
| | | | | 4,5-dioxygenase | | | | |
| 40 | 38901 | 39626 | + | Acyl transferase | 68164566 | >100 | 86 | uncultured bacterium BAC17H8 |
| 41 | 39910 | 40116 | − | Hypothetical protein | 18161831 | 45 | 57 | Pyrobaculum aerophium (Crenarchaeota) |

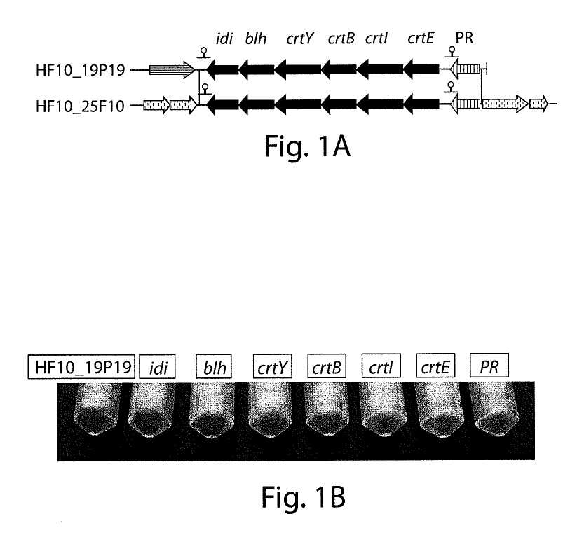

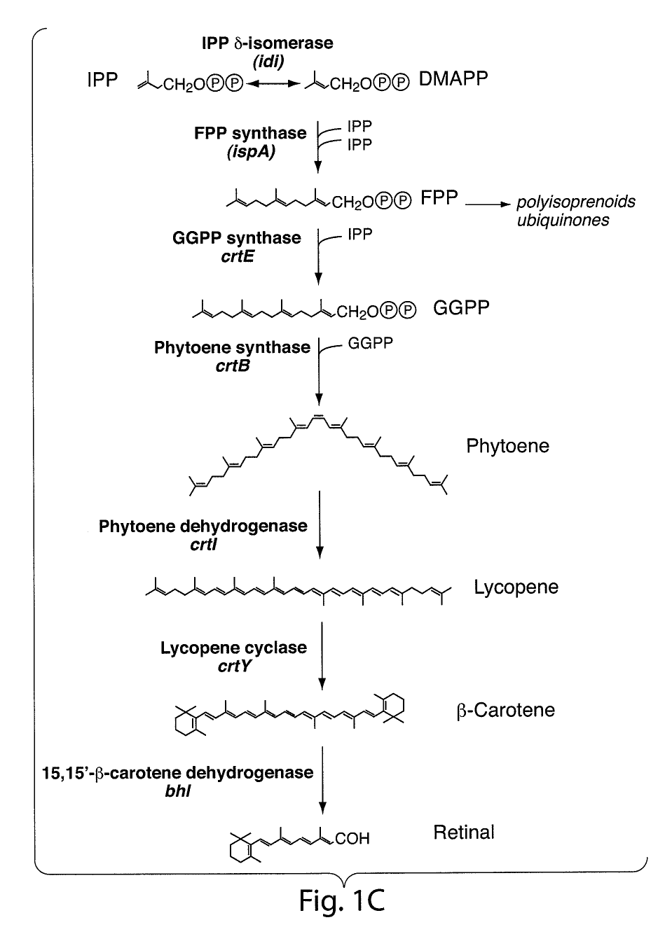

Adjacent to the PR gene in both clones was a predicted six-gene operon encoding putative enzymes involved in β-carotene and retinal biosynthesis (FIG. 1A). A similar arrangement was reported in MedeBAC66A03 and RED17H08 (8) and more recently in a wide variety of diverse marine bacterial groups (7). The genes encoded on these operons include crtE [putative geranylgeranyl pyrophosphate (GGPP) synthase], crtI (phytoene dehydrogenase), crtB (phytoene synthase), crtY (lycopene cyclase), blh (15,15′-β-carotene dioxygenase), and idi [isopentenyl diphosphate (IPP) δ-isomerase]. The putative role of these proteins in the retinal biosynthetic pathway (for review, see ref. 16) is indicated in FIG. 1C. The first reactions in the pathway are catalyzed by the IPP δ-isomerase and farnesyl diphosphate (FPP) synthase. Both enzymes are part of the isoprenoid and ubiquinone metabolic pathways and are present in E. coli. crtE, crtB, crtI, and crtY appear to encode all of the enzymes necessary to synthesize β-carotene from FPP. The blh gene found in MedeBAC66A03 was previously shown to encode a 15,15′-β-carotene dioxygenase that cleaves β-carotene, producing two molecules of all-trans-retinal (8).

Apart from the PR and putative β-carotene and retinal biosynthesis operon, no other genes were shared between the two PR-containing fosmids. With the exception of a gene encoding a putative deoxyribodipyrimidine photolyase in HF10_{—}25F10, no other genes flanking the PR photosystem had an obvious, light-related function (Tables 1 and 2).

### Genetic and Phenotypic Analysis of the PR Photosystem.

To obtain direct evidence for the functional annotations of putative retinal biosynthesis genes, we analyzed different transposon mutant phenotypes that carried an insertion in predicted PR photosystem genes. The cell pigmentation and HPLC pigment analyses in selected mutants are shown in FIGS. 1B and D, respectively. HF10_{—}19P19 cells carrying the intact vector were orange when grown in the presence of arabinose, consistent with expression of a blue-adapted retinal-PR complex. HPLC analysis revealed the presence of retinal in cell extracts, demonstrating that clone HF10_{—}19P19 contained all genes required for retinal biosynthesis in E. coli. Neither lycopene nor β-carotene was observed in the intact clone extracts, indicating that there was little if any accumulation of pigment intermediates (FIG. 1D). Cells containing transposon insertions in the idi gene were also orange and contained retinal. The lack of phenotype in this mutant can be attributed to the presence of the endogenous idi gene in E. coli (17).

As expected, transposon insertion mutants disrupted in the PR gene itself were devoid of orange pigmentation, and HPLC analysis showed a low but detectable level of retinal in these extracts (data not shown). It is unclear at present whether the low levels of retinal were due to polar effects caused by the transposon insertion in downstream expression or whether they result from pathway inhibition due to product accumulation.

Transposon-insertion mutants in crtE, crtB, and crtI showed no pigmentation, as expected for this biosynthetic pathway if it is interrupted before lycopene formation, the first colored product in the pathway. crtY-insertion mutants, however, were pink, suggesting that they were accumulating lycopene. Pigment analysis verified that crtY-insertion mutant extracts contained lycopene but not retinal or β-carotene (FIGS. 1B and D). Finally, blh-insertion mutants had a yellow phenotype, and HPLC analysis showed that these cells lacked detectable retinal but instead accumulated β-carotene. This finding demonstrates that the blh gene in HF10_{—}19P19 encodes a 15,15′-β-carotene dioxygenase, similar to a homologue recently described by Sabehi et al. (8). Transposon insertions in all other predicted genes outside the PR cluster had no visibly obvious phenotype. Identical pigmentation phenotypes were observed with insertions in the corresponding genes of the other PR photosystem clone, HF10_{—}25F10. Taken together, these results strongly support the functional assignments of PR-associated retinal biosynthetic pathway genes and demonstrate that they are necessary and sufficient to induce retinal biosynthesis in E. coli. Light activated proton-translocation.

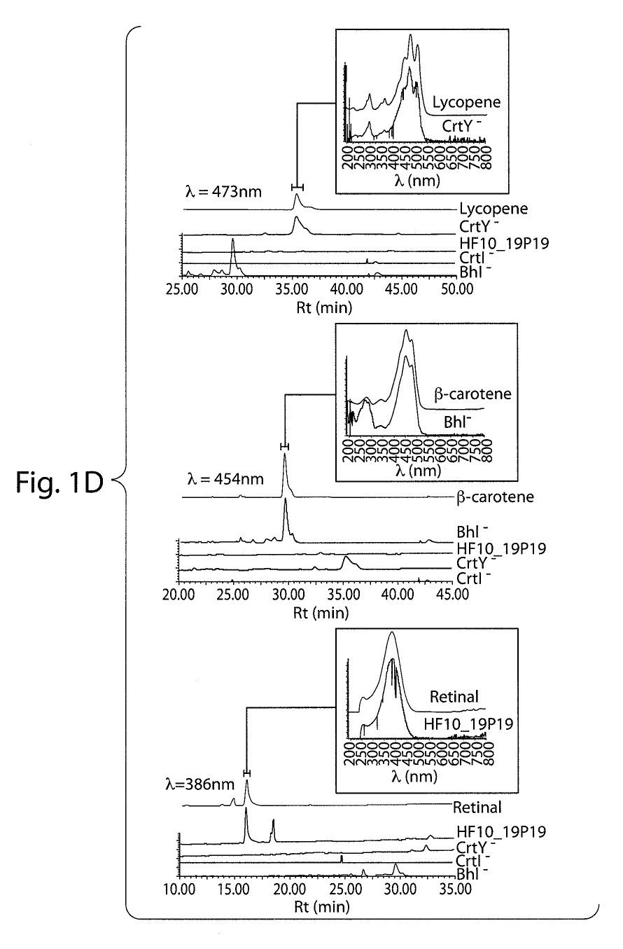

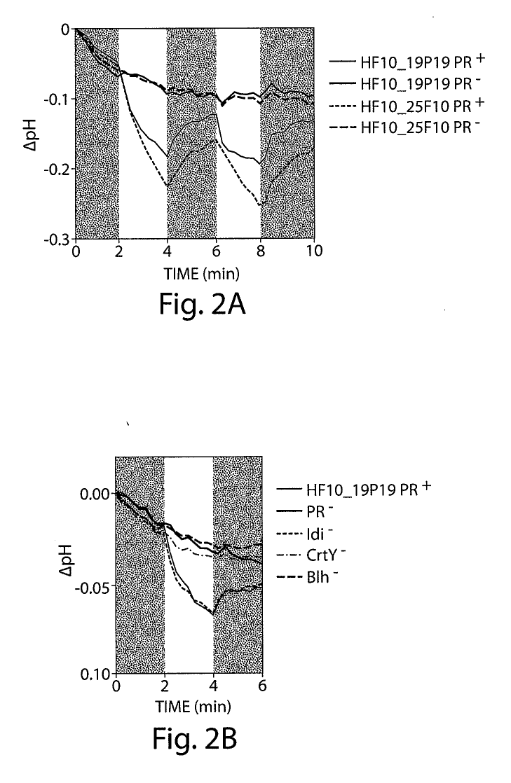

We assayed both HF10_{—}19P19 and HF10_{—}25F10 grown under high-copy number conditions for light-activated proton-translocating activity. Light-dependent decreases in pH were observed in PR⁺ clones but not in mutants containing a transposon insertion in the PR gene (PR⁻) (FIG. 2A). In addition, no light-dependent proton-translocating activity was observed in insertion mutants unable to synthesize retinal (CrtY⁻ or Blh⁻). In contrast, Idi⁻ mutants had normal proton-pumping activity, confirming that this gene was not required under our growth conditions (FIG. 2B). These results demonstrate that both fosmids independently expressed a functional PR with light-activated proton-translocating activity.

PR Driven Proton Translocation Catalyzes Photophosphorylation in E. coli.

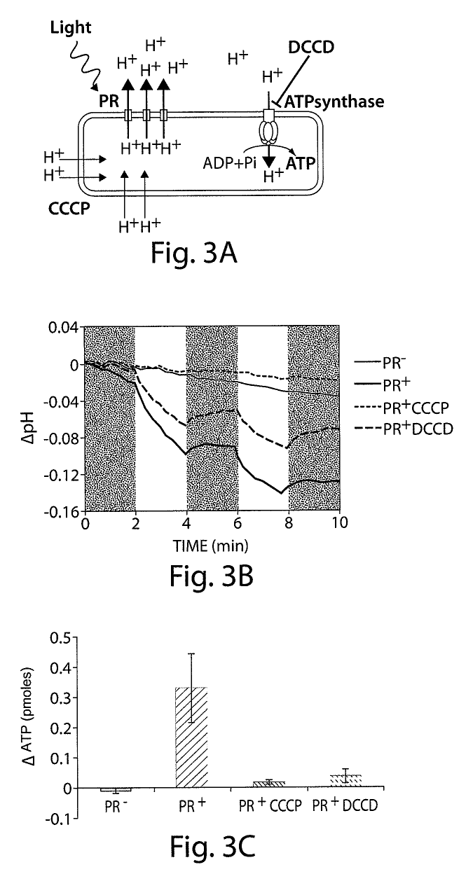

Analogous to earlier studies of haloarchaeal bacteriorhodopsins (18, 19), it was previously postulated that light-activated, PR-induced proton motive force could drive ATP synthesis as protons reenter the cell through the ATP synthase complex (FIG. 3A) (1, 12). This hypothesis was not previously tested, however, in either native or heterologously expressed PR-based photosystems. To this end, we measured light-induced changes in ATP levels in the PR-photosystem-containing clones and PR⁻ mutant derivatives by using a luciferase-based assay. The assay measures total ATP, and so we expected to observe increases in ATP concentration only if PR-driven ATP biosynthesis exceeded endogenous turnover rates, under our experimental conditions. Control pH measurements indicated that PR⁺ cells used in the ATP assay were indeed capable of light-activated proton translocation (FIG. 3B). ATP measurements performed after 5 min of illumination showed significant light-induced increases in cellular ATP levels in the PR⁺ clone but not in a PR⁻ mutant (FIG. 3C). The 0.3-pmol increase in ATP observed in the PR⁺ cells exposed to light (FIG. 3C) represents a 29% increase over identical cell preparations maintained in the dark, which corresponds to a net gain after 5 min of illumination of ≈2.2×10⁵ molecules of ATP per colony-forming unit (or viable cell) assayed. For comparison, oxidative phosphorylation, measured by ATP increases observed 5 min after the addition of 0.2% succinate to PR⁺ cells in the dark, resulted in a net gain of 9×10⁵ ATP molecules per live cell (data not shown).

Similar light-activated, PR-catalyzed photophosphorylation was also observed in cells containing the HF10_{—}25F10 fosmid. Although the PR photosystem of this clone is similar to that of HF10_{—}19P19, all of the genes flanking the two different photosystem gene suites are completely different and derive from different chromosomal contexts. Because the PR and retinal biosynthetic genes are the only shared genes on both clones, the results strongly suggest that these specific PR photosystem genes are both necessary and sufficient to drive photophosphorylation in E. coli cells.

To characterize more fully the light-driven photophosphorylation observed in PR⁺ E. coli cells, we tested the effects of carbonylcyanide m-chlorophenylhydrazone (CCCP), an uncoupler, and N,N′-dicyclohexylcarbodiimide (DCCD), a covalent inhibitor of H⁺-ATP synthase, on light-driven ATP synthesis (FIG. 3). We used concentrations that inhibited aerobic growth of E. coli on succinate, an oxidative phosphorylation process requiring both proton-motive force and ATP synthase activity (20). Addition of CCCP, which permeabilizes the cell membrane to H⁺, completely abolished the light-driven decrease in pH and subsequently photophosphorylation. This result demonstrates that both processes depend on the establishment of an H⁺ electrochemical gradient. In contrast, addition of the H⁺-ATP synthase inhibitor DCCD did not affect external pH changes resulting from PR-catalyzed proton translocation, but it completely abolished photophosphorylation. This result indicates that H⁺-ATP synthase is indeed responsible for the light-activated ATP increases we observed in PR⁺ cells.

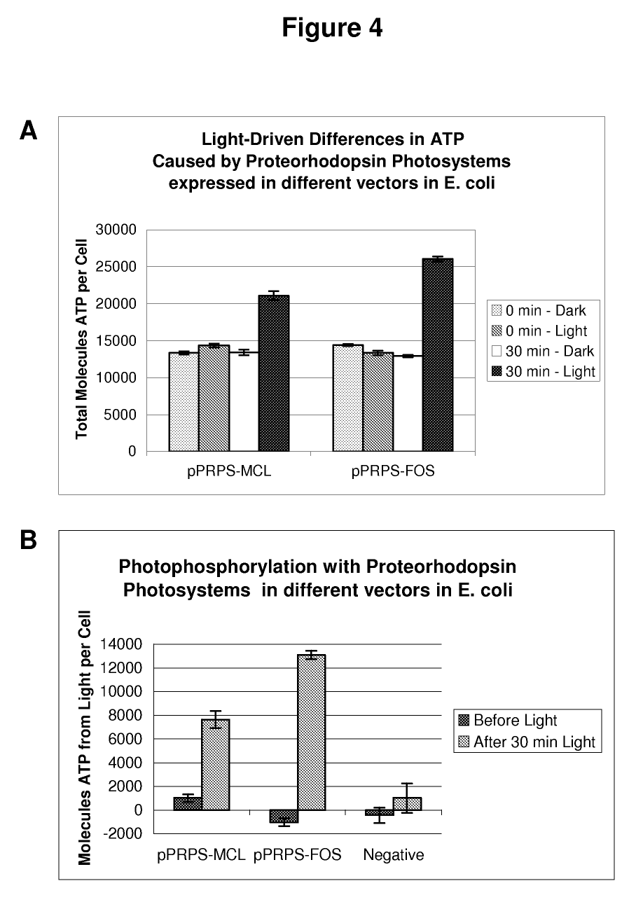

To further demonstrate the functionality of the proteorhodopsin photosystem, it was subcloned into the vector pMCL200 (37). SEQ ID NO:7 provides the proteorhodopsin photosystem sequence that was subcloned into pMCL200. Light-driven differences in ATP and photophosphorylation were compared between cells expressing the proteorhodopsin photosystem cloned in pMCL200 (pPRPS-MCL) and cells expressing the proteorhodopsin photosystem cloned in pCC1FOS (pPRPS-FOS), demonstrating that the photosystem was functional in both vectors (FIG. 4). ATP levels were determined as described above.

### Discussion

The results presented here demonstrate the utility of functionally screening large-insert DNA “metagenomic” libraries for new phenotypes and activities directly and without subcloning, an approach pioneered by soil microbiologists (15, 21, 22). Although large-insert libraries increase the probability of capturing complete metabolic pathways in a single clone, their low copy number decreases the sensitivity of detecting heterologous gene expression. We show here that increasing fosmid copy number (13) can significantly enhance detectable levels of recombinant gene expression and therefore increases the detection rate of desired phenotypes in metagenomic libraries. The PR photosystem recombinants we characterized could be detected visually by pigment production and exhibited light-dependent proton translocation and subsequent photophosphorylation, only when the fosmid vector was induced to high copy number. The approach was not completely effective in detecting all targeted genotypes however. Even under “copy-up” conditions, we were unable to detect all PR-containing clones known to exist in our library (4, 7). Despite the limitations, this approach for functional screening of microbial community genomic libraries is useful for identifying specific activities or phenotypes, even in the absence of sequence information. Additionally, this approach provides useful material for downstream genetic and biochemical characterization and for testing hypotheses derived from bioinformatic analyses.

Genetic and biochemical characterization of PR photosystem-containing clones reported here provided direct evidence that only six genes are required to enable light-activated proton translocation and photophosphorylation fully in a heterologous host. Sabehi et al. (8) demonstrated previously that coexpression of marine bacterial blh with PR, in the presence of the β-carotene biosynthetic genes from Erwinia herbicola, led to β-carotene cleavage and subsequent formation of retinal-bound PR. We show here that a set of six genetically linked genes known to be found in a wide variety of different marine bacterial taxa (7, 8, 11) are both necessary and sufficient for the complete synthesis and assembly of a fully functional PR photoprotein in E. coli. These heterologously expressed marine bacterial photosystems exhibited light-dependent proton translocation activity in the absence of exogenously added retinal or β-carotene. One gene in the PR photosystem cluster was dispensable under our conditions: the idi gene that encodes IPP δ-isomerase, an activity already present in E. coli (17), as is the FPP synthase, catalyzing the next two steps in the pathway (23). The presence of the idi gene in the cluster likely enables retinal production in the native organism because isomerization of IPP to dimethylallyl pyrophosphate can be a rate-limiting step in β-carotene biosynthesis (24, 25).

It was previously postulated that light-activated proton translocation catalyzed by PR elevates the proton-motive force, thereby driving ATP synthesis as protons reenter the cell through the H⁺-ATP synthase complex (1, 12). Although this capability has been demonstrated for haloarchaeal bacteriorhodopsins (18, 19, 26, 27), PR-based photophosphorylation has not been demonstrated previously. Our data demonstrate that illumination of cells expressing a native marine bacterial PR photosystem generates a proton-motive force that does indeed drive cellular ATP synthesis. Under our experimental conditions, 5 min of illumination resulted in a net gain of 2.2×10⁵ ATP molecules per cell. It should be quite possible to utilize the light to biochemical energy conversion enabled by the PR photosystem, for biosynthetic purposes.

The PR photosystem-catalyzed photophosphorylation described here is consistent with a proposed role for PR in marine microbial photoheterotrophy. A few previous studies were unable to detect light-enhanced growth rates or yields in PR-containing isolates grown in seawater or natural seawater incubations (28, 29). In one recent report, light stimulation of growth rate or yield in Pelagibacter ubique, a ubiquitous PR-containing marine planktonic bacterium, could not be detected. These negative results are somewhat difficult to interpret because natural seawater incubations are by necessity chemically undefined, and preferred growth substrates or other limiting nutrients in these experiments were unknown. In contrast, a significant enhancement of both growth rate and yield was recently reported in PR-expressing marine flavobacterium (11) albeit a direct link between PR and the light-induced growth stimulation was not conclusively demonstrated. Our direct observation of an intact PR photosystem gene expression and subsequent photophosphorylation, the recently reported light-enhanced growth rates and yields of PR-containing Flavobacteria (11), and the general ubiquity of PR photosystem genes in diverse microbial taxa of the ocean's photic zone (2-9) all strongly support a significant role for PR-based phototrophy in planktonic marine microorganisms.

In different physiological, ecological, phylogenetic, and genomic contexts, PR activity may benefit cells in a variety of ways, some not directly related to enhanced growth rates or yields. In some bacteria, the H⁺-ATP synthase functions as an ATPase under low respiratory conditions, hydrolyzing ATP and driving proton efflux to maintain the proton-motive force (30). In the light, PR activity could offset this effect and reverse conditions from ATP consumption to ATP production. PR contributions to cellular energy metabolism are likely to be particularly important in starved or substrate-limited cells. Similar to the situation for some Haloarchaea, which use bacteriorhodopsin under oxygen-limiting conditions (18, 19, 26, 27), low respiratory rates may trigger PR expression or activity in marine bacteria, as well. The PR-generated proton-motive force can also be directly coupled to other energy-requiring cellular activities, including flagellar motility or active transport of solutes into or out of the cell (32-34). This phenomenon was recently demonstrated by the coupling of PR activity to flagellar rotation in E. coli (31). Although a sensory function for some PR variants is also a possibility (10, 35), the PR photosystems described here, and the vast majority of others observed to date (1, 3, 4, 7, 8, 11), are not genetically linked to sensory transducers, the hallmark of all known sensory rhodopsins. Most all PRs characterized so far therefore appear to function as light-activated ion pumps.

The marine PR family is remarkably diverse, has a widespread phylogenetic distribution, and is functional in both ether-linked phytanyl and ester-linked fatty acid membrane systems of Archaea and Bacteria (4, 7, 10). A recent survey found that one-third of PR clones examined were colocalized with photopigment biosynthetic operons (7). Operon arrangement and distribution, as well as phylogenetic relationships, suggest that lateral transfer of PR photosystem genes is relatively common among diverse marine microbes (4, 7, 10). The observations reported here demonstrate that acquisition of just a few genes can lead to functional PR photosystem expression and photophosphorylation. The ability of a single lateral transfer event to confer phototrophic capabilities likely explains the ecological and phylogenetic prevalence of these photosystems in nature. In principle, any microorganism capable of synthesizing FPP (a widespread intermediate in isoprenoid biosynthesis) could readily acquire this capability, as we have observed in E. coli.

Apparently, many otherwise chemoorganotrophic microbes in the ocean's photic zone have acquired the ability to use light energy to supplement cellular energy metabolism. The broad array of PR-containing microbes reflects the photosystem's fundamental contribution to cellular bioenergetics, a simplicity and compactness that favors PR photosystem lateral mobility, and a remarkable plasticity that enables photoprotein assembly and function in a diversity of phylogenetic groups and cell membrane types. From a genetic, physiological, and ecological perspective, the transition from heterotrophy to PR-enabled photoheterotrophy seems to represent a relatively small evolutionary step for contemporary microorganisms.

### REFERENCES

1. Beja, O., Aravind, L., Koonin, E. V., Suzuki, M. T., Hadd, A., Nguyen, L. P., Jovanovich, S. B., Gates, C. M., Feldman, R. A., Spudich, J. L., Spudich, E. N. & DeLong, E. F. (2000) Science 289, 1902-1906.
2. Venter, J C, Remington, K, Heidelberg, J F, Halpern, A L, Rusch, D, Eisen, J A, Wu, D, Paulsen, I, Nelson, K E & Nelson, W, et al. (2004) Science 304, 66-74.
3. de la Torre, J. R., Christianson, L. M., Beja, O., Suzuki, M. T., Karl, D. M., Heidelberg, J. & DeLong, E. F. (2003) PNAS 100, 12830-12835.
4. Frigaard, N.-U., Martinez, A., Mincer, T. J. & DeLong, E. F. (2006) Nature 439, 847-850.
5. Man, D., Wang, W., Sabehi, G., Aravind, L., Post, A. F., Massana, R., Spudich, E. N., Spudich, J. L. & Beja, O. (2003) EMBO J. 22, 1725-1731.
6. Man-Aharonovich, D., Sabehi, G., Sineshchekov, O., Spudich, E. N., Spudich, J. L. & Beja, O. (2004) Photochemical and Photobiological Sciences 3, 459-462.
7. McCarren, J & DeLong, E F. (2007) Environ Microbiol 9, 846-58.
8. Sabehi, G., Loy, A., Jung, K.-H., Partha, R., Spudich, J. L., Isaacson, T., Hirschberg, J., Wagner, M. & Beja, O. (2005) PLoS Biology 3, e273.
9. Sabehi, G., Massana, R., Bielawski, J. P., Rosenberg, M., Delong, E. F. & Beja, O. (2003) Environ Microbiol 5, 842-849.
10. Sharma, A. K., Spudich, J. L. & Doolittle, W. F. (2006) Trends Microbiol. 14, 463-9.
11. Gomez-Consarnau, L, Gonzalez, μM, Coll-Llado, M, Gourdon, P, Pascher, T, Neutze, R, Pedros-Alio, C & Pinhassi, J. (2007) Nature 445, 210-213.
12. Beja, O., Spudich, E. N., Spudich, J. L., Leclerc, M. & DeLong, E. F. (2001) Nature 411, 786-789.
13. Wild, J., Hradecna, Z. & Szybalski, W. (2002) Genome Res. 12, 1434-1444.
14. DeLong, E. F., Preston, C. M., Mincer, T., Rich, V., Hallam, S. J., Frigaard, N.-U., Martinez, A., Sullivan, M. B., Edwards, R., Brito, B. R., Chisholm, S. W. & Karl, D. M. (2006) Science 311, 496-503.
15. MacNeil, I. A., Tiong, C. L., Minor, C., August, P. R., Grossman, T. H., Loiacono, K. A., Lynch, B. A., Phillips, T., Narula, S., Sundaramoorthi, R., Tyler, A., Aldredge, T., Long, H., Gilman, M., Holt, D. & Osburne, M. S. (2001) Journal of Molecular Microbiology and Biotechnology 3, 301-308.
16. Armstrong, G. A. (1997) Annual Review of Microbiology 51, 629-659.
17. Hahn, F. M., Hurlburt, A. P. & Poulter, C. D. (1999) Journal of Bacteriology 181, 4499-4504.
18. Danon, A. & Stoeckenius, W. (1974) Proc Natl Acad Sci USA 71, 1234-8.
19. Oesterhelt, D. & Stoeckenius, W. (1973) Proc Natl Acad Sci USA 70, 2853-7.
20. Ito, M., Ohnishi, Y., Itoh, S. & Nishimura, M. (1983) J Bacteriol 153, 310-5.
21. Rondon, M R, August, P R, Bettermann, A D, Brady, S F, Grossman, T H, Liles, M R, Loiacono, K A, Lynch, B A, MacNeil, I A & Minor, C, et al. (2000) Appl Environ Microbiol 66, 2541-2547.
22. Gillespie, D E, Brady, S F, Bettermann, A D, Cianciotto, N P, Liles, M R, Rondon, M R, Clardy, J, Goodman, R M & Handelsman, J. (2002) Appl Environ Microbiol 68, 4301-4306.
23. Fujisaki, S., Takahashi, I., Hara, H., Horiuchi, K., Nishino, T. & Nishimura, Y. (2005) J Biochem (Tokyo) 137, 395-400.
24. Kajiwara, S., Fraser, P. D., Kondo, K. & Misawa, N. (1997) Biochem J 324 (Pt 2), 421-6.
25. Sun, Z., Cunningham, F. X., Jr. & Gantt, E. (1998) Proc Natl Acad Sci USA 95, 11482-8.
26. Brock, T. D. & Petersen, S. (1976) Archives in Microbiology 109, 199-200.
27. Hartmann, R., Sickinger, H. D. & Oesterhelt, D. (1980) Proc Natl Acad Sci USA 77, 3821-5.
28. Giovannoni, S J, Bibbs, L, Cho, J-C, Stapels, M D, Desiderio, R, Vergin, K L, Rappe, M S, Laney, S, Wilhelm, L J & Tripp, H J, et al. (2005) Nature 438, 82-85.
29. Schwalbach, M. S., Brown, M. & Fuhrman, J. (2005) Aquatic Microbial Ecology 39, 235-245.
30. Booth, I. R. (1985) Microbiol Rev 49, 359-78.
31. Walter, J M, Greenfield, D, Bustamante, C & Liphardt, V. (2007) Proc Natl Acad Sci USA 104, 2408-2412.
32. Konings, W. N. (2006) Antonie Van Leeuwenhoek. 90, 325-342.
33. Manson, M. D., Tedesco, P., Berg, H. C., Harold, F. M. & Van der Drift, C. (1977) Proc Natl Acad Sci USA 74, 3060-4.
34. Matsura, S., Shioi, J. & Imae, Y. (1977) FEBS Lett 82, 187-90.
35. Spudich, J. L. (2006) Trends Microbiol. 14, 480-487.
36. Barua, A. B. & Olson, J. A. (1998) Journal of Chromatography, B: Biomedical Sciences and Applications 707, 69-79.
37. Nakano, Y., Yoshida, Y., Yamashita, Y. & Koga, T. (1995). Gene 162, 157-158.

Other aspects of the invention will be clear to the skilled artisan and need not be repeated here. Each reference cited herein is incorporated by reference in its entirety for the relevant teaching contained therein.

The terms and expressions that have been employed are used as terms of description and not of limitation, and there is no intention in the use of such terms and expressions of excluding any equivalents of the features shown and described or portions thereof, it being recognized that various modifications are possible within the scope of the invention.

We claim:

1. A cell comprising a functional heterologous proteorhodopsin photosystem.

2. The cell of claim 1, wherein the cell is a bacterial cell, an archael cell, a yeast cell, a fungal cell or a mammalian cell.

3. The cell of claim 1, wherein the heterologous proteorhodopsin photosystem is introduced into the cell as a set of linked genes on a fosmid or cosmid.

4. The cell of claim 1, wherein the heterologous proteorhodopsin photosystem comprises genes encoding a retinal biosynthesis pathway and proteorhodopsin (PR).

5. The cell of claim 4, wherein the genes encoding the retinal biosynthesis pathway comprise crtE, crtB, crtI, crtY and blh.

6. A method for increasing energy content of a cell comprising
expressing in the cell a functional heterologous proteorhodopsin photosystem, and
exposing the cell to light, whereby the functional heterologous proteorhodopsin photosystem synthesizes adenosine triphosphate (ATP) in the cell, thereby increasing the energy content of the cell.

7. The method of claim 6, wherein the heterologous proteorhodopsin photosystem comprises genes encoding a retinal biosynthesis pathway and proteorhodopsin (PR).

8. The method of claim 7, wherein the genes encoding the retinal biosynthesis pathway comprise crtE, crtB, crtI, crtY and blh.

9. The method of claim 6, wherein the cell is a bacterial cell, an archael cell, a yeast cell, a fungal cell or a mammalian cell.

10. The method of claim 9, wherein the cell is a bacterial cell.

11. The method of claim 10, wherein the bacterial cell is in a stationary phase.

12. A method for increasing production of a metabolite in a cell comprising
expressing in the cell a functional heterologous proteorhodopsin photosystem,
culturing the cell to produce the metabolite, and
exposing the cell to light, whereby the functional heterologous proteorhodopsin photosystem synthesizes adenosine triphosphate (ATP) in the cell, thereby increasing the energy of the cell available for production of the metabolite, wherein the production of the metabolite is increased.

13. The method of claim 12, wherein the heterologous proteorhodopsin photosystem comprises genes encoding a retinal biosynthesis pathway and proteorhodopsin (PR).

14. The method of claim 13, wherein the genes encoding the retinal biosynthesis pathway comprise crtE, crtB, crtI, crtY and blh.

15. The method of claim 12, wherein the cell is a bacterial cell, an archael cell, a yeast cell, a fungal cell or a mammalian cell.

16. The method of claim 15, wherein the cell is a bacterial cell.

17. The method of claim 16, wherein the bacterial cell is in a stationary phase.

18. A method to redirect carbon flow in a cell comprising
expressing in the cell a functional heterologous proteorhodopsin photosystem, and
exposing the cell to light, whereby the functional heterologous proteorhodopsin photosystem synthesizes adenosine triphosphate (ATP) in the cell, thereby redirecting carbon compounds from production of ATP by the cell to production of other compounds by the cell.

19. The method of claim 18, wherein the other compounds are metabolites.

20. The method of claim 18, wherein the heterologous proteorhodopsin photosystem comprises genes encoding a retinal biosynthesis pathway and proteorhodopsin (PR).

21. The method of claim 20, wherein the genes encoding the retinal biosynthesis pathway comprise crtE, crtB, crtI, crtY and blh.

22. The method of claim 18, wherein the cell is a bacterial cell, an archael cell, a yeast cell, a fungal cell or a mammalian cell.

23. The method of claim 22, wherein the cell is a bacterial cell.

24. The method of claim 23, wherein the bacterial cell is in a stationary phase.

25. A method to increase a production of a carbon-based compound in a cell comprising
expressing in the cell a functional heterologous proteorhodopsin photosystem, and
exposing the cell to light, whereby the functional heterologous proteorhodopsin photosystem synthesizes adenosine triphosphate (ATP) in the cell, thereby reducing the amount of carbon compounds needed for production of ATP by the cell, thereby making the carbon compounds available for increased production of the carbon-based compound by the cell.

26. The method of claim 25, wherein the heterologous proteorhodopsin photosystem comprises genes encoding a retinal biosynthesis pathway and proteorhodopsin (PR).

27. The method of claim 26, wherein the genes encoding the retinal biosynthesis pathway comprise crtE, crtB, crtI, crtY and blh.

28. The method of claim 25, wherein the cell is a bacterial cell, an archael cell, a yeast cell, a fungal cell or a mammalian cell.

29. The method of claim 28, wherein the cell is a bacterial cell.

30. The method of claim 29, wherein the bacterial cell is in a stationary phase.
